(19) United States
(12) Patent Application Publication
(10) Pub. No.: US 2022/0296516 A1
(43) Pub. Date: Sep. 22, 2022

(54) COMPOSITIONS AND METHODS FOR BIOLOGICAL DELIVERY VEHICLES

(71) Applicant: DNAlite Therapeutics, Inc., San Francisco, CA (US)

(72) Inventors: Mubhij Ahmad, Walnut Creek, CA (US); Timothy Day, Oakland, CA (US); Ismail Hafez, Concord, CA (US); John Merritt, San Mateo, CA (US)

(21) Appl. No.: 17/619,036

(22) PCT Filed: Jun. 12, 2020
(86) PCT No.: PCT/US2020/037579

§ 371 Date: Dec. 14, 2021

Related U.S. Application Data
(60) Provisional application No. 62/861,852, filed on Jun. 14, 2019.
(60) Provisional application No. 62/948,095, filed on Dec. 13, 2019.

(51) Int. Cl. A61K 9/127; A61K 9/51; A61K 47/24; A61K 47/69

(52) U.S. Cl. CPC A61K 9/1272; A61K 9/5123; A61K 9/5184; A61K 47/24; A61K 47/6909

(57) ABSTRACT

Provided are delivery vehicles, and methods of making and using same for reaching epithelial cells, such as cells within mucus-containing environments, and delivery vehicles with improved stability in harsh environments, including in the gastrointestinal tract.

### CROSS-REFERENCE

This application claims priority to U.S. Provisional Patent Application No. 62/861,852, filed on Jun. 14, 2019, and U.S. Provisional Patent Application No. 62/948,095, filed on Dec. 13, 2019, each of which is entirely incorporated herein by reference for all purposes.

### STATEMENT AS TO FEDERALLY SPONSORED RESEARCH

This invention was made with the support of the United States government under Contract number 1846078 by the National Science Foundation.

### BACKGROUND

Despite advances in gene therapy over the last 50 years, there remain many diseases that are recalcitrant to conventional methods, particularly in cases where a target location for gene therapy may provide challenges for delivery, such as in the gastrointestinal tract. The present disclosure addresses this need and provides a number of advantages as well.

### SUMMARY

Provided herein is a delivery vehicle comprising (i) a cargo and a (ii) lipid nanoparticle, wherein the lipid nanoparticle comprises at least one saturated lipid, and a bile salt, and wherein the at least one saturated lipid is a saturated cationic lipid, or the lipid nanoparticle further comprises at least one cationic lipid. In some cases, the lipid nanoparticle further comprises at least one of an unsaturated cationic lipid or an unsaturated non-cationic lipid, and optionally wherein the concentration of the at least one unsaturated cationic lipid or unsaturated non-cationic lipid in the lipid nanoparticle is less than 50 mole % of the total lipid concentration of the lipid nanoparticle. In some cases, the saturated cationic lipid has a phase transition temperature of at least about 37° C. In some cases, the saturated lipid comprises a saturated non-cationic lipid that has a phase transition temperature of at least about 37° C. In some cases, the lipid nanoparticle further comprises at least one of: a non-cationic lipid, a multivalent cationic lipid, a permanently charged cationic lipid, or any combinations thereof. In some cases, the multivalent cationic lipid comprises at least one of: MVL5, TMVLBG2, TMVLG3, TMVLBG1, GL67, or any combinations thereof. In some cases, the multivalent cationic lipid comprises MVL5. In an aspect, the multivalent cationic lipid is about 25 mole % or less of the total lipid concentration. In some cases, the permanently charged cationic lipid comprises 1,2-dioleoyl-3-trimethylammonium-propane (DOTAP), 3ß-[N—(N′,N′-dimethylaminoethane)-carbamoyl]cholesterol hydrochloride (DC-Cholesterol-HCl), or any combinations thereof. In some cases, the saturated cationic lipid comprises at least one of: 1,2-stearoyl-3-trimethylammonium-propane, 1,2-dipalmitoyl-3 trimethylammonium-propane, 1,2-Distearoyl-3-Dimethylammonium-Propane, Dimethyldioctadecylammonium, 1,2-dialkyl-sn-glycero-3-ethylphosphocholine, 1,2-dialkyl-3-dimethylammonium-propane, 1,2-dialkyl-3-trimethylammonium-propane, 1,2-di-O-alkyl-3-trimethylammonium propane, 1,2-dialkyloxy-3-dimethylaminopropane, N,N-dialkyl-N,N-dimethylammonium, N-(4-carboxybenzyl)-N,N-dimethyl-2,3-bis(alkyloxy)propan-1-aminium, 1,2-dialkyl-sn-glycero-3-[(N-(5-amino-1-carboxypentyl)iminodiacetic acid)succinyl], N1-[2-((1S)-1-[(3-aminopropyl)amino]-4-[di(3-amino-propyl)amino]butylcarboxamido)ethyl]-3,4-di[alkyl]-benzamide, 1,2-stearoyl-3-trimethylammonium-propane (DSTAP), 1,2-dipalmitoyl-3-trimethylammonium-propane (DPTAP), 1,2-Distearoyl-3-Dimethylammonium-Propane (DSDAP), or any combinations thereof. In some cases, the saturated non-cationic lipid comprises at least one of: 1,2-Dialkyl-sn-glycero-3-phosphocholine, 1,2-dialkyl-sn-glycero-3-phosphoethanolamine, 1,2-Diaklyl-sn-glycero-3-phosphorylglycerol, 1,2-dialkyl-sn-glycero-3-Phosphatidylserine, 1,2-dialkyl-sn-glycero-3-Phosphate, Monoglycerol alkylate, Glyceryl hydroxyalkylate, Sorbitan monoalkylate, 1,2-dialkyl-sn-glycero-3-phosphoethanolamine-N-methyl, 1,2-dialkyl-sn-glycero-3-phosphomethanol, 1,2-dialkyl-sn-glycero-3-phosphoethanol, 1,2-dialkyl-sn-glycero-3-phosphoethanolamine-N,N-dimethyl, 1,2-dialkyl-sn-glycero-3-phosphopropanol, 1,2-dialkyl-sn-glycero-3-phosphobutanol, or any combinations thereof. In some cases, the unsaturated cationic lipid comprises at least one of: Dimethyldioctadecylammonium, 1,2-dialkyl-sn-glycero-3-ethylphosphocholine, 1,2-dialkyl-3-dimethylammonium-propane, 1,2-dialkyl-3-trimethylammonium-propane, 1,2-di-O-alkyl-3-trimethylammonium propane, 1,2-dialkyloxy-3-dimethylaminopropane, N,N-dialkyl-N,N-dimethylammonium, N-(4-carboxybenzyl)-N,N-dimethyl-2,3-bis(alkyloxy)propan-1-aminium, 1,2-dialkyl-sn-glycero-3-[(N-(5-amino-1-carboxypentyl)iminodiacetic acid)succinyl], N1-[2-((1S)-1-[(3-aminopropyl)amino]-4-[di(3-amino-propyl)amino]butylcarboxamido)ethyl]-3,4-di[alkyl]-benzamide, 1,2-Dialkyloxy-N,N-dimethylaminopropane, 4-(2,2-diocta-9,12-dienyl-[1,3]dioxolan-4-ylmethyl)-dimethylamine, O-alkyl ethylphosphocholines, MC3, MC2, MC4, 3ß-[N—(N′,N′-dimethylaminoethane)-carbamoyl]cholesterol, N4-Cholesteryl-Spermine, 7-(4-(dimethylamino)butyl)-7-hydroxytridecane-1,13-diyl dioleate (CL1H6), or any combinations thereof. In some cases, the unsaturated cationic lipid comprises at least MC2 or CL1H6. In some cases, the unsaturated non-cationic lipid comprises at least one of: 1,2-Dialkyl-sn-glycero-3-phosphocholine, 1,2-dialkyl-sn-glycero-3-phosphoethanolamine, 1,2-Diaklyl-sn-glycero-3-phosphorylglycerol, 1,2-dialkyl-sn-glycero-3-Phosphatidylserine, 1,2-dialkyl-sn-glycero-3-Phosphate, Monoglycerol alkylate, Glyceryl hydroxyalkylate, Sorbitan monoalkylate, 1,2-dialkyl-sn-glycero-3-phosphoethanolamine-N-methyl, 1,2-dialkyl-sn-glycero-3-phosphomethanol, 1,2-dialkyl-sn-glycero-3-phosphoethanol, 1,2-dialkyl-sn-glycero-3-phosphoethanolamine-N,N-dimethyl, 1,2-dialkyl-sn-glycero-3-phosphopropanol, 1,2-dialkyl-sn-glycero-3-phosphobutanol, or any combinations thereof. In some cases, the at least one saturated lipid or cationic lipid is a multivalent cationic lipid.

In an embodiment, the delivery vehicle further comprises a non-cationic lipid. In some cases, the multivalent cationic lipid, the non-cationic lipid, or the multivalent cationic lipid and the non-cationic lipid have a phase transition temperature of at least about 37° C. In some cases, the multivalent cationic lipid comprises at least one of: MVL5, TMVLBG2, TMVLG3, TMVLBG1, and GL67, or any combinations thereof. In some cases, the non-cationic lipid comprises a saturated non-cationic lipid. In some cases, the saturated non-cationic lipid comprises at least one of: 1,2-Dialkyl-sn-glycero-3-phosphocholine, 1,2-dialkyl-sn-glycero-3-phosphoethanolamine, 1,2-Diaklyl-sn-glycero-3-phosphorylglycerol, 1,2-dialkyl-sn-glycero-3-Phosphatidylserine, 1,2-dialkyl-sn-glycero-3-Phosphate, Monoglycerol alkylate, Glyceryl hydroxyalkylate, Sorbitan monoalkylate, 1,2-dialkyl-sn-glycero-3-phosphoethanolamine-N-methyl, 1,2-dialkyl-sn-glycero-3-phosphomethanol, 1,2-dialkyl-sn-glycero-3-phosphoethanol, 1,2-dialkyl-sn-glycero-3-phosphoethanolamine-N,N-dimethyl, 1,2-dialkyl-sn-glycero-3-phosphopropanol, 1,2-dialkyl-sn-glycero-3-phosphobutanol, or any combinations thereof. In some cases, the delivery vehicle is stable in a high bile salt environment, compared to an otherwise identical delivery vehicle that does not comprise the bile salt. In some cases, the high bile salt environment comprises a gastrointestinal environment. In some cases, the delivery vehicle demonstrates an increased stability in a solution containing at least about 5 g/L of the bile salt, compared to an otherwise identical delivery vehicle that (i) does not comprise the bile salt, wherein the stability is measured by relative fluorescence intensity of a fluorescent lipid incorporated into the lipid nanoparticle, in a Forster resonance energy transfer (FRET) assay. In some cases, the delivery vehicle demonstrates an increased stability in a solution containing at least about 5 g/L of a mixture of about 50% cholic acid and about 50% deoxycholate, compared to an otherwise identical delivery vehicle that (i) does not comprise the bile salt, wherein the stability is measured by relative fluorescence intensity of a fluorescent lipid incorporated into the lipid nanoparticle, in a Forster resonance energy transfer (FRET) assay.

In an embodiment, a delivery vehicles comprises at least one of: N,N-dioleyl-N,N-dimethylammonium chloride (DODAC); N-(2,3dioleyloxy)propyl)-N,N,Ntrimethylammonium chloride (DOTMA); N,NdistearylN,N-dimethylammonium bromide (DDAB); N-(2,3dioleoyloxy)propyl)-N,N,N-trimethylamntonium chloride (DODAP); N-(1,2-dimyristyloxyprop-3-yl)-N,N-dimethyl-N-hydmxyethyl ammonium bromide (DMRIE); 1,2dioleoyl-sn-3-phosphoethanolamine (DOPE); N-(1-(2,3dioleyloxy)propyl)N-(2-(sperminecarboxamido)ethyl)-N,N-dimethylammonium trifluoroacetate (DOSPA); diocmdecylamidoglycyl carboxyspermine (DOGS); 1,2-dioleoyl-3-dimethylammonium-propane (DODAP); DMDMA; 1,2-DiLinoleyloxy-N,N-dimethylaminopropane (DLinDMA); 4-(2,2-diocta-9,12-dienyl-[1,3]dioxolan-4-ylmethyl)-dimethylamine; DLin-K-C2-DMA; DLin-M-C3-DMA; 2-{4-[(3β)-cholest-5-en-3-yloxy]butoxy}-N,N-dimethyl-3-[(9Z,12Z)-octadeca-9,12-dienlyloxyl]propan-1-amine) (CLinDMA), MC4, O-alkyl ethylphosphocholines, Didodecyldimethylammonium bromide (DDAB), N-(4-carboxybenzyl)-N,N-dimethyl-2,3-bis(oleoyloxy)propan-1-aminium (DOBAQ), or any combinations thereof.

In an embodiment, a delivery vehicle comprises at least one of: a diacylphosphatidylcholine, a diacylphosphatidyletbanolamine, a ceramide, a sphingomyelin, a cephalin, a cerebroside, a diacylglycerol, or any combinations thereof.

In an embodiment, a delivery vehicle comprises at least one of: a phosphatidylglycerol, a cardiolipin, a diacylphosphatidylserine, a diacylphosphatidic acid, a N-dodecanoyl phosphatidylethanolamine, a N-succinyl phosphatidylethanolamine, a N-glutarylphosphatidylethanolamine, a lysylphosphatidylglycerol, a palmitoyloleyolphosphatidylglyeerol (POPG), or any combinations thereof.

In an embodiment, a delivery vehicle comprises at least one of: distearoylphosphatidylcholine (DSPC), phosphatidylcholine 1,2-dimyristoyl-sn-glycero-3-phosphocholine (DMPC), 1,2-Distearoyl-sn-glycero-3-phospho-L-serine (DSPS), dioleoylphosphatidylcholine (DOPC), dipalmitoylphosphatidylcholine (OPEC), dioleoylphospbatidylglycerol (DOPG), dipahnitoylphosphatidylglycerol (DPPG), dioleoyl-phosphatidylethanolamine (DOPE), palmitoyloleoylphosphatidylcholine (POPC), palmitoylolmyl-phosphatidylethanolamine (POPE) and dioleoyl-phosphatidylethanolamine 4-(4-maleimidomethyl)cyelohexane-1-carboxylate (DOPE-teal), dipahnitoyl phosphatidyl ethanolamine (DPPE), dimyristoylphosphoetbanolamine (DMPE), distearoylphosphatidylethanolamine (DSPE), 16-O-monomethyl PE, 16-O-dimethyl PE, 18-1-trans PE, 1-stearoyl-2-oleoyl-phosphatidyethanolamine (SOPS), 1,2-dielaidoyl-sn-glycero-3-phophoethanolamine (transDOPE), or any combinations thereof.

In an embodiment, a delivery vehicle comprises at least DSPC or DMPC.

In an embodiment, a delivery vehicle further comprises a conjugated lipid, wherein the conjugated lipid comprises a lipid conjugated to a stabilizing component. In some cases, the stabilizing component comprises a hydrophilic polymer. In some cases, the hydrophilic polymer comprises a polyethylene glycol, a poly(2-alkyl-2-oxazoline), a polyvinyl alcohol, or any combinations thereof. In some cases, the hydrophilic polymer comprises a molecule weight from about 50 kDa to about 500 kDa. In some cases, the hydrophilic polymer comprises the polyethyleneglycol (PEG), and wherein the conjugated lipid comprises a pegylated lipid. In some cases, the pegylated lipid comprises DSPE-PEG, DSG-PEG, DMG-PEG, or DPPE-PEG. In some cases, the pegylated lipid comprises DSPE-PEG or DMG-PEG. In some cases, the concentration of the conjugated lipid is less than 25 mole %. In some cases, the concentration of the conjugated lipid is less than 5 mole %. In some cases, the concentration of the conjugated lipid is from about 0.5 mole % to about 20 mole %. In some cases, the delivery vehicle comprises the non-cationic lipid, and the concentration of the non-cationic lipid is from about 5 mole % to about 75 mole %. In some cases, the lipid nanoparticle comprises a positive or near neutral net charge.

In an aspect, a delivery vehicle, further comprises cholesterol.

Provided herein is a delivery vehicle comprising a cargo and a nanoparticle, wherein the nanoparticle comprises a first locus that is positively charged at a pH between about 5.5 and 8.0, and a second locus that is negatively charged at a pH between about 5.5 and 8.0, wherein the first and second loci are separated such that the positive and negative charges are not interspersed, and wherein the nanoparticle is capable of crossing a mucus barrier and reaching an epithelial cell. In an aspect, reaching an epithelial cell comprises the delivery vehicle coming within a 20 micron proximity of the cell surface, associating with the epithelial cell surface or internalization by the epithelial cell. In an aspect, the nanoparticle comprises a lipid, a polymer or a combination thereof. In an aspect, the first locus is comprised in a first phase and the second locus is comprised in a second phase and wherein the first phase and the second phase are physically separated from one another. In an aspect, the first phase is liquid. In an aspect, the second phase is a gel. In an aspect, the first phase is a gel. In an aspect, the second phase is liquid. In some cases, a delivery vehicle further comprises a stability component. In some cases, the stability component is a polyethylene glycol (PEG). In some cases, the first locus comprises an unsaturated lipid or a short-tail lipid. In some cases, the unsaturated lipid comprises a cationic lipid or an ionizable cationic lipid. In some cases, the cationic lipid comprises a multivalent cationic lipid or a monovalent cationic lipid. In some cases, the cationic lipid is selected from the group consisting of N1-[2-((1S)-1-[(3-aminopropyl)amino]-4-[di(3-amino-propyl)amino]butylcarboxamido)ethyl]-3,4-di[oleyloxy]-benzamide (MVL5), 1,2-Dioleoyl-3-trimethylammonium propane (DOTAP), N4-Cholesteryl-Spermine HCl (GL67), a salt of any of these, and any combination thereof. In an aspect, one or more lipids in the first phase is PEGylated. In an aspect, the first locus further comprises at least one of: 1,2-Dioleyloxy-3-(dimethylamino)propane (DODMA), 6Z,9Z,28Z,31Z)-heptatriaconta-6,9,28,31-tetraen-19-yl 3-(dimethylamino)propanoate (MC2), or any combination thereof. In an aspect, the second locus comprises at least one of: 1,2-Distearoyl-sn-glycero-3-phospho-L-serine (DSPS), 1,2-Dipalmitoyl-sn-glycero-3-phospho-L-serine (DPPS), Depot medroxyprogesterone acetate (DMPA), Diphenylphosphoryl azide (DPPA), 1,2-Distearoyl-sn-glycero-3-phosphatidic acid sodium (DSPA), 1,2-DipalmitoylphosphatidylglycerolDipalmitoylphosphatidylglycerol (DPPG) or 2,4-Diacetylphloroglucinol (DAPG). In an aspect, the second locus further comprises at least one of 2-distearoyl-sn-glycero-3-phosphocholine (DSPC), 1,2-Bis(dimethylphosphino)ethane (DMPE), 1,2-Bis(diphenylphosphino)ethane (DPPE), 1,2-Distearoylphosphatidylethanolamine (DSPE), Dipalmitoylphosphatidylcholine (DPPC), 1,2-Diarachidoyl-sn-glycero-3-phosphocholine 20:0 PC (DAPC), or 1,2-Diradyl-3-phosphatidylethanolamine 20:0 PE (DAPE). In an aspect, the second locus comprises deoxycholate, and at least one of 2-distearoyl-sn-glycero-3-phosphocholine (DSPC), 1,2-Bis(dimethylphosphino)ethane (DMPE), 1,2-Bis(diphenylphosphino)ethane (DPPE), 1,2-Distearoylphosphatidylethanolamine (DSPE), Dipalmitoylphosphatidylcholine (DPPC), 1,2-Diarachidoyl-sn-glycero-3-phosphocholine 20:0 PC (DAPC), or 1,2-Diradyl-3-phosphatidylethanolamine 20:0 PE (DAPE). In an aspect, the first phase has a transition temperature below 37° C., and the second phase has a transition temperature above 37° C. In an aspect, the first phase has a transition temperature above 37° C. and the second phase has a transition temperature below 37° C. In an aspect, the phase that has the transition temperature below 37° C. comprises DODMA, MVL5, MC2, a cationic lipid or an ionizable cationic lipid. In an aspect, the phase that has the transition temperature above 37° C. comprises DSPC. In some cases, the ratio of the cationic charge in the first locus to the anionic charge in the second locus at pH 7.4 is between about 0.25 and about 3.0. In some cases, the ratio is between about 0.75 and about 1.25. In some cases, first phase comprises MVL5 and an ionizable cationic lipid. In some cases, the ionizable cationic lipid is selected from the group consisting of DODMA, MC2, MC3, and KC2. In some cases, the ionizable cationic lipid is DODMA or MC2 and the mole % ratio of MVL5:ionizable cationic lipid in the delivery vehicle is about 6.25%:18.75%, 12.5%:12.5%, or 18.75%:6.25%. In some cases, the ratio of MVL5:ionizable cationic lipid in the delivery vehicle is about 12.5%:12.5%. In some cases, the second phase comprises deoxycholate.

In an aspect, a delivery vehicle further comprises 2-dimyristoyl-rac-glycero-3-methoxypolyethylene glycol (DMG-PEG), or a salt thereof. In some cases, a delivery vehicle further comprises DMPE-PEG, or a salt thereof. In some cases, the first locus comprises a cationic lipid and the second locus comprises an anionic compound. In some cases, the cationic lipid is MVL5. In some cases, the anionic compound comprises a bile salt. In some cases, the bile salt is selected from the group consisting of cholic acid, cholate, deoxycholic acid, deoxycholate, hyodeoxycholic acid, hyodeoxycholate, glycocholic acid, glycocholate, taurocholic acid, taurocholate, chenodeoxycholic acid, chenodeoxycholate, isolithocholic acid, isolithocolate, lithocholic acid, and lithocolate. In some cases, the bile salt is selected from the group consisting of lithocolate, deoxycholate and isolithocolate. In some cases, the bile salt is deoxycholate. In some cases, the bile salt is isolithocolate. In some cases, the bile salt is at a concentration of from about 10 mole % to about 80 mole %. In some cases, the cargo is at least partially encompassed by the lipid nanoparticle. In some cases, the cargo comprises a therapeutic agent. In some cases, the cargo comprises a nucleic acid, a protein, an antibody, a peptide, a small molecule, a biologic or any combination thereof. In some cases, the cargo is a nucleic acid and the nucleic acid comprises DNA, modified DNA, RNA, modified RNA, miRNA, siRNA, antisense RNA, or any combinations thereof. In some cases, a delivery vehicle further comprises a component for cell internalization. In an aspect, the component is a peptide, a carbohydrate or ligand.

In an aspect, a delivery vehicle further comprises a cell penetrating peptide, a ligand, a mucus penetrating polymer, mucus penetrating peptide, non-mucus adhesive cell penetrating peptide, or any combinations thereof.

Provided herein is a pharmaceutical composition comprising a delivery vehicle.

Provided herein is a method of delivering cargo to the gastrointestinal tract comprising administering a delivery vehicle or pharmaceutical composition, wherein the delivery vehicle reaches the gastrointestinal tract and wherein the delivery vehicle protects the cargo from bile salts present in the gastrointestinal tract. In some cases, a delivery vehicle facilitates crossing a mucus barrier. In some cases, a delivery vehicle is capable of reaching an epithelial cell within the gastrointestinal tract. In some cases, reaching an epithelial cell comprises the delivery vehicle coming within a 20 micron proximity of the cell surface. In some cases, a delivery vehicle contacts the surface of an epithelial cell. In an aspect, subsequent to the delivery vehicle contacting the epithelial cell, the cargo is internalized by the epithelial cell. In some cases, the delivery vehicle or pharmaceutical composition is administered orally or parenterally to a subject in need thereof. In some cases, the cargo comprises a nucleic acid, a protein, an antibody, a peptide, a small molecule or a biologic. In some cases, the nucleic acid encodes a therapeutic agent and wherein the epithelial cells express the therapeutic agent subsequent to internalization of the cargo. In some cases, the therapeutic agent is secreted by the epithelial cell.

### INCORPORATION BY REFERENCE

All publications, patents, and patent applications herein are incorporated by reference in their entirety to the same extent as if each individual publication, patent, or patent application was specifically and individually indicated to be incorporated by reference. In the event of a conflict between a term herein and a term in an incorporated reference, the term herein controls.

### BRIEF DESCRIPTION OF THE DRAWINGS

The novel features of the disclosure are set forth with particularity in the appended claims. A better understanding of the features and advantages of the disclosure will be obtained by reference to the following detailed description that sets forth illustrative embodiments, in which the principles of the disclosure can be utilized, and the accompanying drawings of which:

FIG. 1 shows the results of an exemplary assay to measure transfection efficiency of exemplary delivery vehicles of this disclosure, carrying DNA as cargo, in HEK cells.

FIG. 2 shows results of an exemplary assay for measuring stability of exemplary delivery vehicles of this disclosure.

FIG. 3 shows results of an exemplary assay for measuring stability of exemplary delivery vehicles of this disclosure.

FIG. 4 shows results of an exemplary assay for measuring stability of exemplary delivery vehicles of this disclosure.

FIG. 5 shows an agarose gel electrophoresis with an exemplary delivery vehicle of this disclosure (Formulation No. 5 in Table 1). The lanes from left are as follows: lane one shows the ladder; lane 2 shows untreated delivery vehicle; lane three shows delivery vehicle treated with 7% Triton-X 100; lane four shows delivery vehicle treated with 7% Triton-X plus heat (70° C. for 30 mins).

FIG. 6 shows a mouse colon section of a mouse dosed with 30 micrograms of DNA encapsulated in a delivery vehicle that was DiI and DiO labelled. Observed is the distribution of 1% PEG containing vehicle (particle 5 of Table 3) labelled with DiI and DiO as shown by fluorescence imaging from DiI overlaid onto brightfield. See Example 5, Table 3 for descriptions of particle 5 and other referenced particles in the Figures.

FIG. 7 shows a mouse colon section of a mouse dosed with 30 micrograms of DNA encapsulated in a delivery vehicle that was DiI and DiO labelled. Observed is the distribution of 2% PEG containing vehicle (particle 6 of Table 3) labelled with DiI and DiO as shown by fluorescence imaging from DiI overlaid onto brightfield.

FIG. 8 shows a mouse colon section of a mouse dosed with 30 micrograms of DNA encapsulated in a delivery vehicle (particle 7 of Table 3) that was DiI and DiO labelled. Observed is the distribution of 3% PEG containing vehicle labelled with DiI and DiO as shown by fluorescence imaging from DiI overlaid onto brightfield.

FIG. 9 shows a mouse colon section of a mouse dosed with 30 micrograms of DNA encapsulated in a delivery vehicle that was DiI and DiO labelled. Observed is the distribution of 5% PEG containing vehicle (particle 8 of Table 3) labelled with DiI and DiO as shown by fluorescence imaging from DiI overlaid onto brightfield.

FIG. 10 shows a mouse colon section of a mouse dosed with 30 micrograms of DNA encapsulated in a delivery vehicle that was DiI and DiO labelled. Observed is the distribution of 10% PEG containing vehicle (particle 9 of Table 3) labelled with DiI and DiO as shown by fluorescence imaging from DiI overlaid onto brightfield.

FIG. 11A and FIG. 11B show distribution of delivery vehicles in representative colon sections from mice administered particles at a ratio of 0%/25% MVL5/DODMA percent mols (particle 1 of Table 3).

FIG. 12A and FIG. 12B show distribution of delivery vehicles in representative colon sections from mice administered particles at a ratio of 6.25%/18.75% (MVL5/DODMA) percent mols (particle 2 of Table 3).

FIG. 13A and FIG. 13B show distribution of delivery vehicles in representative colon sections from mice administered particles at a ratio of 12.5%/12.5% (MVL5/DODMA) percent mols (particle 3 of Table 3).

FIG. 14A and FIG. 14B show distribution of delivery vehicles in representative colon sections from mice administered particles at a ratio of 18.75%/6.25% (MVL5/DODMA) % mols (particle 4).

FIG. 15A and FIG. 15B show distribution of delivery vehicles in representative colon sections from mice administered particles at a ratio of 25%/0% MVL5/DODMA % mols (particle 10 of Table 3).

FIG. 16A and FIG. 16B show swiss roll images of colons of a section of a first mouse and FIG. 16C and FIG. 16D show swiss roll images of colons of a section of a second mouse, each mouse was administered MVL5/DODMA/DOPC/Deoxycholate/DMG-PEG (particle 11 of Table 3) with DiI and DiO using the BioTek Cytation software. FIG. 16A and FIG. 16C show the DiI channel and FIG. 16B and FIG. 16D show the DiI channel overlaid onto brightfield.

FIG. 17A and FIG. 17B show swiss roll images of colons of a section of a first mouse and second mouse (FIG. 17C and FIG. 17D) administered MVL5/DODMA/GMO/Deoxycholate/DMG-PEG (particle 12 of Table 3) with DiI and DiO using the BioTek Cytation software. FIG. 17A and FIG. 17C show the DiI channel and FIG. 17B and FIG. 17D show the DiI channel overlaid onto brightfield.

FIG. 18A and FIG. 18B show swiss roll images of colons of a section of a first mouse and second mouse (FIG. 18C and FIG. 18D) administered MVL5/DODMA/DSPC/Deoxycholate/DMG-PEG (particle 5 of Table 3) with DiI and DiO using the BioTek Cytation software. FIG. 18A and FIG. 18C show the DiI channel and FIG. 18B and FIG. 18D show the DiI channel overlaid onto brightfield.

FIG. 19A and FIG. 19B show swiss roll images of colons of a section of a first mouse and FIG. 19C and FIG. 19D show swiss roll images of colons of a section of a second mouse, each administered PBS with DiI and DiO using the BioTek Cytation software. FIG. 19A and FIG. 19C show the DiI channel and FIG. 19B and FIG. 19D show the DiI channel overlaid onto brightfield.

FIG. 20 shows a bar graph comparing the stability of different bile salts incorporating lipid structures in 10 g/L of bile salts (cholate: deoxycholate mixture) by measuring perturbations in the lipid structures using FRET between DiI and DiO. FRET values are normalized to no treatment.

### DETAILED DESCRIPTION OF THE DISCLOSURE

The following description and examples illustrate embodiments of the disclosure in detail. It is to be understood that this disclosure is not limited to the particular embodiments described herein and as such can vary. Those of skill in the art will recognize that there are numerous variations and modifications of the disclosure, which are encompassed within its scope.

### Overview

Delivery of agents, such as therapeutic agents to epithelial tissues and cells, such as in the gastrointestinal (GI) tract, vagina and lung, present certain challenges. In these tissues, epithelial cells are covered with a mucosal layer and thus therapeutic agents must penetrate and move through the mucus to reach the epithelial cells. Additionally, therapeutic agents, once within or through the layer of mucus, must come into proximity to the intended target cells and in some cases, interact with the cell membrane and/or enter the cells. Accordingly, delivery of an agent (also referred to herein as “cargo”) is improved with a delivery vehicle that not only penetrate and cross through the mucus layer but also come within reach of the intended epithelial cell target. Also, with respect the GI tract and other tissues, harsh environments, such as naturally present bile acids of the GI, can present a challenge for the stability of delivery and for successful delivery of cargo to the intended target cells.

Provided herein are compositions (“delivery vehicles”) for and methods of delivering a cargo using delivery vehicles provided herein. In some aspects, delivery vehicles may be further modified to provide stability and/or to reach target epithelial cells in challenging environments. In embodiments, the delivery vehicles provided herein (also referred to herein as “mucosal epithelial reaching” and charge-separated” delivery vehicles) include those with a separation of positive and negative charges into separate loci within the vehicle, such that positively charged and negatively charged molecules are separated from one another, rather than interspersed. The charge-separated delivery vehicles herein provide both penetration through mucus thereby reducing or preventing entrapment of the delivery vehicle in the epithelial mucus as well as the epithelial reaching functionality which brings the delivery vehicle in proximity of the epithelial cells, such as within a distance of 20 microns or less.

Disclosed herein are also delivery vehicles, including lipid based delivery vehicles, comprising lipid structures, such as lipid nanoparticles and a cargo, that have improved stability in high bile salt environments, such as in the gastrointestinal tract. The delivery vehicle, in some embodiments, can provide stability in harsh environments of the GI tract and can be further be suited for mucus environments. As such, the delivery vehicle can be suitable for delivering a cargo (e.g., a nucleic acid) to mucosal epithelial cells such as intestinal epithelial cells, lung epithelial cells, cervical epithelial cells, rectal epithelial cells, endometrial cells and the likes. Further, the delivery vehicle can also be suitable for delivery to organs, such as the skin.

In some cases, the delivery vehicles provided herein can comprise additional mucus-penetrating features that may assist in the penetration and movement of the delivery vehicle through the mucus surrounding the epithelial cells. Such additional features include incorporating a polymer such as Polyethylene glycol (PEG), Polyoxazoline polymer with methyl (PMOZ), Polyoxazoline polymer with ethyl (PEOZ) into the delivery vehicle surface and/or by including a mucus penetrating peptide (MPP) linked to the surface of the delivery vehicle. In other cases, the vehicles contain no PEG coating or a low density PEG coating (or a low density coating of another polymer).

### Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.” The term “about” or “approximately” can mean within an acceptable error range for the particular value as determined by one of ordinary skill in the art, which will depend in part on how the value is measured or determined, e.g., the limitations of the measurement system. For example, “about” can mean within +10% the given value. Where particular values are described in the application and claims, unless otherwise stated the term “about” should be assumed to mean an acceptable error range for the particular value.

The term “about” and its grammatical equivalents in relation to a reference numerical value and its grammatical equivalents as used herein can include a range of values plus or minus 10% from that value. For example, the amount “about 10” includes amounts from 9 to 11. The term “about” in relation to a reference numerical value can also include a range of values plus or minus 10%, 9%, 8%, 7%, 6%, 5%, 4%, 3%, 2%, or 1% from that value.

The term “administering”, and its grammatical equivalents can refer to any method of providing a structure described herein to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a structure disclosed herein can be administered therapeutically. In some instances, a structure can be administered to treat an existing disease or condition. In further various aspects, a structure can be administered prophylactically to prevent a disease or condition.

The term “biodegradable” and its grammatical equivalents can refer to polymers, compositions and formulations, such as those described herein that are intended to degrade during use. The term “biodegradable” is intended to cover materials and processes also termed “bioerodible.”

The term “cancer” and its grammatical equivalents as used herein can refer to a hyperproliferation of cells whose unique trait-loss of normal controls-results in unregulated growth, lack of differentiation, local tissue invasion, and metastasis. With respect to the inventive methods, the cancer can be any cancer, including any of acute lymphocytic cancer, acute myeloid leukemia, alveolar rhabdomyosarcoma, bladder cancer, bone cancer, brain cancer, breast cancer, cancer of the anus, anal canal, rectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, Hodgkin lymphoma, hypopharynx cancer, kidney cancer, larynx cancer, leukemia, liquid tumors, liver cancer, lung cancer, lymphoma, malignant mesothelioma, mastocytoma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, ureter cancer, and/or urinary bladder cancer. As used herein, the term “tumor” refers to an abnormal growth of cells or tissues, e.g., of malignant type or benign type.

The term “cargo” as used herein can refer to one or more molecules or structures encompassed in a delivery vehicle for delivery to or into a cell or tissue. Non-limiting examples of cargo can include a nucleic acid, a dye, a drug, a protein, a liposome, a small chemical molecule, a large biological molecule, and any combinations thereof.

The term “cell” and its grammatical equivalents as used herein can refer to a structural and functional unit of an organism. A cell can be microscopic in size and can consist of a cytoplasm and a nucleus enclosed in a membrane. A cell can refer to an intestinal crypt cell. A crypt cell can refer to the crypts of Lieberkühn which are pit-like structures that surround the base of the villi in the intestine. A cell can be of human or non-human origin. “Conjugate” as used herein can refer to the association, covalently or non-covalently of two or more molecules or structures, including without limitation, the association of a peptide, such as a mucus-penetrating peptide (MPP) with a delivery vehicle, a polymer, a surface modification, or any combinations thereof.

The term “function” and its grammatical equivalents as used herein can refer to the capability of operating, having, or serving an intended purpose. Functional can comprise any percent from baseline to 100% of an intended purpose. For example, functional can comprise or comprise about 5, 10, 15, 20, 25, 30, 35, 40, 45, 50, 55, 60, 65, 70, 75, 80, 85, 90, 95, or up to about 100% of an intended purpose. In some cases, the term functional can mean over or over about 100% of normal function, for example, 125, 150, 175, 200, 250, 300%, 400%, 500%, 600%, 700% or up to about 1000% of an intended purpose.

The term “gastrointestinal disease” as used herein can refer to diseases involving the gastrointestinal tract, including but not limited to esophagus, stomach, small intestine, large intestine and rectum, and the accessory organs of digestion, the liver, gallbladder, and pancreas, and any combinations thereof.

The term “hydrophilic” and its grammatical equivalents as used herein refers to substances or structures that have polar groups that readily interact with water.

The term “hydrophobic” and its grammatical equivalents as used herein refers to substances or structures that have polar groups that do not readily interact with water.

The term “mucus,” and its grammatical equivalents as used herein, can refer to a viscoelastic natural substance containing primarily mucin glycoproteins and other materials, which protects epithelial surface of various organs/tissues, including but not limited to respiratory, nasal, cervicovaginal, gastrointestinal, rectal, visual and auditory systems.

The term “lipid structure” as used herein refers to a lipid composition for delivery to a cell or tissue, such as to deliver a therapeutic product, such as a nucleic acid. The term “lipid structure” and its grammatical equivalents as used herein can refer to a nanoparticle or delivery vehicle. A structure can be a liposomal structure. A lipid structure can also refer to a particle. A lipid structure or particle can be a nanoparticle or delivery vehicle. A lipid particle or lipid structure can be of any shape having a diameter from about 1 nm up to about 1 micron. A nanoparticle or nanostructure can be or can be about 100 to 200 nm. A nanoparticle or nanostructure can also be up to 500 nm. Nanoparticles or nanostructures having a spherical shape can be referred to as “nanospheres”.

The term “structure” and its grammatical equivalents as used herein can refer to a nanoparticle or delivery vehicle. A structure can be a liposomal structure. A structure can also refer to a particle. A structure or particle can be a nanoparticle or delivery vehicle. A particle or structure can be of any shape having a diameter from about 1 nm up to about 1 micron. A nanoparticle or nanostructure can be or can be about 100 to 200 nm. A nanoparticle or nanostructure can also be up to 500 nm. Nanoparticles or nanostructures having a spherical shape can be referred to as “nanospheres”.

The terms “nucleic acid,” “polynucleotide,” and “oligonucleotide” and their grammatical equivalents can be used interchangeably and can refer to a deoxyribonucleotide and/or ribonucleotide polymer, in linear or circular conformation, and in either single- or double-stranded form. For the purposes of the present disclosure, these terms should not to be construed as limiting with respect to length. The terms can also encompass known analogues of natural nucleotides, as well as nucleotides that are modified in the base, sugar and/or phosphate moieties (e.g., phosphorothioate backbones). In general, an analogue of a particular nucleotide can have the same base-pairing specificity, i.e., an analogue of adenine “A” can base-pair with thymine “T”.

The term “pharmaceutically acceptable carrier” and their grammatical equivalents can refer to sterile aqueous or non-aqueous solutions, dispersions, suspensions or emulsions, as well as sterile powders for reconstitution into sterile injectable solutions or dispersions just prior to use. Proper fluidity can be maintained, for example, by the use of coating materials such as lecithin, by the maintenance of the required particle size in the case of dispersions and by the use of surfactants. These solutions, dispersions, suspensions or emulsions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly (orthoesters) and poly (anhydrides).

The term “predisposed” as used herein can be understood to mean an increased probability (e.g., at least 1%, 5%, 10%, 20%, 30%, 40%, 50%, 60%, 70%, 80%, 90%, 100%, 150%, 200%, or more increase in probability) that a subject will suffer from a disease or condition.

The terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker). A subject can be a mammal. A subject can be a human male or a human female. A subject can be of any age. A subject can be an embryo. A subject can be a newborn or up to about 100 years of age. A subject can be in need thereof. A subject can have a disease such as cancer.

The term “sequence” and its grammatical equivalents as used herein can refer to a nucleotide sequence, which can be DNA and/or RNA; can be linear, circular or branched; and can be either single-stranded or double stranded. A sequence can be of any length, for example, between 2 and 1,000,000 or more nucleotides in length (or any integer value there between or there above), e.g., between about 100 and about 10,000 nucleotides or between about 200 and about 500 nucleotides. In some instances, where indicated “sequence” as used herein can refer to an amino acid sequence, such as a sequence of a protein, polypeptide and/or peptide.

The term “stem cell” as used herein, can refer to an undifferentiated cell of a multicellular organism that is capable of giving rise to indefinitely more cells of the same type. A stem cell can also give rise to other kinds of cells by differentiation. Stem cells can be found in crypts. Stem cells can be progenitors of epithelial cells found on intestinal villi surface. Stem cells can be cancerous. A stem cell can be totipotent, unipotent or pluripotent. A stem cell can be an induced stem cell.

The terms “treatment” or “treating” and their grammatical equivalents can refer to the medical management of a subject with the intent to cure, ameliorate, stabilize, or prevent a disease, condition, or disorder. Treatment can include active treatment, that is, treatment directed specifically toward the improvement of a disease, condition, or disorder. Treatment can include causal treatment, that is, treatment directed toward removal of the cause of the associated disease, condition, or disorder. In addition, this treatment can include palliative treatment, that is, treatment designed for the relief of symptoms rather than the curing of the disease, condition, or disorder. Treatment can include preventative treatment, that is, treatment directed to minimizing or partially or completely inhibiting the development of a disease, condition, or disorder. Treatment can include supportive treatment, that is, treatment employed to supplement another specific therapy directed toward the improvement of the disease, condition, or disorder. In some instances, a condition can be pathological. In some instances, a treatment may not completely cure, ameliorate, stabilize or prevent a disease, condition, or disorder.

When used in the context of a chemical group, “hydrogen” means H; “hydroxy” means OH; “halogen” means independently —F, —Cl, —Br or —I;

For the structures provided herein, the following parenthetical subscripts further define the groups as follows: “(Cₙ)” defines the exact number (n) of carbon atoms in the group. For example, “(C₂₋₁₀) alkyl designates those alkyl groups having from 2 to 10 carbon atoms (e.g., 2, 3, 4, 5, 6, 7, 8, 9, or 10, or any range derivable therein (e.g., 3 to 10 carbon atoms).

An “alkyl” group can refer to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic. Furthermore, the alkyl moiety, whether saturated or unsaturated, may comprise branched, straight chain, and/or cyclic portions. Depending on the structure, an alkyl group can be a monoradical or a diradical (i.e., an alkylene group). A “heteroalkyl” group is as described for “alkyl” with at least one of the C atoms thereof substituted with an N, S, or O atom. The “heteroalkyl” group may comprise linear, branched, and/or cyclic portions. In certain embodiments, a “lower alkyl” is an alkyl group with 1-6 carbon atoms (i.e., a C₁-C₆ alkyl group). In specific instances, the “lower alkyl” may be straight chained or branched.

“Aryl” refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. In some embodiments, the term “aryl” can refer to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, naphthalenyl, phenanthrenyl, anthracenyl, fluorenyl, and indenyl. Depending on the structure, an aryl group can be a monoradical or a diradical (i.e., an arylene group).

“Heteroaryl” refers to a radical derived from a 3- to 12-membered aromatic ring radical that comprises two to eleven carbon atoms and at least one heteroatom wherein each heteroatom may be selected from N, O, and S. As used herein, the heteroaryl ring may be selected from monocyclic or bicyclic and fused or bridged ring systems rings wherein at least one of the rings in the ring system is aromatic, i.e., it contains a cyclic, delocalized (4n+2) π-electron system in accordance with the Hückel theory. The heteroatom(s) in the heteroaryl radical may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). An “X-membered heteroaryl” refers to the number of endocylic atoms, i.e., X, in the ring. For example, a 5-membered heteroaryl ring or 5-membered aromatic heterocycle has 5 endocyclic atoms, e.g., triazole, oxazole, thiophene, etc.

In some embodiments, the term “heteroaryl” when used without the “substituted” modifier refers to a monovalent group with an aromatic carbon atom or nitrogen atom as the point of attachment, said carbon atom or nitrogen atom forming part of an aromatic ring structure wherein at least one of the ring atoms is nitrogen, oxygen or sulfur, and wherein the monovalent group consists of no atoms other than carbon, hydrogen, aromatic nitrogen, aromatic oxygen and aromatic sulfur. Non-limiting examples of heteraryl groups include acridinyl, furanyl, imidazoimidazolyl, imidazopyrazolyl, imidazopyridinyl, imidazopyrimidinyl, indolyl, indazolinyl, methylpyridyl, oxazolyl, phenylimidazolyl, pyridyl, pyrrolyl, pyrimidyl, pyrazinyl, quinolyl, quinazolyl, quinoxalinyl, tetrahydroquinolinyl, thienyl, triazinyl, pyrrolopyridinyl, pyrrolopyrimidinyl, pyrrolopyrazinyl, pyrrolotriazinyl, pyrroloimidazolyl, chromenyl (where the point of attachment is one of the aromatic atoms), and chromanyl (where the point of attachment is one of the aromatic atoms). Substituted heteroaryl refers to a monovalent group with an aromatic carbon atom or nitrogen atom as the point of attachment, said carbon atom or nitrogen atom forming part of an aromatic ring structure wherein at least one of the ring atoms is nitrogen, oxygen or sulfur, and wherein the monovalent group further has at least one atom independently selected from the group consisting of non-aromatic nitrogen, non-aromatic oxygen, non-aromatic sulfur F, Cl, Br, I, Si, and P.

The term “substituted” refers to moieties having substituents replacing a hydrogen on one or more carbons or substitutable heteroatoms, e.g., NH, of the structure. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{a}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

### Delivery Vehicles with Charge Separation

In some cases, delivery vehicles provided herein contain positive and negative charges separated into different loci within the particle, where each locus is comprised of a different polymer (conferring the charge to the locus). In some cases, delivery vehicles provided herein contain positively-charged and negatively-charged lipids, where the loci are separated by phase, such as into a liquid phase and a gel phase. In some instances, the delivery vehicle can comprise a positively charged liquid phase and a negatively charged gel phase; or, a positively charged gel phase and a negatively charged liquid phase.

Delivery vehicles provided herein can efficiently deliver cargo, such as nucleic acids, proteins, peptides, and/or small molecules to epithelial cells within mucosal tissues. Delivery vehicles herein are useful to treat diseases and conditions that effect and/or originate in mucosal tissues, such as in the mucosal tissues in the gastrointestinal tract. Non-limiting examples include familial adenomatous polyposis (FAP), attenuated FAP, colorectal cancer, chronic inflammatory bowel disease, chronic inflammatory bowel disease, microvillus inclusion disease and congenital diarrheal diseases. Delivery vehicles herein also are useful to provide therapeutic agents and/or nucleic acids to express therapeutic agents in mucosal tissues and such agents may remain in the targeted epithelial cells and/or be transported to other disease-affected cells and tissues within a subject. In some cases, the delivery vehicle provides a proximity distance to an epithelial cell. In some aspects, such proximity distance is less than about 50, 40, 30, 25, 20, 19, 18, 17, 16, 15, 14, 13, 12, 11, 10, 9, 8, 7, 6, 5, 4, 3, 2, or 1 microns. In some cases, the delivery vehicles herein come in contact with the epithelial cell. In some cases, the delivery vehicle is internalized into the cell and a cargo carried by the delivery vehicle is released within the cell. In some cases, the delivery vehicle contacts the epithelial cell and a cargo from the delivery vehicle is released outside of the cell.

The delivery vehicle provided herein can be a lipid structure. A lipid structure can be utilized for delivery of cargo to a cell or tissue. In some cases, cargo can encompass a therapeutic product, such as a nucleic acid. Lipid structures include, but not are limited to, lipid particles, lipid nanoparticles, liposomes or vesicles, such as vesicles wherein an aqueous volume is encapsulated by amphipathic lipid bilayers (e.g., single; unilamellar or multiple; multilamellar), or wherein the lipids at least partially coat an interior comprising a therapeutic product, or lipid aggregates or micelles, wherein the lipid encapsulated therapeutic product is contained within a relatively disordered lipid mixture.

The delivery vehicles herein (such as lipid nanoparticles, liposomes and micelle-like structures) have at least two loci, and comprise a positive charge and a negative charge that are not interspersed but instead located in separated loci. For example, a negative charge and a positive charge may be present on opposite loci on a lipid structure provided herein at a pH between about 5.5 and 8.0, such as at a pH of about 7.4.

In an aspect, a positive charge and a negative charge are in two separate loci where each locus is a different phase of a lipid structure, for example a liquid or solid (gel) phase. In an aspect, a positive charge may be on a liquid phase and a negative charge may be on a solid phase, for example a gel phase or vice versa. Charge separation can allow for both an attraction and repulsion force. In some cases, a positive lipid can be attracted towards a target cell due to its high negative potential. In another aspect, a repulsive force on a negative face can prevent a positive face from being kinetically trapped in mucus. In some cases, a cationic charge, for instance on a lipid on a delivery vehicle, maybe attracted to mucus, en route to a target cell, and may get kinetically trapped in the mucus thereby trapping the delivery vehicle. The mucus will eventually slough off clearing the delivery vehicle. In another aspect, an anionic delivery vehicle can be repulsed by mucus and may not make its way through the mucus. A zwitterionic particle can act like a neutral particle absent a net force. Zwitterionic particles may follow the flow of water similar to PEGylated systems and may not become trapped in the mucus, but may not reach the epithelial cells.

In particular embodiments, a lipid structure can include one or more of an anionic lipid or cationic lipid, a neutral lipid, a sterol, and a lipid selected to reduce aggregation of lipid particles during formation. Aggregation may result from steric stabilization of lipid structures which may prevent charge-induced aggregation during formation. Lipid structures can include two or more cationic lipids. In an aspect, a cationic lipid may be on a first phase and an anionic lipid on a second phase such that the lipid structure contains two phases with differentially charged lipids. The lipids can be selected to contribute different advantageous properties. For example, cationic lipids that differ in properties such as amine pKₐ, chemical stability, half-life in circulation, half-life in tissue, net accumulation in tissue, or toxicity can be used in a lipid structure. In particular, cationic lipids can be chosen so that the properties of the mixed-lipid lipid structure are more desirable than the properties of a single-lipid structure of individual lipids. Net tissue accumulation and long-term toxicity (if any) from cationic lipids can be modulated in a favorable way by choosing mixtures of cationic lipids instead of selecting a single cationic lipid in a given formulation. Such mixtures can also provide better encapsulation and/or release of a cargo, such as a nucleic acid. A combination of cationic lipids also can affect the systemic stability when compared to single entity in a formulation.

In some cases, a cationic lipid may attain a positive charge through one or more amines present in a polar head group. In some cases, a lipid structure can be a cationic liposome. In some cases, a liposome may be a cationic liposome used to carry negatively charged polynucleic acid, such as DNA. The presence of positively charged amines may facilitate binding with anions such as those found in DNA. A liposome thus formed may be a result of energetic contributions by Van der Waals forces and electrostatic binding to a DNA cargo which may partially contribute to liposome shape. In some cases, a cationic (and neutral) lipid may be used for gene delivery. In other cases, an anionic liposome may be used to deliver other therapeutic agents.

In some embodiments, the delivery vehicles provided herein further comprise a cargo. In some cases, the cargo comprises a therapeutic agent. In some cases, the cargo comprises a nucleic acid, a protein, an antibody, a peptide, a small molecule, a biologic or a combination of any thereof. In some embodiments, the delivery vehicles herein include a component for cell internalization. In some cases, the component is a peptide, a carbohydrate or ligand. In some embodiments, the delivery vehicles provided herein also include a stability component. In some cases, the stability component is a polyethylene glycol (PEG).

In some embodiments of the delivery vehicles, the first locus comprises an unsaturated or short-tail lipid. In some cases, the unsaturated lipid comprises a cationic or ionizable cationic lipid. In some embodiments, the cationic lipid comprises a multivalent cationic lipid or a monovalent cationic lipid.

In some cases, charge separation may result in superior and/or unexpected performance of subject delivery vehicles. For example, utilizing PEG is thought to increase trafficking to target cells, for example intestinal epithelial cells as provided in Maisel K et al., Effect of surface chemistry on nanoparticle interaction with gastrointestinal mucus and distribution in the gastrointestinal tract following oral and rectal administration in the mouse. J Control Release, herein incorporated by reference. In some cases, increasing PEGylation results in decreased distribution within or at the intestinal tissue thereby providing support for utilizing delivery vehicles with reduced PEGylation as compared to conventional vehicles. One mechanism by which reducing PEGylation may improve trafficking and/or distribution to and in proximity to a target cell is by increasing the exposure of positive charge at the surface of a subject vehicle by reducing the shielding properties of PEGylation.

In some cases, a delivery vehicle that comprises charge separation as provided herein can have improved trafficking, transfection of target cells, epithelial reach, or a combination thereof as compared to a comparable delivery vehicle that lacks the charge separation. In some cases, the improvement is from about 1 fold, 50 fold, 99 fold, 148 fold, 197 fold, 246 fold, 295 fold, 344 fold, 393 fold, 442 fold, 491 fold, 540 fold, 589 fold, 638 fold, 687 fold, 736 fold, 785 fold, 834 fold, 883 fold, 932 fold, 981 fold, or up to about 1000 fold as compared to a comparable delivery vehicle that lacks the charge separation.

In some cases, a delivery vehicle can comprise any one of: MVL5/MC2/DSPC/Deoxycholate/DMG-PEG; MVL5/MC2/DSPC/Deoxycholate/DMPE-PEG; MVL5/CL1H6/DSPC/Deoxycholate/DMG-PEG; MVL5/CL4H6/DSPC/Deoxycholate/DMG-PEG; MVL5/MC2/DSPC/Chenodeoxycholate/DMG-PEG; MVL5/MC2/DMPC/Deoxycholate/DMG-PEG; MVL5/MC2/DMPC/Deoxycholate/DMPE-PEG; MVL5/CL1H6/DMPC/Deoxycholate/DMG-PEG; MVL5/MC2/DSPC/Deoxycholate/Lithocholate/DMG-PEG; MVL5/CL1H6/DSPC/Deoxycholate/Lithocholate/DMG-PEG; MVL5/MC2/DSPC/Alloisolithocholate/DMG-PEG; or MVL5/MC2/DSPC/Dehydrolithocholate/DMG-PEG.

A delivery vehicle can be generated using a variety of molar ratios. In some cases, a pharmaceutical formulation comprises MVL5, MC2, Deoxycholate, DSPC, and DMG-PEG at a molar ratio of about 0.96:0.96:2.592:3.168:0.0768:0.0384:0.0384. In some cases, the ratio of a cationic charge in a first locus to an anionic charge in a second locus at pH 7.4 is from about 0.25, 0.45, 0.65, 0.85, 1.05, 1.25, 1.45, 1.65, 1.85, 2.05, 2.25, 2.45, 2.65, or 2.85. In some cases, the ratio of a cationic charge in a first locus to an anionic charge in a second locus at pH 7.4 is from about 0.25 to about 1.05, 0.75 to about 1.25, 1.05 to about 1.45, or 0.85 to about 1.85. In another aspect, a ratio of a multivalent lipid to an ionizable cationic lipid in a delivery vehicle is from about (6%, 6.25%, 6.5%, 6.75%, 7%, 7.25%, 7.5%, 7.75%, or 8%) to (8%, 8.25%, 8.5%, 8.75%, 9%, 9.25%, 9.5%, 9.75%, 10%), (12%, 12.25%, 12.5%, 12.75%, or 13%) to (12%, 12.25%, 12.5%, 12.75%, or 13%), or (18%, 18.25%, 18.5%, 18.75%, 19%, 19.25%, 19.5%, 19.75%, 20%) to (6%, 6.25%, 6.5%, 6.75%, 7%, 7.25%, 7.5%, 7.75%, or 8%). In some aspects, a bile salt is at a concentration from about 10 mole %, 15 mole %, 20 mole %, 25 mole %, 30 mole %, 35 mole %, 40 mole %, 45 mole %, 50 mole %, 55 mole %, 60 mole %, 65 mole %, 70 mole %, 75 mole %, or about 80 mole %. In some cases, a bile salt is from about 10 mole % to 30 mole %, 20 mole % to 50 mole %, 30 mole % to 60 mole %, or 40 mole % to 80 mole %. Suitable alternate formulations can comprise multivalent lipid, ionizable cationic lipid, bile salt, structural lipid, and/or lipid-PEG at molar ratios from about 5%, 10%, 15%, 20%, 25%, 30%, 35%, 40%, 45%, 50%, 55%, 60%, 65%, 70%, 75%, 80%, 85%, 90%, or 95% more or less to those provided herein.

### Delivery Vehicle Stability

In some embodiments, the delivery vehicle stability can be increased with the incorporation of a bile acid or bile salt. The terms “bile acid,” “bile salt,” “bile acid/salt,” are, unless otherwise indicated, utilized interchangeably herein. Any reference to a bile acid used herein can include reference to a bile acid or a salt thereof. The term “bile acid” (and bile salt,” “bile acid/salt”) as used herein, can include steroid acids (and an anion thereof), and salts thereof, found in the bile of an animal (e.g., a human), including, by way of non-limiting example, cholic acid, cholate, deoxycholic acid, deoxycholate, hyodeoxycholic acid, hyodeoxycholate, glycocholic acid, glycocholate, taurocholic acid, taurocholate, chenodeoxycholic acid, chenodeoxycholate, lithocholic acid, lithocolate, and the like, or salts thereof. In some embodiments, a bile acid is ursodiol, isolithocholate, alloisolithocholate, dehydrolithochlate, or 5-beta-cholanic acid. Taurocholic acid and taurocholate are referred to herein as TCA. Any reference to a bile acid used herein can include reference to a bile acid, one and only one bile acid, one or more bile acids, or to at least one bile acid. Furthermore, pharmaceutically acceptable bile acid esters can be utilized as the “bile acids” described herein, e.g., bile acids conjugated to an amino acid (e.g., glycine or taurine). Other bile acid esters can include, e.g., substituted or unsubstituted alkyl ester, substituted or unsubstituted heteroalkyl esters, substituted or unsubstituted aryl esters, substituted or unsubstituted heteroaryl esters, or the like. For example, the term “bile acid” can include cholic acid conjugated with either glycine or taurine: glycocholate and taurocholate, respectively (and salts thereof). Any reference to a bile acid used herein can include reference to an identical compound naturally or synthetically prepared. Furthermore, it is to be understood that any singular reference to a component (bile acid or otherwise) used herein can include reference to one and only one, one or more, or at least one of such components. Similarly, any plural reference to a component used herein can include reference to one and only one, one or more, or at least one of such components, unless otherwise noted.

In some embodiments of the delivery vehicles herein, a bile salt can be cholic acid. In some embodiments, a bile salt can be deoxycholate. In some embodiments, the incorporation of bile salts can be cholic acid and deoxycholate. In some embodiments, the bile salt can comprise cholate, deoxycholate, their conjugates and derivatives, or combination thereof. In further embodiments, bile salts can be chenodeoxycholic, lithocholic, taurodeoxycholic, or combination thereof.

In some embodiments, the bile salt concentration in the lipid nanoparticles of a delivery vehicle (or in a composition comprising lipid nanoparticles) can comprise from about 80 mole % to about 10 mole %, such as from about 80 mole % to about 70 mole %, from about 65 mole % to about 55 mole %, from about 60 mole % to about 50%, from about 55 mole % to about 45 mole %, from about 50 mole % to about 40 mole %, from about 45 mole % to about 35 mole %, from about 40 mole % to about 30 mole %, from about 35 mole % to about 25 mole %, from about 30 mole % to about 20 mole %, from about 25 mole % to about 15 mole %, from about 20 mole % to about 10 mole %, from about 15 mole % to about 10 mole %, from about 60 mole % to about 20 mole %, from about 25.9 mole %, from about 30.4 mole %, about 34.9 mole %, from about 39.4 mole %, from about 37.1 mole %, from about 43.9 mole %, or about 45 mole %. In some cases, the bile salt concentration in a lipid nanoparticle of a delivery vehicle (or a composition comprising a lipid nanoparticle) can comprise about 5 mole %, 10 mole %, 15 mole %, 20 mole %, 25 mole %, 30 mole %, 35 mole %, 40 mole %, 45 mole %, 50 mole %, 55 mole %, 60 mole %, 65 mole %, 70 mole %, 75 mole %, 80 mole %, or 85 mole %.

An efficiency of cellular uptake with a structure, such as the compositions described herein having a bile salt included in the lipid nanoparticle of a delivery vehicle can permit efficient penetration and transit through the mucus layer to the target cells and thereby have an efficient uptake by the target cell(s), for example, uptake can be or can be about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9% of the total number of cells that are contacted. In some embodiments, the compositions can have a higher percent of cellular uptake as compared to a comparable delivery vehicle that does include a bile salt. The improvement can be from about 10%, 15%, 20%, 25%, 30%, 35%, 40%, 45%, 50%, 55%, 60%, 65%, 70%, or up to about 80% better. In some cases, an efficiency of transfection or integration of a polynucleic acid cargo delivered to a cell by a delivery vehicle composition as described herein can be from about 5%, 10%, 15%, 20%, 25%, 30%, 35%, 40%, 45%, 50%, 55%, 60%, or up to 65% better than a comparable delivery vehicle that does not include the bile salt, and additional features, such as an MPP and/or a particular composition of lipids. In some cases, an efficiency of transfection or integration of a polynucleic acid cargo delivered to a cell by a delivery vehicle composition as described herein can be from about 5%, 10%, 15%, 20%, 25%, 30%, 35%, 40%, 45%, 50%, 55%, 60%, or up to 65% better than a comparable delivery vehicle that does not include a bile salt.

In some embodiments, stability of the delivery vehicle can be measured by a bile salt stability assay, in a high bile salt mimicking environment. For example, bile salt stability can be measured by fluorescence spectroscopy, such as relative fluorescence of delivery vehicles containing varying concentrations of bile salts, in a Forster resonance energy transfer (FRET) assay. In some embodiments, the incorporated bile salt (s) can increase the stability of the delivery vehicle from about 80% to about 10%, such as about 80% to about 70%, about 65% to about 55%, about 60% to about 50%, about 55% to about 45%, about 50% to about 40%, about 45% to about 35%, about 40% to about 30%, about 35% to about 25%, about 30% to about 20%, about 25% to about 15%, about 20% to about 10%, about 15 mole % to about 10, about 60% to about 20%, about 25.9%, about 30.4%, about 34.9%, about 39.4%, about 37.1%, about 43.9%, or about 45%. In some embodiments, the incorporated bile salt (s) can increase the stability of the delivery vehicle as compared to a comparable delivery vehicle that lacks the bile salt. In some cases, a delivery vehicle that comprises a bile salt as provided herein can have improved trafficking, transfection of target cells, epithelial reach, or a combination thereof as compared to a comparable delivery vehicle that lacks the bile salt. In some cases, the improvement is from about 1 fold, 50 fold, 99 fold, 148 fold, 197 fold, 246 fold, 295 fold, 344 fold, 393 fold, 442 fold, 491 fold, 540 fold, 589 fold, 638 fold, 687 fold, 736 fold, 785 fold, 834 fold, 883 fold, 932 fold, 981 fold, or up to about 1000 fold as compared to a comparable delivery vehicle that lacks the bile salt. In some examples, the percent increase in stability can be measured by increased relative fluorescence units or relative luminescence units in an assay, such as FRET in vivo or ex vivo.

In some embodiments, a delivery vehicle of this disclosure can comprise a cationic lipid and a bile salt, wherein the lipid can be a saturated cationic lipid or an unsaturated cationic lipid, wherein the saturated cationic lipid may have a phase transition temperature that is at least about 20° C. In some embodiments, a delivery vehicle of this disclosure can comprise at least one saturated cationic lipid and at least a bile salt, wherein the at least one saturated cationic lipid can have a phase transition temperature of at least about 37° C. In some embodiments, the saturated cationic lipid has a phase transition temperature of at least about 20° C., 22° C., 24° C., 26° C., 28° C., 30° C., 32° C., 34° C., 36° C., 38° C., 40° C., 42° C., 44° C., 46° C., 48° C., 50° C., 52° C., 54° C., 56° C., 58° C., and/or up to about 60° C. For example, the saturated cationic lipid can have a phase transition temperature of 30° C.-60° C., 35° C.-60° C., 37° C.-60° C., 37° C.-55° C., 37° C.-50° C., 37° C.-45° C., or 37° C.-40° C. In some embodiments, a delivery vehicle of this disclosure can comprise at least one saturated cationic lipid and at least a bile salt, wherein the at least one saturated cationic lipid can have a phase transition temperature of at least about 37° C. The lipid delivery vehicle can further comprise a saturated non-cationic lipid. The saturated non-cationic lipid may have a phase transition temperature of at least about 20° C., 22° C., 24° C., 26° C., 28° C., 30° C., 32° C., 34° C., 36° C., 38° C., 40° C., 42° C., 44° C., 46° C., 48° C., 50° C., 52° C., 54° C., 56° C., 58° C., and/or up to about 60° C. For example, the saturated non-cationic lipid may have phase transition temperatures of about 30° C.-60° C., 35° C.-60° C., 37° C.-60° C., 37° C.-55° C., 37° C.-50° C., 37° C.-45° C., or 37° C.-40° C. The lipid delivery vehicle, in some cases, can further comprise a lipid conjugated to a hydrophilic polymer, such a polyethylene glycol (PEG). The delivery vehicle, in some cases, may be conjugated to at least one of: a cell penetrating peptide, a ligand, a mucus penetrating polymer, a peptide that enables mucus penetration, a cell penetrating peptide that is not substantially mucus adhesive, or any combinations thereof.

In some embodiments are provided a delivery vehicle comprising a cargo in a lipid structure, for example a lipid nanoparticle, and wherein the lipid nanoparticle comprises a bile salt and at least one of: (a) a saturated cationic lipid that has a phase transition temperature of at least about 37° C., and a non-cationic lipid; (b) a saturated cationic lipid, an unsaturated cationic lipid, a non-cationic lipid, wherein the unsaturated cationic lipid, the non-cationic lipid, or the unsaturated cationic lipid and the non-cationic lipid, have a phase transition temperature of at least about 37° C.; or (c) a multivalent cationic lipid, a non-cationic lipid, wherein the multivalent cationic lipid, the non-cationic lipid, or the multivalent cationic lipid and the non-cationic lipid have a phase transition temperature of at least about 37° C., wherein the delivery vehicle is stable in a high bile salt environment, compared to an otherwise identical delivery vehicle that (i) does not comprise a lipid nanoparticle comprising the bile salt and at least one of (a), (b), or (c); (ii) comprises a lipid nanoparticle comprising at least one of (a), (b), or (c), but does not comprise the bile salt, or (iii) comprises the bile salt but does not comprise at least one of (a), (b), or (c). The saturated cationic lipid, unsaturated cationic lipid, non-cationic lipid, and/or multivalent cationic lipid may have a phase transition temperature of at least about 20° C., 22° C., 24° C., 26° C., 28° C., 30° C., 32° C., 34° C., 36° C., 38° C., 40° C., 42° C., 44° C., 46° C., 48° C., 50° C., 52° C., 54° C., 56° C., 58° C., and/or up to about 60° C. For example, the saturated cationic lipid, unsaturated cationic lipid, non-cationic lipid, and/or multivalent cationic lipid may have phase transition temperatures of about 30° C.-60° C., 35° C.-60° C., 37° C.-60° C., 37° C.-55° C., 37° C.-50° C., 37° C.-45° C., or 37° C.-40° C.

In some embodiments are provided a delivery vehicle comprising a cargo and a lipid structure, such as a lipid nanoparticle, wherein the lipid nanoparticle comprises a bile salt and at least one of: (a) a saturated cationic lipid that has a phase transition temperature of at least about 37° C.; (b) a saturated cationic lipid, an unsaturated cationic lipid and a non-cationic lipid, wherein the unsaturated cationic lipid, the non-cationic lipid, or the unsaturated cationic lipid and the non-cationic lipid, have a phase transition temperature of at least about 37° C.; or (c) a multivalent cationic lipid and a non-cationic lipid, wherein the multivalent cationic lipid, the non-cationic lipid, or the multivalent cationic lipid and the non-cationic lipid have a phase transition temperature of at least about 37° C., wherein the delivery vehicle demonstrates an increased stability in a solution containing at least about 5 g/L of cholic acid and deoxycholate, compared to an otherwise identical lipid nanoparticle (i) does not comprise a lipid nanoparticle comprising the bile salt and at least one of (a), (b), or (c); (ii) comprises a lipid nanoparticle comprising at least one of (a), (b), or (c), but does not comprise the bile salt, or (iii) comprises the bile salt but does not comprise at least one of (a), (b), or (c), wherein the stability is measured by relative fluorescence intensity of a fluorescent lipid incorporated into the lipid nanoparticle, in a Forster resonance energy transfer (FRET) assay. In some cases, the delivery vehicle demonstrates an increased stability in a solution containing at least about 0.5 g/L, 1 g/L, 5 g/L, 7 g/L, 9 g/L, 11 g/L, 13 g/L, 15 g/L, 17 g/L, 19 g/L, 21 g/L, 23 g/L, or up to about 25 g/L of bile acid, for example, a mixture of about 40%, 45%, 50%, or up to about 55% cholic acid and about 40%, 45%, 50%, 55%, or up to about 60% deoxycholate, compared to an otherwise identical delivery vehicle that (i) does not comprise the bile salt, wherein the stability is measured by relative fluorescence intensity of a fluorescent lipid incorporated into the lipid nanoparticle, in a Forster resonance energy transfer (FRET) assay.

In some embodiments are provided a delivery vehicle comprising (i) a cargo and (ii) a lipid structure, such as a lipid nanoparticle, wherein the lipid nanoparticle comprises at least one saturated cationic lipid and a bile salt, wherein the at least one saturated cationic lipid has a phase transition temperature of at least about 37° C. In some embodiments are provided a delivery vehicle comprising (i) a cargo and a (ii) lipid nanoparticle, wherein the lipid nanoparticle comprises at least one saturated lipid, at least one unsaturated cationic lipid, and a bile salt, wherein the concentration of the at least one unsaturated cationic lipid in the lipid nanoparticle is less than 50 mole %.

Exemplary delivery vehicles are described herein and provided for example at Table 1, Table 2, Table 3, and Table 4. Any one of the delivery vehicles exemplified in Table 1-Table 4 can be further modified. For example, additional lipids, cargo, modifications to, additions to, subtractions to, can be made. In some cases, any one of the delivery vehicles in Table 1 can further comprise lipid-PEG.

TABLE 1
Exemplary delivery vehicles for delivering cargo. Abbreviations:
BS: bile salt, SC: saturated cationic, UC: unsaturated cationic,
SN: saturated non-cationic, UN: unsaturated non-cationic,
MV: multivalent cationic, SMV: Multivalent cationic saturated,
UMV: Multivalent cationic unsaturated. Where “x”
appears in the formula, this denotes at least one (i.e.,
x is equal to or greater than 1).
SN:UC:BS
SN:SMV:BS
SN:UMV:BS
SN:[(UC)x + (MV)x + (UN)x + (SN)x]:BS
SC:BS
SC:UN:BS
SC:UC:BS
SC:UMV:BS
SC:SN:BS
SC:[(UC)x + (MV)x + (SN)x + (SC)x]:BS
SMV:BS
SMV:UN:BS
SMV:SN:BS
SMV:[(UC)x + (MV)x + (SN)x + (SC)x]:BS
UMV:UN:BS

### Lipids for Use in Delivery Vehicles

The delivery vehicles herein, including those with a cargo, include one or more lipids such as in a lipid nanoparticle. In some embodiments, the lipid nanoparticle includes at least one saturated lipid, at least one of an unsaturated cationic lipid or an unsaturated non-cationic lipid, and a bile salt. In some embodiments, the lipid nanoparticle includes at least one saturated lipid, where the saturated lipid comprises a saturated cationic lipid that has a phase transition temperature of at least about 37° C. or a saturated non-cationic lipid that has a phase transition temperature of at least about 37° C. In some aspects, the lipid nanoparticle further includes at least one of: a non-cationic lipid, a multivalent cationic lipid, a permanently charged cationic lipid, or any combinations thereof. In some embodiments, the lipid nanoparticle comprises a bile salt and a multivalent cationic lipid and a non-cationic lipid, where the multivalent cationic lipid, the non-cationic lipid, or the multivalent cationic lipid and the non-cationic lipid have a phase transition temperature of at least about 37° C. In some embodiments, the lipid nanoparticle comprises a bile salt and a saturated cationic lipid that has a phase transition temperature of at least about 37° C., and a non-cationic lipid. In some embodiments, the lipid nanoparticle comprises a bile salt and a saturated cationic lipid, an unsaturated cationic lipid, and a non-cationic lipid, wherein the unsaturated cationic lipid, the non-cationic lipid, or the unsaturated cationic lipid and the non-cationic lipid have a phase transition temperature of at least about 37° C. In some embodiments, the delivery vehicle has a first locus that is positively charged at a pH between about 5.5 and 8.0, and a second locus that is negatively charged at a pH between about 5.5 and 8.0, wherein the first and second loci are separated such that the positive and negative charges are not interspersed, and wherein one or both loci contain a lipid. In some embodiments, the first locus comprises an unsaturated or short-tail lipid, such as a cationic or ionizable cationic lipid, for example, a multivalent cationic lipid or a monovalent cationic lipid.

In an aspect, a cationic lipid for use in the lipid nanoparticles of the delivery vehicles herein can include N-[1-(2,3-dioleyloxy)propyl]-N,N,N-trimethylammonium chloride (DOTMA), [1,2-bis(oleoyloxy)-3-(trimethylammonio)propane] (DOTAP), dimethyldioctadecylammonium (DDA), 30[N—(N′,N′-dimethylaminoethane)-carbamoyl]cholesterol (DC-Chol), and dioctadecylamidoglycylspermine (DOGS). Dioleoylphosphatidylethanolamine (DOPE), polyethyleneimines (PEI), a neutral lipid, may often be used in conjunction with cationic lipids because of its membrane destabilizing effects at low pH, which can aide in endolysosomal escape. In some embodiments, a saturated cationic lipid can be employed in a delivery vehicle provided herein. A saturated cationic lipid can have a positive charge at pH 4, or at a pH greater than pH 4. In some embodiments, the saturated cationic lipid can comprise at least one of: 1,2-dialkyl-sn-glycero-3-ethylphosphocholine, 1,2-dialkyl-3-dimethylammonium-propane, 1,2-dialkyl-3-trimethylammonium-propane, 1,2-di-O-alkyl-3-trimethylammonium propane, 1,2-dialkyloxy-3-dimethylaminopropane, N,N-dialkyl-N,N-dimethylammonium, N-(4-carboxybenzyl)-N,N-dimethyl-2,3-bis(alkyloxy)propan-1-aminium, 1,2-dialkyl-sn-glycero-3-[(N-(5-amino-1-carboxypentyl)iminodiacetic acid)succinyl], N1-[2-((1S)-1-[(3-aminopropyl)amino]-4-[di(3-amino-propyl)amino]butylcarboxamido)ethyl]-3,4-di[alkyl]-benzamide, or any combinations thereof. In examples where the saturated cationic lipid comprises an alkyl, the alkyl can be a conjugated derivative of at least one of: myristoyl, pentadecanoyl, palmitoyl, heptadecanoyl, stearoyl, lauroyl, tridecanoyl, nonadecanoyl, arachidoyl, heneicasnoyl, behenoyl, tricosanoyl, lignoceroyl, or any combinations thereof. In some embodiments, the saturated cationic lipid can comprise at least one of: saturated cationic lipid that has a phase transition temperature of at least about 37° C. comprises at least one of: 1,2-stearoyl-3-trimethylammonium-propane (DSTAP), 1,2-dipalmitoyl-3-trimethylammonium-propane (DPTAP), 1,2-Distearoyl-3-Dimethylammonium-Propane (DSDAP), or any combinations thereof. In an aspect, a cationic lipid may be in a gel phase of a lipid structure and an anionic lipid may be in a liquid phase.

In some embodiments, a lipid nanoparticle of a delivery vehicle can comprise at least one unsaturated cationic lipid. In some embodiments, the unsaturated cationic lipid can have a positive charge at about pH 4, or at a pH greater than about pH 4 and less than about pH 8. In some embodiments, the unsaturated cationic lipid can comprise at least one of: 1,2-dialkyl-sn-glycero-3-ethylphosphocholine, 1,2-dialkyl-3-dimethylammonium-propane, 1,2-dialkyl-3-trimethylammonium-propane, 1,2-di-O-alkyl-3-trimethylammonium propane, 1,2-dialkyloxy-3-dimethylaminopropane, N,N-dialkyl-N,N-dimethylammonium, N-(4-carboxybenzyl)-N,N-dimethyl-2,3-bis(alkyloxy)propan-1-aminium, 1,2-dialkyl-sn-glycero-3-[(N-(5-amino-1-carboxypentyl)iminodiacetic acid)succinyl], N1-[2-((1S)-1-[(3-aminopropyl)amino]-4-[di(3-amino-propyl)amino]butylcarboxamido)ethyl]-3,4-di[alkyl]-benzamide, 1,2-Dialkyloxy-N,N-dimethylaminopropane, 4-(2,2-diocta-9,12-dienyl-[1,3]dioxolan-4-ylmethyl)-dimethylamine, O-alkyl ethylphosphocholines, MC3, MC2, MC4, 3β-[N—(N′,N′-dimethylaminoethane)-carbamoyl]cholesterol, N4-Cholesteryl-Spermine, or salts thereof, or any combinations thereof. In examples where the unsaturated cationic lipid comprises an alkyl, the alkyl can be a conjugated derivative of at least one of: oleic acid, elaidic acid, gondoic acid, erucic acid, nervonic acid, mead acid, paullinic acid, vaccenic acid, palmitoleic acid, Docosatetraenoic acid, Arachidonic acid, Dihomo-γ-linolenic acid, γ-Linolenic acid, linolelaidic acid, linoleic acid, Docosahexaenoic acid, Eicosapentaenoic acid, Stearidonic acid, α-Linolenic acid, or any combinations thereof. In some embodiments, the unsaturated cationic lipid can comprise at least one of: 1,2-Dialkyloxy-N,N-dimethylaminopropane, 4-(2,2-diocta-9,12-dienyl-[1,3]dioxolan-4-ylmethyl)-dimethylamine, O-alkyl ethylphosphocholines, MC3, MC2, MC4, 3ß-[N—(N′,N′-dimethylaminoethane)-carbamoyl]cholesterol, N4-Cholesteryl-Spermine, or salts thereof, or any combinations thereof. In some cases, a lipid can comprise or can be 7-(4-(dimethylamino)butyl)-7-hydroxytridecane-1,13-diyl dioleate (CL1H6), CL1A6, CL1A6, CL3A6, CL4A6, CL5A6, CL6A6, CL7A6, CL8A6, CL9A6, CL10A6, CL11A6, CL12A6, CL13A6, CL14A6, CL15A6, YSK12-C4, as described in US20200129431A1 and Sato Yet al. Understanding structure-activity relationships of pH-sensitive cationic lipids facilitates the rational identification of promising lipid nanoparticles for delivering siRNAs in vivo. J Control Release. 2019; 295:140-152 both herein incorporated by reference. In an aspect, a cationic lipid may be in a liquid phase of a lipid structure and an anionic lipid may be in a gel phase or solid phase of a lipid structure.

In some cases, a lipid nanoparticle of a delivery vehicle may comprise a multivalent cationic lipid. A multivalent cationic lipid can be selected from: N 1-[2-((1 S)-1-[(3-aminopropyl)amino]-4-[di(3-amino-propyl)amino]butylcarboxamido)ethyl]-3,4-di[oleyloxy]-benzamide (MVL5) a salt thereof, and any combination thereof. In an aspect, a delivery vehicle provided herein can be generated using MVL5. In an aspect, MVL5, GL67, or a combination thereof are in a liquid phase of a delivery vehicle. Any of the multivalent cationic lipids provided herein can be incorporated in a provided vehicle or particle at less than about 50 mole %, 48 mole %, 46 mole %, 44 mole %, 42 mole %, 40 mole %, 38 mole %, 36 mole %, 34 mole %, 32 mole %, 30 mole %, 28 mole %, 26 mole %, 24 mole %, 22 mole %, 20 mole %, 18 mole %, 16 mole %, 14 mole %, 12 mole %, 10 mole %, 8 mole %, 6 mole %, 4 mole %, 2 mole %, or 0 mole %. Any of the multivalent cationic lipids provided herein can be incorporated in a provided vehicle or particle at about 50 mole %, 48 mole %, 46 mole %, 44 mole %, 42 mole %, 40 mole %, 38 mole %, 36 mole %, 34 mole %, 32 mole %, 30 mole %, 28 mole %, 26 mole %, 24 mole %, 22 mole %, 20 mole %, 18 mole %, 16 mole %, 14 mole %, 12 mole %, 10 mole %, 8 mole %, 6 mole %, 4 mole %, 2 mole %, or 0 mole %. In some embodiments, the multivalent cationic lipids provided herein can be incorporated in a provided vehicle or particle in a concentration of 5-50 mole %, 5-40 mole %, 5-30 mole %, 5-25 mole %, 5-20 mole %, 5-15 mole %, 10-50 mole %, 10-40 mole %, 10-30 mole %, 10-25 mole %, 15-50 mole %, 15-40 mole %, 15-30 mole % and 15-25 mole 0.

In some embodiments, a lipid nanoparticle of a delivery vehicle provided herein can also comprise an anionic lipid. An anionic lipid can contain any of a wide range of fatty acid chains in the hydrophobic region. The specific fatty acids incorporated are responsible for the fluidic characteristics of the lipid structure in terms of phase behavior and elasticity. In some cases, divalent cations can be incorporated into an anionic lipid structure to enable the condensation of nucleic acids prior to envelopment by anionic lipids. Several divalent cations can be used in anionic lipoplexes such as Ca²⁺, Mg²⁺, Mn²⁺, and Ba²⁺. In some cases, Ca²⁺ can be utilized in an anionic lipid structure. Suitable anionic lipids include but are not limited to: phosphatidylglycerol, a cardiolipin, a diacylphosphatidylserine, a diacylphosphatidic acid, a N-dodecanoyl phosphatidylethanolamine, a N-succinyl phosphatidylethanolamine, a N-glutarylphosphatidylethanolamine, a lysylphosphatidylglycerol, a palmitoyloleyolphosphatidylglyeerol (POPG), or any combinations thereof.

In some embodiments, the anionic lipid in the lipid nanoparticles comprises at least one of phosphatidylglycerol, cardiolipin, dialkylphosphatidylserine, dialkylphosphatidic acid, N-dodecanoyl phosphatidylethanolamine, N-succinyl phosphatidylethanolamine, N-glutarylphosphatidylethanolamine, lysylphosphatidylglycerol, palmitoyloleyolphosphatidylglycerol (POPG), glycerophosphoinositol monophosphate, glycerophosphoinositol bisphosphate, glycerophosphoinositol trisphosphate, glycerophosphate, a glyceropyrophosphate, glycerophosphoglycerophosphoglycerol, cytidine-5′-diphosphate-glycerols, glycosylglycerophospholipid, a glycerophosphoinositolglycan, 1,2-dialkyl-sn-glycero-3-Phosphate, 1,2-dialkyl-sn-glycero-3-phosphomethanol, 1,2-dialkyl-sn-glycero-3-phosphoethanol, 1,2-dialkyl-sn-glycero-3-phosphopropanol, and/or 1,2-dialkyl-sn-glycero-3-phosphobutanol. In some aspects where the anionic lipid is conjugated to an alkyl and the anionic lipid is present in the liquid phase, the alkyl is a conjugated derivative of at least one of oleic acid, elaidic acid, gondoic acid, erucic acid, nervonic acid, mead acid, paullinic acid, vaccenic acid, palmitoleic acid, Docosatetraenoic acid, Arachidonic acid, Dihomo-γ-linolenic acid, γ-Linolenic acid, linolelaidic acid, linoleic acid, Docosahexaenoic acid, Eicosapentaenoic acid, Stearidonic acid, α-Linolenic acid, or salts thereof, or any combinations thereof. In other cases, the alkyl is a conjugated derivative of at least one myristic acid, pentadecylic acid, palmitic acid, heptadecanoic acid, stearic acid, lauric acid, tridecylic acid, nonadecylic acid, arachidic acid, heneicosylic acid, behenic acid, tricosylic acid, lignoceric acid, and/or salts thereof, or any combinations thereof. In the above, if the alkyl has a phase transition temperature of >37 C it is considered to be in the gel phase otherwise it is present in the liquid phase.

In an aspect, an anionic lipid can be a saturated lipid with a phase transition temperature above 37 C, such a lipid can be used in a solid phase and a cationic lipid in a liquid phase. In a case when an anionic lipid is unsaturated or a short chain lipid with a transition temperature below 37 C then it may be employed in a liquid phase and a cationic lipid can be used in a gel or solid phase.

In an aspect, the concentration of at least one unsaturated cationic lipid and/or unsaturated non-cationic lipid in a lipid nanoparticle can be less than 50 mole %, 45 mole %, 40 mole %, 35 mole %, 30 mole %, 25 mole %, 20 mole %, 15 mole %, 10 mole %, 5 mole %, or 2 mole % of the total lipid concentration of the lipid nanoparticle. In some embodiments, the concentration of at least one unsaturated cationic lipid and/or unsaturated non-cationic lipid in a lipid nanoparticle can be about 50 mole %, 45 mole %, 40 mole %, 35 mole %, 30 mole %, 25 mole %, 20 mole %, 15 mole %, 10 mole %, 5 mole %, or 2 mole % of the total lipid concentration of the lipid nanoparticle. In some embodiments, the concentration of at least one unsaturated cationic lipid and/or unsaturated non-cationic lipid in a lipid nanoparticle can be 5-50 mole %, 5-40 mole %, 5-30 mole %, 5-25 mole %, 5-20 mole %, 5-15 mole %, 10-50 mole %, 10-40 mole %, 10-30 mole %, 10-25 mole %, 15-50 mole %, 15-40 mole %, 15-30 mole % and 15-25 mole %.

In some cases, a delivery vehicle may comprise a high temperature phase transition lipid, for example, a high temperature phase transition neutral lipid such as DSPC, and a bile salt such as deoxycholate, cholic acid or a conjugate thereof. Deoxycholate can serve as a solid phase (gel phase) where deoxycholate provides the negative charge. On the same delivery vehicle, a cationic lipid can be present as unsaturated or a short tail lipid and can be present in the liquid phase. Multivalent cationic lipids, like MVL5, can be used to create enough positive to negative charge ratio to provide the system with a balance of attraction and repulsion thereby generating a delivery vehicle containing a charge separation.

In some embodiments, a delivery vehicle can further comprise a conjugated lipid, wherein the conjugated lipid can comprise a lipid conjugated to a stabilizing component. In some embodiments, the stabilizing component can comprise a hydrophilic polymer. In some embodiments, the hydrophilic polymer can comprise polyethylene glycol, a poly (2-alkyl-2-oxazoline), a polyvinyl alcohol, or any combinations thereof. In some embodiments, the hydrophilic polymer can comprise a molecular weight from at least about 500 Da to about 500 kDa, from at least about. In some embodiments, the hydrophilic polymer can comprise the polyethyleneglycol (PEG), and wherein the conjugated lipid comprises a pegylated lipid. In some embodiments, the pegylated lipid can comprise DSPE-PEG, DSG-PEG, DPG-PEG, DAG-PEG, DMG-PEG, DPPE-PEG, DMPE-PEG, or any combinations thereof.

In some cases, the concentration of the conjugated lipid can be less than about or more than about: 0 mole %, 0.5 mole %, 1 mole %, 1.5 mole %, 2 mole %, 2.5 mole %, 3 mole %, 3.5 mole %, 4 mole %, 4.5 mole %, 5 mole %, 5.5 mole %, 6 mole %, 6.5 mole %, 7 mole %, 7.5 mole %, 8 mole %, 8.5 mole %, 9 mole %, 9.5 mole %, 10 mole %, 10.5 mole %, 11 mole %, 11.5 mole %, 12 mole %, 12.5 mole %, 13 mole %, 13.5 mole %, 14 mole %, 14.5 mole %, 15 mole %, 15.5 mole %, 16 mole %, 16.5 mole %, 17 mole %, 17.5 mole %, 18 mole %, 18.5 mole %, 19 mole %, 19.5 mole %, 20 mole %, 20.5 mole %, 21 mole %, 21.5 mole %, 22 mole %, 22.5 mole %, 23 mole %, 23.5 mole %, 24 mole %, 24.5 mole %, 25 mole %, 25.5 mole %, 26 mole %, 26.5 mole %, 27 mole %, 27.5 mole %, 28 mole %, 28.5 mole %, 29 mole %, 29.5 mole %, or 30 mole %. In some cases, a concentration of the conjugated lipid is from about 0.5 mole % to about 20 mole %, 0.5 mole % to about 5 mole %, 0.5 mole % to about 10 mole %, 5 mole % to about 10 mole %, or 10 mole % to about 20 mole %.

In some cases, bile salts can be used as an anionic component in a delivery vehicle. In other cases, non-bile salts can be used as an anionic component. In some embodiments, the delivery vehicle stability can be increased with the incorporation of the bile salts (also referred to herein as bile acids), such as cholic acid, cholate, deoxycholic acid, deoxycholate, hyodeoxycholic acid, hyodeoxycholate, glycocholic acid, glycocholate, taurocholic acid, taurocholate, chenodeoxycholic acid, chenodeoxycholate, lithocholic acid, and lithocolate. In some embodiments, a bile salt can be cholic acid. In further embodiments, a bile salt can be deoxycholate. In some embodiments, the incorporation of bile salts can be cholic acid and deoxycholate. In some embodiments, stability of the delivery vehicle can be measured by a bile salt stability assay, in a high bile salt mimicking environment. For example, bile salt stability can be measured by fluorescence spectroscopy, such as relative fluorescence of delivery vehicles containing varying concentrations of bile salts, in a Forster resonance energy transfer (FRET) assay. In some embodiments, the incorporated bile salt (s) can increase the stability of the delivery vehicle from about 80% to about 10%, such as about 80% to about 70%, about 65% to about 55%, about 60% to about 50%, about 55% to about 45%, about 50% to about 40%, about 45% to about 35%, about 40% to about 30%, about 35% to about 25%, about 30% to about 20%, about 25% to about 15%, about 20% to about 10%, about 15% to about 10, about 60% to about 20%, about 25.9%, about 30.4%, about 34.9%, about 39.4%, about 37.1%, about 43.9%, or about 45%. In some examples, the percent increase in stability can be measured by increased relative fluorescence units or relative luminescence units in an assay, such as FRET.

In some cases, a delivery vehicle provided herein can comprise at least one of a multivalent lipid, cationic lipid, structural lipid, bile salt, or lipid-PEG. Any or all of the lipids provided herein can be formulated at any mole % for example, including but not limited to: 0 mole %, 0.5 mole %, 1 mole %, 1.5 mole %, 2 mole %, 2.5 mole %, 3 mole %, 3.5 mole %, 4 mole %, 4.5 mole %, 5 mole %, 5.5 mole %, 6 mole %, 6.5 mole %, 7 mole %, 7.5 mole %, 8 mole %, 8.5 mole %, 9 mole %, 9.5 mole %, 10 mole %, 10.5 mole %, 11 mole %, 11.5 mole %, 12 mole %, 12.5 mole %, 13 mole %, 13.5 mole %, 14 mole %, 14.5 mole %, 15 mole %, 15.5 mole %, 16 mole %, 16.5 mole %, 17 mole %, 17.5 mole %, 18 mole %, 18.5 mole %, 19 mole %, 19.5 mole %, 20 mole %, 20.5 mole %, 21 mole %, 21.5 mole %, 22 mole %, 22.5 mole %, 23 mole %, 23.5 mole %, 24 mole %, 24.5 mole %, 25 mole %, 25.5 mole %, 26 mole %, 26.5 mole %, 27 mole %, 27.5 mole %, 28 mole %, 28.5 mole %, 29 mole %, 29.5 mole %, 30 mole %, 30.5 mole %, 31 mole %, 31.5 mole %, 32 mole %, 32.5 mole %, 33 mole %, 33.5 mole %, 34 mole %, 34.5 mole %, 35 mole %, 35.5 mole %, 36 mole %, 36.5 mole %, 37 mole %, 37.5 mole %, 38 mole %, 38.5 mole %, 39 mole %, 39.5 mole %, 40 mole %, 40.5 mole %, 41 mole %, 41.5 mole %, 42 mole %, 42.5 mole %, 43 mole %, 43.5 mole %, 44 mole %, 44.5 mole %, 45 mole %, 45.5 mole %, 46 mole %, 46.5 mole %, 47 mole %, 47.5 mole %, 48 mole %, 48.5 mole %, 49 mole %, 49.5 mole %, 50 mole %, 50.5 mole %, 51 mole %, 51.5 mole %, 52 mole %, 52.5 mole %, 53 mole %, 53.5 mole %, 54 mole %, 54.5 mole %, 55 mole %, 55.5 mole %, 56 mole %, 56.5 mole %, 57 mole %, 57.5 mole %, 58 mole %, 58.5 mole %, 59 mole %, 59.5 mole %, 60 mole %, 60.5 mole %, 61 mole %, 61.5 mole %, 62 mole %, 62.5 mole %, 63 mole %, 63.5 mole %, 64 mole %, 64.5 mole %, 65 mole %, 65.5 mole %, 66 mole %, 66.5 mole %, 67 mole %, 67.5 mole %, 68 mole %, 68.5 mole %, 69 mole %, 69.5 mole %, 70 mole %, 70.5 mole %, 71 mole %, 71.5 mole %, 72 mole %, 72.5 mole %, 73 mole %, 73.5 mole %, 74 mole %, 74.5 mole %, 75 mole %, 75.5 mole %, 76 mole %, 76.5 mole %, 77 mole %, 77.5 mole %, 78 mole %, 78.5 mole %, 79 mole %, 79.5 mole %, or 80 mole %.

In some embodiments, the delivery vehicles herein can include additional components. For example, a lipid structure for a delivery vehicle can include a lipid bilayer. In certain cases, a lipid bilayer can be generated of one or more compositions selected from the group consisting of a phospholipid, a phosphatidyl-choline, a phosphatidyl-serine, a phosphatidyl-diethanolamine, a phosphatidylinosite, a sphingolipid, and an ethoxylated sterol, or mixtures thereof. In illustrative examples of such embodiments, the phospholipid can be a lecithin; the phosphatidylinosite can be derived from soy, rape, cotton seed, egg and mixtures thereof; the sphingolipid can be ceramide, a cerebroside, a sphingosine, and a sphingomyelin, and a mixture thereof, the ethoxylated sterol can be phytosterol, PEG-(polyethyleneglycol)-5 rapeseed sterol. In certain embodiments, the phytosterol comprises a mixture of at least two of the following compositions: sistosterol, camposterol and stigmasterol. In still other embodiments, a lipid layer can be comprised of one or more phosphatidyl groups selected from the group comprising phosphatidyl choline, phosphatidylethanolamine, phosphatidyl-serine, phosphatidyl-inositol, lyso-phosphatidyl-choline, lyso-phosphatidyl-ethanolamnine, lyso-phosphatidyl-inositol or lyso-phosphatidyl-inositol. In other cases, a lipid bilayer can be comprised of phospholipid selected from a monoacyl or diacylphosphoglyceride. In still other cases, a lipid bilayer can be comprised of one or more phosphoinositides selected from the group comprising phosphatidyl-inositol-3-phosphate (PI-3-P), phosphatidyl-inositol-4-phosphate (PI-4-P), phosphatidyl-inositol-5-phosphate (PI-5-P), phosphatidyl-inositol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-inositol-3,4,5-triphosphate (PI-3,4,5-P3), lysophosphatidyl-inositol-3-phosphate (LPI-3-P), lysophosphatidyl-inositol-4-phosphate (LPI-4-P), lysophosphatidyl-inositol-5-phosphate (LPI-5-P), lysophosphatidyl-inositol-3,4-diphosphate (LPI-3,4-P2), lysophosphatidyl-inositol-3,5-diphosphate (LPI-3,5-P2), lysophosphatidyl-inositol-4,5-diphosphate (LPI-4,5-P2), and lysophosphatidyl-inositol-3,4,5-triphosphate (LPI-3,4,5-P3), phosphatidyl-inositol (PI), or lysophosphatidyl-inositol (LPI).

Lipid structures used as delivery vehicles may be modified. A modification can be a surface modification. A surface modification can enhance an average rate at which a lipid structure moves in mucus compared to a comparable lipid structure. A comparable lipid structure may not be surface modified, or a comparable lipid structure may be modified with a polyethylene glycol (PEG) polymer. A modification can facilitate protection from degradation in vivo. A modification may also assist in trafficking of a lipid structure. For example, a modification may allow a lipid structure to traffic within a gastrointestinal (GI) track with an acidic pH due to pH sensitive modifications. A surface modification can also improve an average rate at which a lipid structure moves in mucous. For example, a modification may enhance a rate by 1×, 2×, 3×, 4×, 5×, 6×, 7×, 8×, 9×, 10×, 20×, 30×, 40×, 50×, 60×, 70×, 80×, 90×, 100×, 300×, 500×, 700×, 900×, or up to about 1000× when compared to a comparable lipid structure without a modification or a lipid structure with a modification comprising PEG. In some cases, a modification to a lipid structure occurs via a bond. A bond can be covalent, noncovalent, polar, ionic, hydrogen, or any combination thereof. A bond can be considered an association of two groups or portions of groups. For example, a lipid structure can be bonded to a PEG via a linker comprising a covalent bond. In some cases, a bond can occur between two adjacent groups. Bonds can be dynamic. A dynamic bond can occur when one group temporarily associates with a second group. For example, a polynucleic acid in suspension within a liposome may bond with portions of a lipid bilayer during its suspension.

In some cases, a modification can be a polyethylene glycol (PEG) addition. Methods of modifying lipid structure surfaces with PEG can include its physical adsorption onto a lipid structure surface, its covalent attachment onto a lipid structure, its coating onto a lipid structure, or any combination thereof. In some cases, PEG can be covalently attached to a lipid particle before a lipid structure is formed. A variety of molecular weights of PEG may be used. PEG can range from about 10 to about 100 units of an ethylene PEG component which may be conjugated to phospholipid through an amine group comprising or comprising about 1% to about 20%, preferably about 5% to about 15%, about 10% by weight of the lipids which are included in a lipid structure.

In some cases, a lipid structure can comprise a phosphatidylcholine. Exemplary phosphatidylcholines include but are not limited to dilauroyl phophatidylcholine, dimyristoylphophatidylcholine, dipalmitoylphophatidylcholine, distearoylphophatidyl-choline, diarachidoylphophatidylcholine, dioleoylphophatidylcholine, dilinoleoyl-phophatidylcholine, dierucoylphophatidylcholine, palmitoyl-oleoyl-phophatidylcholine, egg phosphatidylcholine, myristoyl-palmitoylphosphatidylcholine, palmitoyl-myristoyl-phdsphatidylcholine, myristoyl-stearoylphosphatidylcholine, palmitoyl-stearoylphosphatidylcholine, stearoyl-palmitoylphosphatidylcholine, stearoyl-oleoyl-phosphatidylcholine, stearoyl-linoleoylphosphatidylcholine and palmitoyl-linoleoylphosphatidylcholine. Asymmetric phosphatidylcholines can be referred to as 1-acyl, 2-acyl-sn-glycero-3-phosphocholines, wherein the acyl groups are different from each other. Symmetric phosphatidylcholines can be referred to as 1,2-diacyl-sn-glycero-3-phosphocholines. As used herein, the abbreviation “PC” refers to phosphatidylcholine. The phosphatidylcholine 1,2-dimyristoyl-sn-glycero-3-phosphocholine can be abbreviated herein as “DMPC.” The phosphatidylcholine 1,2-dioleoyl-sn-glycero-3-phosphocholine can be abbreviated herein as “DOPC.” The phosphatidylcholine 1,2-dipalmitoyl-sn-glycero-3-phosphocholine can be abbreviated herein as “DPPC.” In general, saturated acyl groups found in various lipids include groups having the names propionyl, butanoyl, pentanoyl, caproyl, heptanoyl, capryloyl, nonanoyl, capryl, undecanoyl, lauroyl, tridecanoyl, myristoyl, pentadecanoyl, palmitoyl, phytanoyl, heptadecanoyl, stearoyl, nonadecanoyl, arachidoyl, heneicosanoyl, behenoyl, trucisanoyl and lignoceroyl. The corresponding IUPAC names for saturated acyl groups are trianoic, tetranoic, pentanoic, hexanoic, heptanoic, octanoic, nonanoic, decanoic, undecanoic, dodecanoic, tridecanoic, tetradecanoic, pentadecanoic, hexadecanoic, 3,7,11,15-tetramethylhexadecanoic, heptadecanoic, octadecanoic, nonadecanoic, eicosanoic, heneicosanoic, docosanoic, trocosanoic and tetracosanoic. Unsaturated acyl groups found in both symmetric and asymmetric phosphatidylcholines include myristoleoyl, palmitoleyl, oleoyl, elaidoyl, linoleoyl, linolenoyl, eicosenoyl and arachidonoyl. The corresponding IUPAC names for unsaturated acyl groups are 9-cis-tetradecanoic, 9-cis-hexadecanoic, 9-cis-octadecanoic, 9-trans-octadecanoic, 9-cis-12-cis-octadecadienoic, 9-cis-12-cis-15-cisoctadecatrienoic, 11-cis-eicosenoic and 5˜cis-8-cis-11-cis-14-cis-eicosatetraenoic. Exemplary phosphatidylethanolamines include dimyristoyl-phosphatidylethanolamine, dipalmitoyl-phosphatidylethanolamine, distearoyl phosphatidylethanolamine, dioleoyl-phosphatidylethanolamine and egg phosphatidylethanolamine. Phosphatidylethanolamines may also be referred to under IUPAC naming systems as 1,2-diacyl-sn-glycero-3-phosphoethanolamines or 1-acyl-2-acyl-sn-glycero-3-phosphoethanolamine, depending on whether they are symmetric or asymmetric lipids. Exemplary phosphatidic acids include dimyristoyl phosphatidic acid, dipalmitoyl phosphatidic acid and dioleoyl phosphatidic acid. Phosphatidic acids may also be referred to under IUPAC naming systems as 1,2-diacyl-sn-glycero-3-phosphate or 1-acyl-2-acyl-sn-glycero-3-phosphate, depending on whether they are symmetric or asymmetric lipids. Exemplary phosphatidylserines include dimyristoyl phosphatidylserine, dipalmitoyl phosphatidylserine, dioleoylphosphatidylserine, distearoyl phosphatidylserine, palmitoyl-oleylphosphatidylserine and brain phosphatidylserine. Phosphatidylserines may also be referred to under IUPAC naming systems as 1,2-diacyl-sn-glycero-3-[phospho-L-serine] or 1-acyl-2-acyl-sn-glycero-3-[phospho-L-serine], depending on whether they are symmetric or asymmetric lipids. As used herein, the abbreviation “PS” refers to phosphatidylserine. Exemplary phosphatidylglycerols include dilauryloylphosphatidylglycerol, dipalmitoylphosphatidylglycerol, distearoylphosphatidylglycerol, dioleoyl-phosphatidylglycerol, dimyristoylphosphatidylglycerol, palmitoyl-oleoyl-phosphatidylglycerol and egg phosphatidylglycerol. Phosphatidylglycerols may also be referred to under IUPAC naming systems as 1,2-diacyl-sn-glycero-3-[phospho-rac-(1-glycerol)] or 1-acyl-2-acyl-sn-glycero-3-[phospho-rac-(1-glycerol)], depending on whether they are symmetric or asymmetric lipids. The phosphatidylglycerol 1,2-dimyristoyl-sn-glycero-3-[phospho-rac-(1-glycerol)] is abbreviated herein as “DMPG”. The phosphatidylglycerol 1,2-dipalmitoyl-sn-glycero-3-(phospho-rac-1-glycerol) (sodium salt) is abbreviated herein as “DPPG”. Suitable sphingomyelins might include brain sphingomyelin, egg sphingomyelin, dipalmitoyl sphingomyelin, and distearoyl sphingomyelin. Other suitable lipids include glycolipids, sphingolipids, ether lipids, glycolipids such as the cerebrosides and gangliosides, and sterols, such as cholesterol or ergosterol.

In some cases, a lipid structure can comprise cholesterol or a derivative thereof, a phospholipid, a mixture of a phospholipid and cholesterol or a derivative thereof, or a combination. Examples of cholesterol derivatives include, but are not limited to, cholestanol, cholestanone, cholestenone, coprostanol, cholesteryl-2′-hydroxyethyl ether, cholesteryl-4′-hydroxybutyl ether, and mixtures thereof. When a lipid structure comprises a mixture of a phospholipid and cholesterol or a cholesterol derivative, the lipid structure may comprise up to about 40, 50, or 60 mol % of the total lipid present in the lipid structure. One or more phospholipids and/or cholesterol may comprise from about 10 mol % to about 60 mol %, from about 15 mol % to about 60 mol %, from about 20 mol % to about 60 mol %, from about 25 mol % to about 60 mol %, from about 30 mol % to about 60 mol %, from about 10 mol % to about 55 mol %, from about 15 mol % to about 55 mol %, from about 20 mol % to about 55 mol %, from about 25 mol % to about 55 mol %, from about 30 mol % to about 55 mol %, from about 13 mol % to about 50 mol %, from about 15 mol % to about 50 mol % or from about 20 mol % to about 50 mol % of the total lipid present in the lipid structure.

In some embodiments, a delivery vehicle herein is designed to be internalized in an epithelial cell, such as an epithelial cell within the gastrointestinal tract. Peptides, in particular, cell penetrating peptides (CPPs) and cell penetrating peptides having mucus-penetrating functionality (MPPs) provide for internalization into the cell. The delivery vehicles herein, such as the lipid structures described herein for such purposes further comprise mucus-penetrating peptide (MPPs), cell-penetrating peptides (CPP), or both. In some embodiments, cell penetrating peptides (CPPs) can be short polypeptides that can allow for increased uptake of delivery vehicles and/or cargo into cells. Cell-penetrating peptides (CPPs) can be peptide sequences that facilitate crossing the cytoplasmic membrane efficiently. Exemplary CPPs and MPPs include those disclosed in PCT/US17/61111 and PCT/US2019/032484 herein incorporated by reference.

In some embodiments, mucus-penetrating cell-penetrating peptides (MPPs) are utilized in conjunction with delivery vehicles described herein. MPPs have cell-penetrating properties and in addition, permit penetration through a layer of mucus such as the naturally-occurring layers of mucus in the colon, lung, eye and cervix. MPP can further be used to target structures to intracellular components of cells. They can also be designed to specifically target certain cell types. MPPs can be conjugated to delivery vehicles to allow penetration of the particles through the mucus layer and also for interaction with cells so as to result in increased penetration or targeting of cells. In some embodiments, a lipid structure that has an MPP can be internalized into a cell with an efficacy of at least about 20%, 25%, 30%, 35%, 40%, 45%, 50%, 55%, 60%, 65%, 70%, 75%, 80%, 85%, 90%, 95%, or up to about 100% as compared to a comparable particles that does not contain an MPP. In some embodiments, a delivery vehicle can comprise a mucus-penetrating peptide (MPP). The MPP may be conjugated to the lipid structure, such as conjugated to a lipid nanoparticle, a surface modification of the lipid nanoparticle or the cargo, such that the MPP is exposed such that it may come into contact, in whole or in part, with a mucus layer, mucus-containing tissue, organ or extracellular surface. The presence of the MPP can confer improved penetration of the delivery vehicle through the mucus (diffusion and/or movement through). In some embodiments, the penetration may be improved 2-fold, 3-fold, 4-fold, 5-fold, 6-fold, 7-fold, 8-fold, 9-fold, 10-fold, 15-fold, 20-fold, 25-fold, 30-fold, 50-fold, 100-fold, or more as compared to the delivery of the delivery vehicle and/or cargo that does not the MPP. In some embodiments, an MPP can have an amino acid sequence having from about 3 to 100 amino acids, including without limitation from about 3 to 5, 5 to 10, 10 to 20, 20 to 40, 30 to 60, or 80 to 100 amino acids. An MPP can have from about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or up to about 100 amino acids. In some embodiments, an MPP may have the ability to penetrate a mucus-layer that overlays or surrounds a target cell or tissue. An MPP can be employed to penetrate the mucus layer of a target tissue such as the intestinal epithelium, colon, lung, eye or cervix of a mammal. MPPs can be conjugated to delivery vehicles, including nanoparticles, to allow penetration of the delivery vehicle through the mucus layer and also for interaction with cells so as to result in increased penetration or targeting of cells. In some embodiments, a particle that has an MPP permeates a mucus layer with an efficacy of at least about 20%, 25%, 30%, 35%, 40%, 45%, 50%, 55%, 60%, 65%, 70%, 75%, 80%, 85%, 90%, 95%, or up to about 100% as compared to a comparable particles that does not contain an MPP. Numerous methods of determining the penetration of a mucus layer can be used to assess the penetration by an MPP or an MPP conjugated directly or indirectly with a delivery vehicle.

In an aspect, a lipid structure can be a mucus-penetrating particle or MPP as used herein, can refer to particles which have been coated with a mucosal penetration enhancing coating. In some cases, a particle can be or can deliver a particle of an active agent, such as a therapeutic, diagnostic, prophylactic, and/or nutraceutical agent (i.e., drug particle) that can be coated with a mucosal penetrating enhancing coating. In other cases, particles can be formed of a matrix material, such as a polymeric material, in which a therapeutic, diagnostic, prophylactic, and/or nutraceutical agent can be encapsulated, dispersed, and/or associated.

In certain cases, a delivery vehicle can further comprise at least one targeting agent. The term targeting agent can refer to a moiety, compound, antibody, etc. that specifically binds a particular type or category of cell and/or other particular type compounds, (e.g., a moiety that targets a specific cell or type of cell). A targeting agent can be specific (e.g., have an affinity) for the surface of certain target cells, a target cell surface antigen, a target cell receptor, or a combination thereof. In some cases, a targeting agent can refer to an agent that has a particular action (e.g., cleaves) when exposed to a particular type or category of substances and/or cells, and this action can drive the delivery vehicle to target a particular type or category of cell. Thus, the term targeting agent can refer to an agent that can be part of a delivery vehicle and plays a role in the delivery vehicle's targeting mechanism, although the agent itself may or may not be specific for the particular type or category of cell itself. In certain instances, the efficiency of the cellular uptake of a polynucleic acid delivered by a delivery vehicle can be enhanced and/or made more specific by incorporation of targeting agents into the present delivery vehicles. In certain embodiments, delivery vehicles described herein can comprise one or more small molecule targeting agents (e.g., carbohydrate moieties). Suitable targeting agents also include, by way of non-limiting example, antibodies, antibody-like molecules, or peptides, such as an integrin-binding peptides such as RGD-containing peptides, or small molecules, such as vitamins, e.g., folate, sugars such as lactose and galactose, or other small molecules. Cell surface antigens include a cell surface molecule such as a protein, sugar, lipid or other antigen on the cell surface. In specific embodiments, the cell surface antigen undergoes internalization. Examples of cell surface antigens targeted by the targeting agents of embodiments of the present delivery vehicles include, but are not limited, to the transferrin receptor type 1 and 2, the EGF receptor, HER2/Neu, VEGF receptors, integrins, NGF, CD2, CD3, CD4, CDS, CDI9, CD20, CD22, CD33, CD43). CD56, CD69, and the leucine-rich repeat-containing G-protein coupled receptor 5 (LGR5). A targeting agent can also comprise an artificial affinity molecule, e.g., a peptidomimetic or an aptamer. Peptidomimetics can refer to compounds in which at least a portion of a peptide, such as a therapeutic peptide, is modified, and the three-dimensional structure of the peptidomimetic remains substantially the same as that of the peptide. Peptidomimetics (both peptide and non-peptidyl analogues) may have improved properties (e.g., decreased proteolysis, increased retention or increased bioavailability). Peptidomimetics generally have improved oral availability, which makes them especially suited to treatment of disorders in a human or animal. It should be noted that peptidomimetics may or may not have similar two-dimensional chemical structures but share common three-dimensional structural features and geometry.

In some embodiments, the targeting agent can be a proteinaceous targeting agent (e.g., a peptide, and antibody, an antibody fragment). In some specific embodiments, a delivery vehicle can comprise a plurality of different targeting agents. In embodiments, a lipid structure modification can provide biocompatibility and can be modified to possess targeting species including, for example, targeting peptides including antibodies, aptamers, polyethylene, or combinations thereof. A targeting agent be a receptor. In some cases, a T cell receptor (TCR), B cell receptor (BCR), single chain variable fragment (scFv), chimeric antigen receptor (CAR), or combinations thereof are used as targeting agents.

In some embodiments, one or more targeting agents can be coupled to the polymers that form the delivery vehicle. In some cases, the targeting agents can be bound to a polymer that coats a delivery vehicle. In some instances, a targeting agent can be covalently coupled to a polymer. In some cases, a targeting agent can be bound to a polymer such that a targeting agent can be substantially at or near the surface of the resulting delivery vehicle. In certain embodiments, a monomer comprising a targeting agent residue (e.g., a polymerizable derivative of a targeting agent such as an (alkyl) acrylic acid derivative of a peptide) can be co-polymerized to form the copolymer forming the delivery vehicle provided herein. In certain embodiments, one or more targeting agents can be coupled to the polymer of the present delivery vehicles through a linking moiety. In some embodiments, the linking moiety coupling the targeting agent to the membrane-destabilizing polymer can be a cleavable linking moiety (e.g., comprises a cleavable bond). In some embodiments, the linking moiety can be cleavable and/or comprises a bond that can be cleavable in endosomal conditions. In some embodiments, the linking moiety can be cleavable and/or comprise a bond that can be cleaved by a specific enzyme (e.g., a phosphatase, or a protease). In some embodiments, the linking moiety can be cleavable and/or comprise a bond that may be cleavable upon a change in an intracellular parameter (e.g., pH, redox potential), in some embodiments, a linking moiety can be cleavable and/or comprise a bond that can be cleaved upon exposure to a matrix metalloproteinase (MMP) (e.g., MMP-cleavable peptide linking moiety).

In certain cases, a targeting mechanism of a delivery vehicle can depend on a cleavage of a cleavable segment in a polymer. For instance, the present polymers can comprise a cleavable segment that, when cleaved, exposes the delivery vehicle and/or the core of a delivery vehicle. The cleavable segment can be located at either or both terminal ends of the present polymers in some embodiments. In some embodiments the cleavable segment is located along a length of a polymer, and optionally can be located between blocks of a polymer. For example, in certain embodiments the cleavable segment can be located between a first block and a second block of a polymer, and when a delivery vehicle can be exposed to a particular cleaving substance the first block can be cleaved from a second block. In specific embodiments a cleavable segment can be an MMP-cleavable peptide that can be cleaved upon exposure to MMP.

Attachment of a targeting agent, such as an antibody or a peptide, to a polymer or a lipid can be achieved in any suitable manner, e.g., by any one of a number of conjugation chemistry approaches including but not limited to amine-carboxyl linkers, amine-sulfhydryl linkers, amine-carbohydrate linkers, amine-hydroxyl linkers, amine-amine linkers, carboxyl-sulfhydryl linkers, carboxyl-carbohydrate linkers, carboxyl-hydroxyl linkers, carboxyl-carboxyl linkers, sulfhydryl-carbohydrate linkers, sulfhydryl-hydroxyl tinkers, sulfhydryl-sulfhydryl linkers, carbohydrate-hydroxyl linkers, carbohydrate-carbohydrate linkers, and hydroxyl-hydroxyl linkers. In specific embodiments, “click” chemistry can be used to attach the targeting agent to the polymers of the delivery vehicles provided herein. A large variety of conjugation chemistries are optionally utilized, in some embodiments, targeting agents can be attached to a monomer and the resulting compound can then be used in a polymerization synthesis of a polymer (e.g., copolymer) utilized in a delivery vehicle described herein. In some embodiments, a targeting agent can be attached to the sense or antisense strand of siRNA bound to a polymer of a delivery vehicle. In certain embodiments, a targeting agent can be attached to a 5′ or a 3′ end of the sense or the antisense strand.

Methods for linking compounds can include but are not limited to proteins, labels, and other chemical entities, to nucleotides. Cross-linking reagents such as n-maleimidobutyryloxy-succinimide ester (GMBS) and sulfo-GMBS, have reduced immunogenicity. Substituents have been attached to the 5′ end of preconstructed oligonucleotides using amidite or H-phosphonate chemistry. Substituents can also be attached to the 3′ end of oligomers. This last method utilizes 2,2′-dithioethanol attached to a solid support to displace diisopropylamine from a 3′ phosphonate bearing the acridine moiety and is subsequently deleted after oxidation of the phosphorus. Alternatively, an oligonucleotide may include one or more modified nucleotides having a group attached via a linker arm to the base. For example, the attachment of biotin to the C-5 position of dUTP by an allylamine linker arm may be utilized. The attachment of biotin and other groups to the 5-position of pyrimidines via a linker arm may also be performed.

Chemical cross-linking may include the use of spacer arms, i.e., linkers or tethers. Spacer arms provide intramolecular flexibility or adjust intramolecular distances between conjugated moieties and thereby may help preserve biological activity. A spacer arm may be in the form of a peptide moiety comprising spacer amino acids. Alternatively, a spacer arm may be part of the cross-linking reagent, such as in “long-chain SPDP”.

A variety of coupling or crosslinking agents such as protein A, carbodiimide, dimaleimide, dithio-bis-nitrobenzoic acid (DTNB), N-succinimidyl-5-acetyl-thioacetate (SATA), and N-succinimidyl-3-(2-pyrid-yldithio)propionate (SPDP), 6-hydrazinonicotimide (HYNIC), N₃S and N₂S₂ can be used in well-known procedures to synthesize targeted constructs. For example, biotin can be conjugated to an oligonucleotide via DTPA using a bicyclic anhydride method. In addition, sulfosuccinimidyl 6-(biotinamido)hexanoate (NHS-LC-biotin, which can be purchased from Pierce Chemical Co. Rockford, Ill.), “biocytin,” a lysine conjugate of biotin, can be useful for making biotin compounds due to the availability of a primary amine. In addition, corresponding biotin acid chloride or acid precursors can be coupled with an amino derivative of the therapeutic agent by known methods. By coupling a biotin moiety to the surface of a particle, another moiety may be coupled to avidin and then coupled to the particle by the strong avidin-biotin affinity, or vice versa. In certain embodiments where a polymeric particle comprises PEG moieties on the surface of the particle, the free hydroxyl group of PEG may be used for linkage or attachment (e.g., covalent attachment) of additional molecules or moieties to the particle.

In an aspect, the lipid structures herein (delivery vehicles) have sizes can fall into the nanometer to micrometer range, such as 20-200 nm, 200 nm-1 m. In some cases, a polynucleic acid may be condensed to be properly encapsulated by a lipid structures. Condensation of DNA may be performed by divalent metal ions such as Mn²⁺, Ni²⁺, Co²⁺, and Cu²⁺ that can condense DNA through neutralization of phosphate groups of the DNA backbone and distortion of the B-DNA structure through hydrogen bonding with bases, permitting both local bending of the DNA and inter-helical associations. In some cases, the concentration of metal ions utilized for condensation can be dependent on the dielectric constant of a medium used in the condensation. The addition of ethanol or methanol may also reduce the concentration of metal ion required for condensation. In some cases, ethanol can be used to condense DNA at concentrations from about 0.5% up to about 60% by volume. In some cases, ethanol can be used to condense DNA at concentrations from about 0.5%, 5%, 10%, 15%, 20%, 25%, 30%, 35%, 40%, 45%, 50%, 55% or up to 60% by volume. In some cases, calcium may also be used for condensation. Calcium not only binds to DNA phosphates but can also form a complex with the nitrogen and oxygen of guanine, disrupting base pairing.

In some cases, a polynucleic acid can be fully encapsulated in a lipid structures. Full encapsulation can indicate that a polynucleic acid in a lipid structures may not be significantly degraded after exposure to serum or a nuclease or protease assay that would significantly degrade free DNA, RNA, or protein. In a fully encapsulated system, preferably less than about 25% of a polynucleic acid in a lipid structure can be degraded in a treatment that would normally degrade 100% of free polynucleic acid, more preferably less than about 10%, and most preferably less than about 5% of a polynucleic acid in a lipid structure can be degraded. In the context of polynucleic acids, full encapsulation may be determined by an Oligreen® assay. Oligreen® is an ultra-sensitive fluorescent nucleic acid stain for quantitating oligonucleotides and single-stranded DNA or RNA in solution (available from Invitrogen Corporation; Carlsbad, Calif.). “Fully encapsulated” can also indicate that a lipid structure may be serum-stable, that is, that they do not rapidly decompose into their component parts upon in vivo administration.

In certain applications, it may be desirable to release a moiety once a drug such as a polynucleic acid has entered a cell. A moiety can be utilized to identify a number of cells that have received a polynucleic acid. A moiety can be an antibody, dye, scFv, peptide, glycoprotein, carbohydrate, ligand, polymer, to name a few. A moiety can be in contact with a linker. A linker can be non-cleavable. Accordingly, in some cases, a linker can be a cleavable linker. This may enable a moiety to be released from a lipid structure once contact to a target cell has been made. This may be desirable when a moiety has a greater therapeutic effect when separated from a lipid structure. In some cases, a moiety may have a better ability to be absorbed by an intracellular component of a cell, such as an intestinal crypt cell or intestinal crypt stem cell, when separated from a lipid structure. In some cases, a linker may comprise a disulfide bond, acyl hydrazone, vinyl ether, orthoester, or a N—PO3.

Accordingly, it may be necessary or desirable to separate a moiety from a lipid structure so that a moiety can enter an intracellular compartment. Cleavage of a linker releasing a moiety may be as a result of a change in conditions within a cell as compared to outside cells, for example, due to a change in pH within a cell. Cleavage of a linker may occur due to the presence of an enzyme within a cell which cleaves a linker once a drug, such as a polynucleic acid, enters a cell. Alternatively, cleavage of a linker may occur in response to energy or a chemical being applied to the cell. Examples of types of energies that may be used to effect cleavage of a linker include, but are not limited to light, ultrasound, microwave and radiofrequency energy. In some cases, a linker may be a photolabile linker. A linker used to link a complex may also be an acid labile linker. Examples of acid labile linkers include linkers formed by using cis-aconitic acid, cis-carboxylic alkatriene, polymaleic anhydride, and other acid labile linkers.

In some cases, a lipid structure, such as a liposome can be biocompatible and biodegradable. For example, in some cases, a liposome may biodegrade after introduction into a subject. Biodegradation can begin immediately after introduction in some cases. Biodegradation can occur within a mucosal tract of a subject that has received an administration of a liposome or liposomal structure. Biodegradation can result release of a liposomal cargo such as a polynucleic acid. In other cases, biodegradation can comprise decomposition of a component of a liposomal structure such as a polymer. Biodegradation can occur under standard bodily conditions such as from about 97.6° F. to about 99° F. In other cases, biodegradation can occur under a temperature from about 95° F. to about 106° F. Biodegradation can occur from about 95° F., 96° F., 97° F., 98° F., 99° F., 100° F., 101° F., 102° F., 103° F., 104° F., 105° F., or up to 106° F. In other aspects, biodegradation can occur from about 50° F. to about 150° F.

In other cases, biodegradation may not occur. When biodegradation occurs, it can take from about 1 minute to about 100 years after administration of a liposome or a structure to a subject. Biodegradation can take from about 1 minute, 5 minutes, 30 minutes, 1 hour, 3 hours, 7 hours, 10 hours, 15 hours, 20 hours, 25 hours, 2 days, 4 days, 8 days, 12 days, 20 days, 30 days, 1.5 months, 3 months, 4 months, 5 months, 6 months, 7 months, 8 months, 9 months, 10 months, 11 months, 12 months, 1.5 yrs., 3 years, 5 years, 8 years, 10 years, 15 years, 20 years, 30 years, 40 years, 50 years, 60 years, 70 years, 80 years, 90 years, or at least about 100 years. Lipid of a structure such as a liposome may be or may comprise: fatty acids, glycerolipids, glycerophospholipids, sphingolipids, saccharolipids, polyketides (derived from condensation of ketoacyl subunits); sterol lipids prenol lipids (derived from condensation of isoprene subunits) or any combination thereof.

### Cargo

The delivery vehicles herein with charge separation and epithelial-reaching functionality as provided herein can be utilized to deliver any type of cargo to a target, for instance a target cell. In some cases, a cargo can comprise a therapeutic agent. Exemplary therapeutic agents can comprise: a nucleic acid, a protein, an antibody, a peptide, a small molecule, a biologic, an antisense oligonucleotide, peptidomimetics, ribozymes, a chemical agent such as a chemotherapeutic molecule, or any large molecule including, but not limited to, viral particles, growth factors cytokines, immunomodulating agents, small molecule drugs, fluorescent dyes, including fluorescent dye peptides which may be expressed by a DNA incorporated within a liposome, or any combination thereof.

In an aspect, a cargo can be a nucleic acid. A nucleic acid can be DNA- or RNA-based. A nucleic acid can be a vector. DNA-based vectors can be non-viral, and can include molecules such as plasmids, minicircles, nanoplasmid, closed linear DNA (doggybone), linear DNA, and single-stranded DNA. A nucleic acid that can be present in a lipid-nucleic acid particle includes any form of nucleic acid that is known. The nucleic acids used herein can be single-stranded DNA or RNA, or double-stranded DNA or RNA, or DNA-RNA hybrids. Examples of double-stranded DNA include structural genes, genes including control and termination regions, and self-replicating systems such as viral or plasmid DNA. Examples of double-stranded RNA include siRNA and other RNA interference reagents. Single-stranded nucleic acids include antisense oligonucleotides, ribozymes, microRNA, and triplex-forming oligonucleotides. The nucleic acid that is present in a lipid-nucleic acid particle may include one or more of the oligonucleotide modifications described below. Nucleic acids may be of various lengths, generally dependent upon the particular form of nucleic acid. For example, in particular embodiments, plasmids or genes may be from about 1,000 to 100,000 nucleotide residues in length. In particular embodiments, oligonucleotides may range from about 10 to 100 nucleotides in length. In various related embodiments, oligonucleotides, single-stranded, double-stranded, and triple-stranded, may range in length from about 10 to about 50 nucleotides, from about 20 to about 50 nucleotides, from about 15 to about 30 nucleotides, from about 20 to about 30 nucleotides in length. In particular embodiments, oligonucleotides may range from about 2 nucleotides to 10 nucleotides in length.

DNA-based vectors can also be viral, and include adeno-associated virus, lentivirus, adenovirus, and others. Vectors can also be RNA. RNA vectors can be linear or circular forms of unmodified RNA. They can also include various nucleotide modifications designed to increase half-life, decrease immunogenicity, and/or increase level of translation. A vector as used herein can be composed of either DNA or RNA. In some embodiments, a vector can be composed of DNA. Vectors can be capable of autonomous replication in a prokaryote such as E. coli, used for growth. In some embodiments a vector may be stably integrated into a genome of an organism. In other cases, a vector can remain separate, either in a cytoplasm or a nucleus. In some embodiments, a vector can contain a targeting sequence. In some embodiments, a vector can contain an antibiotic resistance gene. A vector can contain regulatory elements for regulating gene expression. In some cases, a mini-circle can be enclosed within a delivery vehicle.

In an aspect, Minicircle (MC) DNA can be delivered as cargo by a vehicle provided herein. MC can be similar to plasmid DNA as both may contain expression cassettes that may permit transgene products to be made at high levels shortly after delivery. In some cases, a MC can differ in that MC DNA can be devoid of prokaryotic sequence elements (e.g., bacterial origin of replication and antibiotic-resistance genes). Removal of prokaryotic sequence elements from a backbone plasmid DNA can be achieved via site-specific recombination in Escherichia coli before episomal DNA isolation. The lack of prokaryotic sequence elements may reduce MC size relative to its parental full-length (FL) plasmid DNA, which may lead to enhanced transfection efficiencies. The result may be that when compared with their FL plasmid DNA counterparts, MCs can transfect more cells and may permit sustained high-level transgene expression upon delivery. In some cases, a minicircle DNA can be free of a bacterial origin of replication. For example, a minicircle DNA or closed linear DNA, can be free of a bacterial origin of replication from about 50% of a bacterial origin of replication sequence or up to 100% of a bacterial origin of replication. In some cases, a bacterial origin of replication is truncated or inactive. A polynucleic acid can be derived from a vector that initially encoded a bacterial origin of replication. A method can be utilized to remove the entirety of a bacterial origin of replication or a portion thereof, leaving a polynucleic acid free of a bacterial origin of replication. In some cases, a bacterial origin of replication can be identified by its high adenine and thymine content. Minicircle DNA vectors can be supercoiled minimal expression cassettes, derived from conventional plasmid DNA by site-specific recombination in vivo in Escherichia coli for the use in non-viral gene therapy and vaccination. Minicircle DNA may lack or have reduced bacterial backbone sequences such as an antibiotic resistance gene, an origin of replication, and/or inflammatory sequences intrinsic to bacterial DNA. In addition to their improved safety profile, minicircles can greatly increase efficiency of transgene expression.

In some cases, a portion of a gene can be delivered by a polynucleic acid cargo. A portion of a gene can be from three nucleotides up to the entire whole genomic sequence. For example, a portion of a gene can be from about 1% up to about 100% of an endogenous genomic sequence. A portion of a gene can be from about 1%, 10%, 20%, 30%, 40%, 50%, 60%, 70%, 80%, 90%, or up to about 100% of a whole genomic sequence of a gene.

A variety of protein and polypeptides can be delivered as cargo by vehicles described herein including but not limited to proteins for treating metabolic disorders and endocrine disorders. Examples of proteins are phenylalanine hydroxylase, insulin, anti-diuretic hormone and growth hormone. Disorders include phenylketonuria, diabetes, organic acidurias, tyrosinemia, urea cycle disorders, familial hypercholesteremia. Genes for any of the proteins or peptides which can correct the defects in phenylketonuria, diabetes, organic acidurias, tyrosinemia, urea cycle disorders, familial hypercholesteremia can be introduced into stem cells such that the protein or peptide products are expressed by the intestinal epithelium. Coagulation factors such as antihemophilic factor (factor 8), Christmas factor (factor 9) and factor 7 can likewise be produced in the intestinal epithelium. Proteins which can be used to treat deficiency of a circulatory protein can also be expressed in the intestinal epithelium. Proteins which can be used to treat deficiency of a circulatory protein can be, for example, albumin for the treatment of an albuminemia, alpha-1-antitrypsin, hormone binding protein. Additionally, the intestinal symptoms of cystic fibrosis can be treated by inserting the gene for the normal cystic fibrosis transmembrane conductance regulator into the stem cells of intestinal epithelium. Abetalipoproteinemia can be treated by the insertion of the apolipoprotein B. Disaccharidase intolerance can be treated by the insertion of sucrase-isomaltose, lactase-phlorizin hydrolase and maltase-glucoamylase. The insertion of the intrinsic factor for the absorption of vitamin B₁₂ or the receptor for the intrinsic factor/cobalamin complex for absorption of vitamin B₁₂, as well as the transporter for bile acids can be inserted into the intestinal epithelium. Further, any drug which can be encoded by nucleic acid can be inserted into the stem cell of the intestinal epithelium to be secreted in localized, high concentrations for the treatment of cancer. In this respect, one skilled in the art will readily recognize that antisense RNA can be encoded into the stem cells after production of antisense it can incorporate into the cancerous cells for the treatment of cancer.

A therapeutic agent or drug can be a small molecule, protein, polysaccharide or saccharide, nucleic acid molecule, lipid, peptidomimetic, or a combination thereof. A delivery vehicle can include any molecule or compound capable of exerting a desired effect on a cell, tissue, organ, or subject. Such effects may be biological, physiological, or cosmetic, for example. Molecules or compounds may include e.g., nucleic acids, peptides and polypeptides, including, e.g., antibodies, such as, e.g., polyclonal antibodies, monoclonal antibodies, antibody fragments; humanized antibodies, recombinant antibodies, recombinant human antibodies, and Primatized™ antibodies, cytokines, growth factors, apoptotic factors, differentiation-inducing factors, cell surface receptors and their ligands; hormones; and small molecules, including small organic molecules or compounds. In one embodiment, a molecules or compound can be a therapeutic agent, or a salt or derivative thereof. Therapeutic agent derivatives may be therapeutically active themselves or they may be prodrugs, which become active upon further modification. Thus, in one embodiment, a molecules or compound derivative may retain some or all of the therapeutic activity as compared to the unmodified agent, while in another embodiment, a therapeutic derivative lacks therapeutic activity.

In various embodiments, therapeutic agents include any therapeutically effective agent or drug, such as anti-inflammatory compounds, anti-depressants, stimulants, analgesics, antibiotics, birth control medication, antipyretics, vasodilators, anti-angiogenics, cytovascular agents, signal transduction inhibitors, cardiovascular drugs, e.g., anti-arrhythmic agents, vasoconstrictors, hormones, and steroids. In certain embodiments, a molecule or compound can be an oncology drug, which may also be referred to as an anti-tumor drug, an anti-cancer drug, a tumor drug, an antineoplastic agent, or the like. Examples of oncology drugs that may be used include, but are not limited to, adriamycin, alkeran, allopurinal, altretamine, amifostine, anastrozole, araC, arsenic trioxide, azathioprine, bexarotene, biCNU, bleomycin, busulfan intravenous, busulfan oral, capecitabine (Xeloda), carboplatin, carmustine, CCNU, celecoxib, chlorambucil, cisplatin, cladribine, cyclosporin A, cytarabine, cytosine arabinoside, daunorubicin, cytoxan, daunorubicin, dexamethasone, dexrazoxane, dodetaxel, doxorubicin, doxorubicin, DTIC, epirubicin, estramustine, etoposide phosphate, etoposide and VP-16, exemestane, FK506, fludarabine, fluorouracil, 5-FU, gemcitabine (Gemzar), gemtuzumab-ozogamicin, goserelin acetate, hydrea, hydroxyurea, idarubicin, ifosfamide, imatinib mesylate, interferon, irinotecan (Camptostar, CPT-111), letrozole, leucovorin, leustatin, leuprolide, levamisole, litretinoin, megastrol, melphalan, L-PAM, mesna, methotrexate, methoxsalen, mithramycin, mitomycin, mitoxantrone, nitrogen mustard, paclitaxel, pamidronate, Pegademase, pentostatin, porfimer sodium, prednisone, rituxan, streptozocin, STI-571, tamoxifen, taxotere, temozolamide, teniposide, VM-26, topotecan (Hycarntin), toremifene, tretinoin, ATRA, valrubicin, velban, vinblastine, vincristine, VP16, and vinorelbine. Other examples of oncology drugs that may be used are ellipticin and ellipticin analogs or derivatives, epothilones, intracellular kinase inhibitors and camptothecins.

In some aspects, a polynucleic acid for use as a cargo with the delivery vehicles herein include nucleic acids encoding for a tumor-suppressor gene. A tumor-suppressor gene can generally encode for a protein that in one way or another can inhibit cell proliferation. Loss of one or more of these “brakes” may contribute to the development of a cancer. Five broad classes of proteins can be generally recognized as being encoded by tumor-suppressor genes: Intracellular proteins, such as the p16 cyclin-kinase inhibitor, that can regulate or inhibit progression through a specific stage of the cell cycle, receptors for secreted hormones (e.g., tumor derived growth factor j) that may function to inhibit cell proliferation, checkpoint-control proteins that arrest the cell cycle if DNA may be damaged or chromosomes are abnormal, proteins that can promote apoptosis, enzymes that participate in DNA repair, or a combination thereof. Although DNA-repair enzymes may not directly function to inhibit cell proliferation, cells that have lost the ability to repair errors, gaps, or broken ends in DNA accumulate mutations in many genes, including those that are critical in controlling cell growth and proliferation. Thus loss-of-function mutations in the genes encoding DNA-repair enzymes may promote inactivation of other tumor-suppressor genes as well as activation of oncogenes. Since generally one copy of a tumor-suppressor gene suffices to control cell proliferation, both alleles of a tumor-suppressor gene must be lost or inactivated in order to promote tumor development. In an aspect, oncogenic loss-of-function mutations in tumor-suppressor genes act recessively. Tumor-suppressor genes in many cancers have deletions or point mutations that prevent production of any protein or lead to production of a nonfunctional protein. In some cases, introducing a tumor suppressor gene encoding for a protein may ameliorate disease, prevent disease, or treat disease in a subject.

A tumor suppressor gene that can be delivered by a delivery vehicle herein includes, for example, APC, ARHGEF12, ATM, BCL11B, BLM, BMPR1A, BRCA1, BRCA2, CARS, CBFA2T3, CDH1, CDH11, CDK6, CDKN2C, CEBPA, CHEK2, CREB1, CREBBP, CYLD, DDX5, EXT1, EXT2, FBXW7, FH, FLT3, FOXP1, GPC3, IDH1, IL2, JAK2, MAP2K4, MDM4, MEN1, MLH1, MSH2, NF1, NF2, NOTCHI, NPM1, NR4A3, NUP98, PALB2, PML, PTEN, RB1, RUNX1, SDHB, SDHD, SMARCA4, SMARCB1, SOCS1, STK11, SUFU, SUZ12, SYK, TCF3, TNFAIP3, TP53, TSC1, TSC2, VHL, WRN, WT1, and any combination thereof.

In certain embodiments, a vehicle can comprise an imaging agent that may be further attached to a detectable label (e.g., the label can be a radioisotope, fluorescent compound, enzyme or enzyme co-factor). The active moiety may be a radioactive agent, such as: radioactive heavy metals such as iron chelates, radioactive chelates of gadolinium or manganese, positron emitters of oxygen, nitrogen, iron, carbon, or gallium, ⁴³K, ⁵²Fe, ⁵⁷Co, ⁶⁷Cu, ⁶⁷Ga, ⁶⁸Ga, ¹²³I, ¹²⁵I, ¹³¹I, ¹³²I, or ⁹⁹Tc. A delivery vehicle including such a moiety may be used as an imaging agent and be administered in an amount effective for diagnostic use in a mammal such as a human. In this manner, the localization and accumulation of the imaging agent can be detected. The localization and accumulation of the imaging agent may be detected by radioscintiography, nuclear magnetic resonance imaging, computed tomography, or positron emission tomography. As will be evident to the skilled artisan, the amount of radioisotope to be administered is dependent upon the radioisotope. Those having ordinary skill in the art can readily formulate the amount of the imaging agent to be administered based upon the specific activity and energy of a given radionuclide used as the active moiety. Typically, 0.1-100 millicuries per dose of imaging agent, 1-10 millicuries, and 2-5 millicuries can be administered. Thus, compositions useful as imaging agents can comprise a targeting moiety conjugated to a radioactive moiety that can comprise 0.1-100 millicuries, in some embodiments preferably 1-10 millicuries, in some embodiments preferably 2-5 millicuries, in some embodiments more preferably 1-5 millicuries. The means of detection used to detect the label is dependent of the nature of the label used and the nature of the biological sample used, and may also include fluorescence polarization, high performance liquid chromatography, antibody capture, gel electrophoresis, differential precipitation, organic extraction, size exclusion chromatography, fluorescence microscopy, or fluorescence activated cell sorting (FACS) assay. A targeting moiety can also refer to a protein, nucleic acid, nucleic acid analog, carbohydrate, or small molecule. The entity may be, for example, a therapeutic compound such as a small molecule, or a diagnostic entity such as a detectable label. A locale may be a tissue, a particular cell type, or a subcellular compartment. In one embodiment, the targeting moiety can direct the localization of an active entity. The active entity may be a small molecule, protein, polymer, or metal. The active entity, such as a liposome comprising a nucleic acid, may be useful for therapeutic, prophylactic, or diagnostic purposes. In some cases, a moiety may allow a delivery vehicle to penetrate a blood brain barrier.

A cargo can be a drug. A drug can be a substance that when administered can cause a physiological change in a subject. A drug can be a medication used to treat a disease, such as cancer. In some instances, drugs can be entrapped completely in a liposomal lipid bilayer, in an aqueous compartment, or in both a liposomal lipid bilayer and an aqueous compartment. Strongly lipophilic drugs can be entrapped almost completely in a lipid bilayer. Strongly hydrophilic drugs can be located exclusively in an aqueous compartment. Drugs with intermediate log P can easily partition between a lipid and aqueous phases, both in a bilayer and in an aqueous core. Exemplary drugs can comprise drugs such as adalimumab, anti-TNF, insulin-like growth factor, interleukin, Mesalamine, GLP-1 analogs, GLP-2 analogs, and combinations thereof.

In some cases, a polynucleic acid can encode for a heterologous sequence. A heterologous sequence can provide for subcellular localization (e.g., a nuclear localization signal (NLS) for targeting to a nucleus; a mitochondrial localization signal for targeting to a mitochondria; a chloroplast localization signal for targeting to a chloroplast; an ER retention signal; and the like). In some case, a polynucleic acid, such as minicircle DNA or closed linear DNA, can comprise a nuclear localization sequence (NLS).

A cargo can comprise one or more nuclear localization sequences (NLSs). A number of NLS sequences can be from about 1, 2, 3, 4, 5, 6, 7, 8, 9, 10, or more NLSs. In some embodiments, a vector comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g. one or more NLS at the amino-terminus and one or more NLS at the carboxy terminus). When more than one NLS is present, each may be selected independently of the others, such that a single NLS may be present in more than one copy and/or in combination with one or more other NLSs present in one or more copies. Non-limiting examples of NLSs can include an NLS sequence derived from: the NLS of the SV40 virus large T-antigen, having the amino acid sequence PKKKRKV (SEQ ID NO: 1); the NLS from nucleoplasmin (e.g. the nucleoplasmin bipartite NLS with the sequence KRPAATKKAGQAKKKK (SEQ ID NO: 2)); the c-myc NLS having the amino acid sequence PAAKRVKLD (SEQ ID NO: 3) or RQRRNELKRSP (SEQ ID NO: 4); the hRNPA1 M9 NLS having the sequence NQSSNFGPMKGGNFGGRSSGPYGGGGQYFAKPRNQGGY (SEQ ID NO: 5); the sequence RMRIZFKNKGKDTAELRRRRVEVSVELRKAKKDEQILKRRNV (SEQ ID NO: 6) of the IBB domain from importin-alpha; the sequences VSRKRPRP (SEQ ID NO: 7) and PPKKARED (SEQ ID NO: 8) of the myoma T protein; the sequence POPKKKPL (SEQ ID NO: 9) of human p53; the sequence SALIKKKKKMAP (SEQ ID NO: 10) of mouse c-abl IV; the sequences DRLRR (SEQ ID NO: 11) and PKQKKRK (SEQ ID NO: 11) of the influenza virus NS1; the sequence RKLKKKIKKL (SEQ ID NO: 12) of the Hepatitis virus delta antigen; the sequence REKKKFLKRR (SEQ ID NO: 13) of the mouse Mx1 protein; the sequence KRKGDEVDGVDEVAKKKSKK (SEQ ID NO: 14) of the human poly(ADP-ribose) polymerase; and the sequence RKCLQAGMNLEARKTKK (SEQ ID NO: 15) of the steroid hormone receptors (human) glucocorticoid. In general, the one or more NLSs can be of sufficient strength to drive accumulation of the minicircle DNA vector or short linear DNA vector in a detectable amount in the nucleus of a eukaryotic cell. A eukaryotic cell can be a human intestinal crypt cell.

In some cases, a particle may contain a DNAse inhibitor. A DNAse inhibitor may be localized within a particle or on a particle. In other cases, a polynucleic acid encoding for an inhibitor can be enclosed within a particle. In other cases, an inhibitor can be a DNA methyltransferase inhibitor such as DNA methyltransferase inhibitors-2 (DMI-2). DMI-2 can be produced by Streptomyces sp. strain No. 560. A structure of DMI-2 can be 4′″R,6aR,10S,10aS-8-acetyl-6a,10a-dihydroxy-2-methoxy-12-methyl-10-[4′-[3″-hydroxy-3″,5″-dimethyl-4″ (Z-2′″,4′″-dimethyl-2′″-heptenoyloxy) tetrahydropyran-1″-yloxy]-5′-methylcyclohexan-1′-yloxy]-1,4,6,7,9-pentaoxo-1,4,6,6a,7,8,9,10,10a,11-decahydronaphthacene. Other inhibitors, such as chloroquine, can also be enclosed within a particle or on a particle, such as on a surface of a particle.

Detection of accumulation in the nucleus may be performed by any suitable technique. For example, a detectable marker may be fused to a vector, such that location within a cell may be visualized, such as in combination with a means for detecting the location of the nucleus (e.g. a stain specific for the nucleus such as DAPI). Cell nuclei may also be isolated from cells, the contents of which may then be analyzed by any suitable process for detecting protein, such as immunohistochemistry, Western blot, or enzyme activity assay. An embodiment herein can exhibit time dependent pH triggered release of a cargo into a target site. An embodiment herein can contain and provide cellular delivery of complex multiple cargoes. An additional cargo can be a small molecule, an antibody, an inhibitor such as a DNAse inhibitor or RNAse inhibitor.

A lipid structure can carry to a capacity up to over 100% weight: defined as (cargo weight/weight of the lipid structure)×100. The optimal loading of cargo can be or can be from about 1% to 100% weight of a lipid structure. For example, a lipid structure can contain a polynucleic acid cargo from about 1% weight of a structure to about 10%, from about 10% to about 20%, from about 20% to about 30%, from about 30% to about 40%, from about 40% to about 50%, from about 50%, to about 60%, from about 60% to about 70%, from about 70% to about 80%, from about 80% to about 90%, from about 90% to about 100%, from about 100% to about 200%, from about 200% to about 300%, from about 300% to about 400%, from about 400% to about 500% or greater weight of a structure.

Polynucleic acids can be delivered to cells of the intestinal tract. For example, a polynucleic acid can be delivered by the delivery vehicles herein to an intestinal crypt stem cell. For example, a delivered polynucleic acid can be: (1) not normally found in intestinal epithelial stem cells; (2) normally found in intestinal epithelial stem cells, but not expressed at physiological significant levels; (3) normally found in intestinal epithelial stem cells and normally expressed at physiological desired levels in the stem cells or their progeny; (4) any other DNA which can be modified for expression in intestinal epithelial stem cells; and (5) any combination of the above.

In some cases, a protein that is encoded by a polynucleic acid comprised within a lipid structure can be measured and quantified. In some cases, modified cells can be isolated, and a western blot performed on modified cells to determine a presence and a relative amount of protein production as compared to unmodified cells. In other cases, intracellular staining of a protein utilizing flow cytometry can be performed to determine a presence and a relative amount of protein production. Additional assays can also be performed to determine if a protein, such as APC, is functional. For example, modified cells expressing an APC transgene, can be measured for cytosolic 0-catenin expression and compared to unmodified cells. Reduced expression of 0-catenin in the cytosol of modified cells as compared to unmodified cells can be indicative of a functional APC transgene. In other cases, a murine model of FAP can be utilized to determine functionality of a transgene encoding an APC protein. For example, mice with FAP can be treated with modified cells, encoding for APC, and a reduction of FAP disease measured versus untreated mice.

Provided herein can also be additional procedures that can be performed on subjects receiving subject delivery vehicles. Subjects can receive procedures such as blood transfusions, blood draws, computerized tomography scan (CT) can, magnetic resonance imaging (MRI), X rays, radiation therapy, organ transplants, and any combination thereof. In some cases, an evaluation of a lesion, such as a cancerous lesion, can be performed.

In some cases, non-target lesions can be evaluated. A complete response of a non-target lesion can be a disappearance and normalization of tumor marker level. All lymph nodes must be non-pathological in size (less than 10 mm short axis). If tumor markers are initially above the upper normal limit, they must normalize for a patient to be considered a complete clinical response. Non-CR/Non-PD is persistence of one or more non-target lesions and or maintenance of tumor marker level above the normal limit. Progressive disease can be appearance of one or more new lesions and or unequivocal progression of existing non-target lesions. Unequivocal progression should not normally trump target lesion status. In some cases, a best overall response can be the best response recorded from the start of treatment until disease progression/recurrence.

### Delivery of Cargo

Delivery vehicles provided herein can be utilized to deliver cargo to a target cell. In some cases, a target cell is found in a gastrointestinal tract, reproductive tract, circulatory system, respiratory system, musculoskeletal system, excretory system, nervous system, oculatory system, and combinations thereof. In some cases, suitable target cells can be found in any major organ of the body including but not limited to the skin, lungs, heart, liver, stomach, urinary system, reproductive system, intestine, pancreas, kidneys, thymus gland, thyroid, and/or brain. In some cases, a target cell is part of the gastrointestinal tract and is in the anus, rectum, large intestine, small intestine, liver, stomach, esophagus, or mouth. In some cases, a target cell is an enteroendocrine cell, mast cell, enterocyte, brush cell, Paneth cell, or goblet cell. In some cases, a target cell is an enteroendocrine cell and is an EC cell, D cell, CCK cell, L cell, P/D1 cell, or G cell. In some cases, a target cell is in the intestinal epithelium and is selected from an intestinal stem cell, Paneth cell, goblet cell, enterocyte, transit amplifying cell, enteroendocrine cell, or any combination thereof. In some cases, a target cell is an intestinal stem cell. In some cases, a target cell is a crypt cell.

Delivery vehicles can be utilized to introduce cargo to target cells. In some cases, introduction comprises contacting the target cell with the cargo. In other cases, introduction comprises transfecting or transducing the target cell with the cargo. In some cases, the cargo can modify the genome of the cell or exist within the cell extragenomically.

In some embodiments, a delivery vehicle employed may contain a cargo that is delivered to a target cell, for example for expression in the cell and/or to genetically modify a target cell. An efficiency of such delivery, e.g., transfection, with a cargo, such as a polynucleic acid described herein, for example, can be or can be about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9% of the total number of cells that are contacted (in vivo or ex vivo) and/or are present in a tissue or location. An efficiency of such delivery, e.g., transfection, with a cargo, such as a polynucleic acid described herein, for example, can be or can be about 1 fold, 10 fold, 20 fold, 40 fold, 60 fold, 80 fold, 100 fold, 120 fold, 140 fold, 160 fold, 180 fold, 200 fold, 300 fold, 400 fold, 500 fold, or over 1000 fold of the total number of cells that are contacted (in vivo or ex vivo) and/or are present in a tissue or location.

Efficiency of cellular uptake with subject delivery vehicles, such as the compositions described herein (including delivery vehicles with charge separation for epithelial cell reach, with a bile salt for stability in a harsh environment and optionally including other features such as a MPP or other mucus-penetrating feature) can permit efficient penetration and transit (such as through the mucus layer) to the target cells and thereby have an efficient uptake by the target cell(s), for example, uptake can be or can be about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9% of the total number of cells that are contacted. In some embodiments, the compositions can have a higher percent of cellular uptake as compared to a comparable delivery vehicle that does not include a bile salt and/or charge separation or compared to a delivery vehicle which lacks one or more components. The improvement can be from about 10%, 15%, 20%, 25%, 30%, 35%, 40%, 45%, 50%, 55%, 60%, 65%, 70%, or up to about 80% better. In some cases, an efficiency of transfection or delivery of a cargo (such as integration of or expression of protein from a polynucleic acid) delivered to a cell by a delivery vehicle composition as described herein can be from about 5%10%, 15%, 20%, 25%, 30%, 35%, 40%, 45%, 50%, 55%, 60%, or up to 65% better than a comparable delivery vehicle that does not include a bile salt and/or charge separation or compared to a delivery vehicle which lacks one or more components. In some cases, an efficiency of transfection or integration of or expression from a polynucleic acid cargo delivered to a cell by a delivery vehicle composition as described herein can be from about 5%10%, 15%, 20%, 25%, 30%, 35%, 40%, 45%, 50%, 55%, 60%, or up to 65% better than a comparable delivery vehicle that does not include a bile salt and/or charge separation or compared to a delivery vehicle which lacks one or more components.

In some embodiments, the compositions provided herein for delivering a cargo can be functional for at least or at least about 1, 2, 3, 4, 5, 6, 7, 8, 9, 10, 11, 12, 13, 14, 15, 16, 17, 18, 19, 20, 21, 22, 23, 24, 25, 6, 27, 28, 29, 30, 40, 50, 60, 70, 80, 90, or 100 days after introduction to a subject in need thereof. Structures can be functional for at least or at least about 1, 2, 3, 4, 5, 6, 7, 8, 9, 10, 11, or 12 months after introduction into a subject. A delivery vehicle as provided herein, can be functional for at least or at least about 1, 2, 3, 4, 5, 6, 7, 8, 9, 10, 15, 20, 25, or 30 years after introduction to a subject. In some embodiments, a delivery vehicle can be functional for up to the lifetime of a recipient. Further, a delivery vehicle can function at 100% of its normal intended operation. Delivery vehicles can also function 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of their normal intended operation. Function of a delivery vehicle may refer to the efficiency of delivery, persistence of a lipid nanoparticles, stability of a lipid nanoparticles, or any combination thereof.

In some embodiments, the delivery vehicle provided herein can deliver a cargo, such as a nucleic acid to a target cell (such as RNA, DNA (for example, minicircle DNA)). In some cases, function can include a percent of cells that received a nucleic acid from the delivery vehicle composition. In other cases, function can refer to a frequency or efficiency of protein generation from a nucleic acid. For example, a delivery vehicle composition may deliver a nucleic acid to a cell that encodes for at least a portion of a gene, such as APC, and a frequency of efficiency may describe a functionality complete gene as restored or created by the delivery of the cargo.

A nucleic acid cargo concentration in a delivery vehicle composition can be from 0.5 nanograms to 50 micrograms. Such concentration can be from about 0.5 ng, 1 ng, 2 ng, 5 ng, 10 ng, 50 ng, 100 ng, 150 ng, 200 ng, 300 ng, 400 ng, 500 ng, 600 ng, 700 ng, 800 ng, 900 ng, 1000 ng, 1 μg, 2 μg, 5 μg, 10 μg, 20 μg, 30 μg, 40 μg, 50 μg, 60 μg, or up to 50 μg or greater. In some cases, the amount of nucleic acid (e.g., ssDNA, dsDNA, RNA) that may be introduced to a cell by a delivery vehicle may be varied to optimize transfection efficiency and/or cell viability. In some cases, less than about 100 picograms of nucleic acid may be introduced to a subject. In some cases, at least about 100 picograms, at least about 200 picograms, at least about 300 picograms, at least about 400 picograms, at least about 500 picograms, at least about 600 picograms, at least about 700 picograms, at least about 800 picograms, at least about 900 picograms, at least about 1 microgram, at least about 1.5 micrograms, at least about 2 micrograms, at least about 2.5 micrograms, at least about 3 micrograms, at least about 3.5 micrograms, at least about 4 micrograms, at least about 4.5 micrograms, at least about 5 micrograms, at least about 5.5 micrograms, at least about 6 micrograms, at least about 6.5 micrograms, at least about 7 micrograms, at least about 7.5 micrograms, at least about 8 micrograms, at least about 8.5 micrograms, at least about 9 micrograms, at least about 9.5 micrograms, at least about 10 micrograms, at least about 11 micrograms, at least about 12 micrograms, at least about 13 micrograms, at least about 14 micrograms, at least about 15 micrograms, at least about 20 micrograms, at least about 25 micrograms, at least about 30 micrograms, at least about 35 micrograms, at least about 40 micrograms, at least about 45 micrograms, or at least about 50 micrograms, of nucleic acid may be added to each cell sample (e.g., one or more cells being electroporated or otherwise targeted for cargo delivery). In some cases, the amount of nucleic acid (e.g., dsDNA, RNA) required for optimal transfection efficiency and/or cell viability may be specific to the cell type.

In some embodiments, an effective amount of a structure can mean an amount sufficient to increase the expression level of at least one gene which can be decreased in a subject prior to the treatment or an amount sufficient to alleviate one or more symptoms of cancer. For example, an effective amount can be an amount sufficient to increase the expression level of at least one gene selected from the group consisting of gastrointestinal differentiation genes, cell cycle inhibition genes, and tumor suppressor genes by at least 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 1000%, 1500%, or more compared to a reference value or the expression level without the treatment of any compound.

In some embodiments, an effective amount can mean an amount sufficient to decrease the expression level of at least one gene which may be increased in the subject prior to the treatment or an amount sufficient to alleviate one or more symptoms of cancer. For example, an effective amount can be an amount sufficient to decrease the expression level of a gene by at least 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 1000%, 1500%, or more compared to a reference value or the expression level without the treatment of any compound.

In some embodiments, treating comprises reduction of the disease in the subject in need thereof by at least about 1 fold, 5 fold, 10 fold, 20 fold, 40 fold, 80 fold, 100 fold, 300 fold, 600 fold, or 1000 fold as measured by an in vitro or in vivo assay as compared to a comparable subject that does not undergo the administering. In an aspect, reduction of the disease can be the result of an increase or decreases in the expression level of at least one gene in the subject. Various gene expression assays can be utilized and include but are not limited to sequencing, PCR, RT-PCR, western blot, northern blot, ELISA, protein quantification, mRNA quantification, FISH, RNA-Seq, SAGE, or a combination thereof. Additional assays that can be utilized include microscopy, histology, in vivo animal experiments, human experiments, or any combination thereof.

### Methods of Use

The delivery vehicle compositions herein provide delivery to epithelial cells within mucosal tissues, such as those of the gastrointestinal tract (GI tract), as well as find uses in other mucosal tissues such as lung, vagina and eye. The delivery vehicles herein provide penetration through a mucus layer as well as reaching epithelial cells. In some embodiments, a delivery vehicle delivers a cargo to epithelial cells of the GI tract and delivers a cargo (such as those described herein) for purposes of a therapeutic, diagnostic or theranostic purpose.

Exemplary diseases that can be treated with subject delivery vehicles provided herein, particularly such delivery vehicles with a therapeutic cargo, can be cancerous or non-cancerous. Such disease can be cardiovascular disease, a neurodegenerative disease, an ocular disease, a reproductive disease, a gastrointestinal disease, a brain disease, a skin disease, a skeletal disease, a muscoskeletal disease, a pulmonary disease, a thoracic disease, to name a few. A disease can be a genetic disease such as cystic fibrosis, tay-sachs, fragile X, Huntington's, neurofibromatosis, sickle cell, thalassemias, Duchenne's muscular dystrophy, or a combination thereof.

In some aspects, a disease is a gastrointestinal disease. In some cases, a gastrointestinal disease is a monogenic GI disease. In some aspects, a gastrointestinal disease is inherited. In some cases, a gastrointestinal disease is of the epithelium. Suitable gastrointestinal diseases can be: familial adenomatous polyposis (FAP), attenuated FAP, microvillus inclusion disease (MVID), chronic inflammatory bowel disease, chronic inflammatory bowel disease, ileal Crohn's, juvenile polyposis, hereditary diffuse gastric cancer syndrome (HDGC), Peutz-Jeghers syndrome, lynch syndrome, gastric adenocarcinoma and proximal polyposis of the stomach (GAPPS), Li-Fraumeni syndrome, familial gastric cancer, or a combination thereof. A GI disease can produce polyps in a gastrointestinal tract. In some cases, a disease is FAP. FAP can progress to cancer. A gastrointestinal disease can be hereditary. For example, a hereditary gastrointestinal disease can be Gilbert's syndrome, telangiectasia, mucopolysaccaride, Osler-Weber-Rendu syndrome, pancreatitis, keratoacanthoma, biliary atresia, Morquio's syndrome, Hurler's syndrome, Hunter's syndrome, Crigler-Najjar, Rotor's, Peutz-Jeghers' syndrome, Dubin-Johnson, Osteochondroses, Osteochondrodysplasias, polyposis, or a combination thereof.

In some aspects, a subject can be screened for the presence of a disease. Screens can be utilized to identify suitable subjects. In some cases, a disease can be identified by genetic, phenotypic, molecular, or chromosomal screening. In an aspect, a suitable subject is positive for a disease provided herein. For example, a genetic screen can identify a mutation in an APC gene that can result in FAP. In some cases, a screen can comprise analyzing a gene such as CDH1, STK11, SMAD4, MLH1, MSH2, EPCAM, MSH6, PMS2, MYO5B, APC, TP53, portions thereof, promoters thereof, and combinations thereof.

In some cases, the delivery vehicles herein carry a therapeutic cargo (such as a nucleic acid, a protein or a drug) are used to treat a disease affecting the GI tract such as familial polyposis (FAP), attenuated FAP, colorectal cancer, chronic inflammatory bowel disease, ileal Crohn's, Microvillus Inclusion Disease and congenital diarrheas.

In other cases, a gene for delivery by a liposome may be administered to a subject as a preventive measure. For example, a subject may not have diagnosed disease and may appear to be predisposed to a disease such as cancer. In some cases, a cancer can be a colon cancer.

In some cases, the delivery vehicles herein carry a diagnostic cargo and are used to visualize or diagnose the state of cells or tissues or to diagnose or monitor a subject for a condition or a disease. For example, a subject is administered an effective amount of delivery vehicles and a diagnostic method for FAP includes determining a level of APC incorporated into a cell genome whereupon a difference in APC levels before the start of therapy in a patient and during and/or after therapy will evidence the effectiveness of therapy in a patient, including whether a patient has completed therapy or whether the disease state has been inhibited or eliminated.

A pharmaceutical composition containing a delivery vehicle with its cargo can be administered chronically in some cases. Administration can encompass hourly, daily, monthly, or yearly administration of a structure to a subject. For example, in some cases, a subject may be administered a pharmaceutical composition daily for the entirety of the subject's life. In other cases, a pharmaceutical composition may be administered daily for the duration of the presence of disease in a subject. A subject may be administered a pharmaceutical composition, such as with a delivery vehicle and a polynucleic acid cargo, to treat a disease or disorder until the disease or disorder is reduced, controlled, or eliminated. Disease control may encompass the stabilization of a disease. For example, a cancer that is controlled may have stopped growing or spreading as measured by CT scan. A cancer may be a colon cancer. In other cases, a pharmaceutical composition may be administered prophylactically. In some cases, a subject may have undergone a genetic screen that identifies the subject as being predisposed to a cancer, such as colon cancer. In this case, a predisposed subject may begin prophylactic treatment by receiving a pharmaceutical composition comprising delivery vehicle and a polynucleic acid cargo. In the case, that a subject contains a genetic mutation that predisposes the subject to colon cancer, that subject may begin prophylactic treatment with such pharmaceutical composition.

In some cases, prophylactic treatment can prevent a disease, such as cancer. When prevention can be used in relation to a condition, such as a local recurrence (e.g., pain), a disease such as cancer, a syndrome complex such as heart failure or any other medical condition prevention can include administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

Assays can be utilized to determine therapeutic effectiveness of delivery vehicles provided herein. In some cases, an assay can be performed before, during, and/or after administration of subject delivery vehicles. An assay can be performed for example on days −30, −15, −7, −3, 0, 3, 5, 7, 10, 14, 18, 20, 24, 30, 35, 40, 50, 55, 60, 80, 100, 150, 250, 360, 2 years, 5 years, or 10 years pre or post administration. Suitable assays can be in vivo or ex vivo. In some cases, an assay comprises a scan. Suitable scans can comprise CT, PET, MRI, or combinations thereof. In some cases, an assay comprises an in vitro assay such as histology, serology, sequencing, ELISA, microscopy, and the like.

### Pharmaceutical Compositions and Formulations

The compositions described throughout can be formulation into a pharmaceutical medicament and be used to treat a human or mammal, in need thereof. Medicaments can be co-administered with any additional therapy.

For oral administration, an excipient may include pharmaceutical grades of mannitol, lactose, starch, magnesium stearate, sodium saccharine, talcum, cellulose, glucose, gelatin, sucrose, magnesium carbonate, and the like. If desired, a delivery vehicle composition may also contain minor amounts of non-toxic auxiliary substances such as wetting agents, emulsifying agents, or buffers.

A composition can be administered orally, by subcutaneous or other injection, intravenously, intracerebrally, intramuscularly, parenterally, transdermally, nasally or rectally. The form in which the compound or composition is administered depends at least in part on the route by which the compound is administered. In some cases, a composition can be employed in the form of solid preparations for oral administration; preparations may be tablets, granules, powders, capsules or the like. In a tablet formulation, a composition is typically formulated with additives, e.g. an excipient such as a saccharide or cellulose preparation, a binder such as starch paste or methyl cellulose, a filler, a disintegrator, and other additives typically used in the manufacture of medical preparations. A composition to be administered may contain a quantity of a delivery vehicle in a pharmaceutically effective amount for therapeutic use in a biological system, including a patient or subject. A pharmaceutical composition may be administered daily or administered on an as needed basis.

The delivery vehicles herein include those formulated as a pharmaceutical composition for administration. Suitable formulations can include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents. Suitable inert carriers can include sugars such as lactose. In some cases, the compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

A carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride. Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, and combination thereof. Suitable surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimoniuni bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-beta-alanine, sodium N-lauryl-beta-iminodipropionate, myristoamphoacetate, lauryl betaine and lauryl sulfobetaine. The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s). The formulation is typically buffered to a pH of 3-8 for parenteral administration upon reconstitution. Suitable buffers include, but are not limited to, phosphate buffers, acetate buffers, and citrate buffers. Water soluble polymers can be often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions can be prepared by incorporating the various sterilized active ingredients into a sterile vehicle which contains the basic dispersion medium and the required other ingredients from those listed above. In the case of sterile powders for the preparation of sterile injectable solutions, a method of preparation can be vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

A formulation can be an ocular formulation or a topical formation. Pharmaceutical formulations for ocular administration can be in the form of a sterile aqueous solution or suspension of particles formed from one or more polymer-drug conjugates. Acceptable solvents include, for example, water, Ringer's solution, phosphate buffered saline (PBS), and isotonic sodium chloride solution. The formulation may also be a sterile solution, suspension, or emulsion in a nontoxic, parenterally acceptable diluent or solvent such as 1,3-butanediol. In still other embodiments, the liposomes can be formulated for topical administration to mucosa. Suitable dosage forms for topical administration include creams, ointments, salves, sprays, gels, lotions, emulsions, liquids, and transdermal patches. The formulation may be formulated for transmucosal, transepithelial, transendothelial, or transdermal administration. The compositions contain one or more chemical penetration enhancers, membrane permeability agents, membrane transport agents, emollients, surfactants, stabilizers, and combination thereof. In some embodiments, the liposomes can be administered as a liquid formulation, such as a solution or suspension, a semi-solid formulation, such as a lotion or ointment, or a solid formulation. In some embodiments, the liposomes can be formulated as liquids, including solutions and suspensions, such as eye drops or as a semi-solid formulation, such as ointment or lotion for topical application to mucosa, such as the eye or vaginally or rectally. The formulation may contain one or more excipients, such as emollients, surfactants, emulsifiers, and penetration enhancers.

An appropriate dosage (“therapeutically effective amount”) of an active agent(s) in a composition may depend, for example, on the severity and course of a condition, a mode of administration, a bioavailability of a particular agent(s), the age and weight of a subject, a subject's clinical history and response to an active agent(s), discretion of a physician, or any combination thereof. A therapeutically effective amount of an active agent(s) in a composition to be administered to a subject can be in the range of about 100 μg/kg body weight/day to about 1000 mg/kg body weight/day whether by one or more administrations. In some embodiments, the range of each active agent administered daily can be from about 100 g/kg body weight/day to about 50 mg/kg body weight/day, 100 μg/kg body weight/day to about 10 mg/kg body weight/day, 100 μg/kg body weight/day to about 1 mg/kg body weight/day, 100 μg/kg body weight/day to about 10 mg/kg body weight/day, 500 μg/kg body weight/day to about 100 mg/kg body weight/day, 500 μg/kg body weight/day to about 50 mg/kg body weight/day, 500 μg/kg body weight/day to about 5 mg/kg body weight/day, 1 mg/kg body weight/day to about 100 mg/kg body weight/day, 1 mg/kg body weight/day to about 50 mg/kg body weight/day, 1 mg/kg body weight/day to about 10 mg/kg body weight/day, 5 mg/kg body weight/dose to about 100 mg/kg body weight/day, 5 mg/kg body weight/dose to about 50 mg/kg body weight/day, 10 mg/kg body weight/day to about 100 mg/kg body weight/day, and 10 mg/kg body weight/day to about 50 mg/kg body weight/day.

As used herein, “pharmaceutically acceptable carrier” includes any and all solvents, dispersion media, coatings, antibacterial and antifungal agents, isotonic and absorption delaying agents, sweeteners, salts, buffers, and the like. The pharmaceutically acceptable carriers may be prepared from a wide range of materials including, but not limited to, flavoring agents, sweetening agents and miscellaneous materials such as buffers and absorbents that may be needed in order to prepare a particular therapeutic composition.

In some cases, a composition comprising a delivery vehicle can be formulated under sterile conditions within a reasonable time prior to administration. For example, a composition comprising a delivery vehicle can be formulated from about 1 month, 2 weeks, 1 week, 5 days, 3 days, 2 days, 1 day, 10 hours, 5 hours, or immediately prior to administration to a subject. In an aspect, a delivery vehicle can be frozen and thawed prior to administration. Provided delivery vehicles can be used in combination with secondary therapies. For example, a secondary therapy such as chemotherapy or radiation therapy may be administered before or subsequent to the administration of a delivery vehicle, for example within 12 hr. to 7 days. A combination of therapies, such as both chemotherapy and radiation therapy may be employed in addition to the administration of the delivery vehicles

In some cases, provided delivery vehicles can comprise a coating. A coating can be an enteric coating. Enteric coatings can be utilized to prevent or minimize dissolution in the stomach but allow dissolution in the small intestine. In some embodiments, a coating can include an enteric coating. An enteric coating can be a barrier applied to oral medication that prevents release of medication before it reaches the small intestine. Delayed-release formulations, such as enteric coatings, can an irritant effect on the stomach from administration of a medicament from dissolving in the stomach. Such coatings are also used to protect acid-unstable drugs from the stomach's acidic exposure, delivering them instead to a basic pH environment (intestine's pH 5.5 and above) where they may not degrade.

Dissolution can occur in an organ. For example, dissolution can occur within a duodenum, jejunum, ilium, and/or colon, or any combination thereof. In some cases, dissolution can occur in proximity to a duodenum, jejunum, ilium, and/or colon. Some enteric coatings work by presenting a surface that is stable at a highly acidic pH found in the stomach, but break down rapidly at a less acidic (relatively more basic) pH. Therefore, an enteric coated pill may not dissolve in the acidic environment of the stomach but can dissolve in an alkaline environment present in a small intestine. Examples of enteric coating materials include, but are not limited to, methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), methyl methacrylate-methacrylic acid copolymers, sodium alginate and stearic acid.

An enteric coating can be applied at a functional concentration. An enteric coating can be cellulose acetate phthalate, Polyvinyl acetate phthalate, Hydroxypropylmethylcellulose acetate succinate, Poly(methacylic acid-co-ethyl acrylate) 1:1, Poly(methacrylic acid-co-ethyl acrylate) 1:1, Poly(methacylic acid-co-methyl methacrylate) 1:1, Poly(methacylic acid-co-methyl methacrylate) 1:1, Poly(methacylic acid-co-methyl methacrylate) 1:2, Poly(methacylic acid-co-methyl methacrylate) 1:2, Poly(methyl acrylate-co-methyl methacrylate-co-methacrylic acid) 7:3:1, or any combination thereof. An enteric coating can be applied from about 6 mg/(cm²) to about 12 mg/(cm²). An enteric coating can also be applied to a structure from about 1 mg/(cm²), 2 mg/(cm²), 3 mg/(cm²), 4 mg/(cm²), 5 mg/(cm²), 6 mg/(cm²), 7 mg/(cm²), 8 mg/(cm²), 9 mg/(cm²), 10 mg/(cm²), 11 mg/(cm²), 12 mg/(cm²), 13 mg/(cm²), 14 mg/(cm²), 15 mg/(cm²), 16 mg/(cm²), 17 mg/(cm²), 18 mg/(cm²), 19 mg/(cm²), to about 20 mg/(cm²).

In some embodiments, a pharmaceutical composition comprising a subject delivery vehicle can be orally administered from a variety of drug formulations designed to provide delayed-release. Delayed oral dosage forms include, for example, tablets, capsules, caplets, and may also comprise a plurality of granules, beads, powders or pellets that may or may not be encapsulated. Tablets and capsules can represent oral dosage forms, in which case solid pharmaceutical carriers can be employed. In a delayed-release formulation, one or more barrier coatings may be applied to pellets, tablets, or capsules to facilitate slow dissolution and concomitant release of drugs into the intestine. Typically, a barrier coating can contain one or more polymers encasing, surrounding, or forming a layer, or membrane around a therapeutic composition or active core. In some embodiments, active agents, such as a polynucleic acid, can be delivered in a formulation to provide delayed-release at a pre-determined time following administration. The delay may be up to about 10 minutes, about 20 minutes, about 30 minutes, about 1 hour, about 2 hours, about 3 hours, about 4 hours, about 5 hours, about 6 hours, or up to 1 week in length. In some cases, an enteric coating may not be used to coat a particle.

Polymers or coatings that can be used to achieve enteric release can be anionic polymethacrylates (copoly-merisate of methacrylic acid and either methyl-methacrylate or ethylacrylate (Eudragit®), cellulose based polymers, e.g. cellulose acetatephthalate (Aquateric®) or polyvinyl derivatives, e.g. polyvinyl acetate phthalate (Coateric®) in some cases.

In some cases, formulations can be presented in unit-dose or multi-dose containers, for example sealed ampoules and vials, and can be stored in a frozen or freeze-dried (lyophilized) condition requiring only the addition of sterile liquid carrier immediately prior to use. For oral administration, the compositions can take the form of, for example, tablets or capsules prepared by a conventional technique with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulphate). The tablets can be coated in some cases. Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration the compositions can take the form of tablets or lozenges formulated in conventional manner. In some cases, compositions can also be formulated as a preparation for implantation or injection. Thus, for example, a structure can be formulated with suitable polymeric, aqueous, and/or hydrophilic materials, or resins, or as sparingly soluble derivatives (e.g., as a sparingly soluble salt). The compounds can also be formulated in rectal compositions, creams or lotions, or transdermal patches.

In some cases, a pharmaceutical composition may include a salt. A salt can be relatively non-toxic. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed. It is intended that the following claims define the scope of the disclosure and that methods and structures within the scope of these claims and their equivalents be covered thereby.

### EXAMPLES

### Example 1: Preparation of Exemplary Delivery Vehicles of this Disclosure

This example provides an exemplary method of preparing a delivery vehicle of this disclosure. Lipid components of the delivery vehicle DODMA (Sigma Aldrich), Deoxycholate (Sigma Aldrich), MVL5 (Avanti Polar Lipids), DSPC (Avanti Polar Lipids), DMG-PEG 2000 (Avanti Polar Lipids), DOPC (Avanti Polar Lipids), DiI (ThermoFisher Scientific), DiO (ThermoFisher Scientific) were dissolved in ethanol and heated above their phase transition temperature, for instances where the phase transition temperatures were higher than 37° C. For example, when using DSPC, the lipids and aqueous phase were heated to 70° C. When using DOPC, the lipids and aqueous phase were not heated and used at room temperature. Nucleic acids were dissolved in an aqueous buffer heated above the phase transition temperature of the lipids.

The aqueous buffer pH was set at below the pKa of the bile salt and the cationic lipids. In this way, the lipids were strongly cationic when formulated with the nucleic acids. To form the delivery vehicle with cargo, the lipids and nucleic acids were mixed using microfluidic channels followed by removal of ethanol via dialysis. Other suitable methods can also be used for this step. For example, lipid structures such as liposomes can be formed by thin film hydration where the lipids may be dissolved in organic phase and dried using a rotovap under rotation. The thin film that is formed can be hydrated in water. The hydrated lipids can be heated to 70° C. for example, for DSPC or used at room temperature for example, for DOPC and extruded through the appropriate extruder pore size. The nucleic acid cargo can be mixed with the lipids to form lipoplexes.

Another suitable alternate method for the preparation of exemplary delivery vehicles is using the thin film hydration. Lipids are dissolved and mixed in an organic solvent. The solvent is removed, and the formed thin film is hydrated in an aqueous solution. The lipids are sized appropriately using sonication or extrusion. Nucleic acids can be complexed by mixing the lipid mixture and nucleic acids together.

### Formulation of the Exemplary Delivery Vehicles

To prepare exemplary delivery vehicles, containing encapsulated nucleic acid, 300 μg of plasmid DNA encoding for Gaussia luciferase under a cytomegalovirus (CMV) promoter was dissolved in a final volume of 3 mL 50 mM Sodium Acetate buffer (pH 4.8). Appropriate moles of MVL5, DODMA, Deoxycholate, MVL5, DSPC, DMG-PEG2000 and/or DOPC were mixed in ethanol according to their mole and cationic lipid: Nucleic acid ratio (See Table 2 for mole % of the lipids in the various formulations prepared). The cationic lipid: nucleotide molar ratio was maintained at about 16. Fluorescently labelled lipids, such as DiI and DiO, when used, were added to the mix at 0.5% of the total lipid moles. Ethanol volume was raised to 1 mL.

The nucleic acid is in the aqueous sodium acetate buffer phase in a 3 mL syringe. The lipids are in ethanol in a 1 mL syringe. The two syringes are mounted on to a NanoAssemblr (Precision Nanosystems) and then the two samples are mixed using the microfluidics chip on the NanoAssemblr.

For this study, samples were mounted into syringes (as mentioned above, nucleic acid in the 3 mL syringe and lipids in the 1 mL syringe) on a NanoAssemblr Benchtop and preheated to 65° C. for the DSPC formulations or at room temperature (about 25° C.) for the DOPC formulations. Samples were mixed using the NanoAssemblr Benchtop microfluidic chip system with a flow rate of 6 mL/min. pH was neutralized with 300 mM HEPES buffer at pH 7.5. Ethanol was removed using dialysis overnight. Samples were concentrated using Amicon Ultra-4 with a 100 kDa molecular weight cutoff.

TABLE 2
Example Formulations Prepared

| | Formulation | |
|---|---|---|
| Lipid Components | No. | Ratio of lipids (in mol %) |
| DODMA/DSPC/Deoxycholate/PEG-DMG | 1 | 40/31.6/25.9/2.5 |
| | 2 | 30/37.1/30.4/2.5 |
| | 3 | 20/42.6/34.9/2.5 |
| | 4 | 10/48.1/39.4/2.5 |
| | 5 | 25/37.1/30.4/7.5 |
| MVL5/DODMA/DSPC/Deoxycholate/DMG-PEG | 6 | 6.25/18.75/37.1/30.4/7.5 |
| | 7 | 12.5/12.5/37.1/30.4/7.5 |
| | 8 | 18.75/6.25/37.1/30.4/7.5 |
| MVL5/DSPC/Deoxycholate/DMG-PEG | 9 | 25/37.1/30.4/7.5 |
| DSPC/Deoxycholate | 10 | 55/45 |
| DOPC/Deoxycholate | 11 | 55/45 |
| DSPC/Deoxycholate/DMG-PEG | 12 | 53.6/43.9/2.5 |
| DSPC/Chol | 13 | 55/45 |
| MVL5/DODMA/DSPC/DMG-PEG | 14 | 17.3/17.3/58.1/7.1 |
| MVL5/DODMA/Deoxycholate/DMG-PEG | 15 | 19.5/19.5/52.8/8 |
| MVL5/DODMA/DSPC/Deoxycholate | 16 | 12.5/12.5/41.3/33.7 |

### Example 2: Transfection of Exemplary Delivery Vehicles of this Disclosure

In this study, the transfection efficiency of exemplary delivery vehicles (as prepared using the process described in Example 1 above) were assessed. HEK cells cultured to confluency between 50-80% were used for transfections. 1 μg of Gaussia luciferase expressing plasmid DNA encapsulated in a lipid nanoparticle (as listed above in Table 2) was used per well in a 24 well plate. Transfection efficiency was assessed by taking 30 μl of media after 24 h and performing a flash luciferase assay (Pierce Gaussia Luciferase Assay Kit). Increased value of relative light units (RLU) corresponded to greater transfection efficiency.

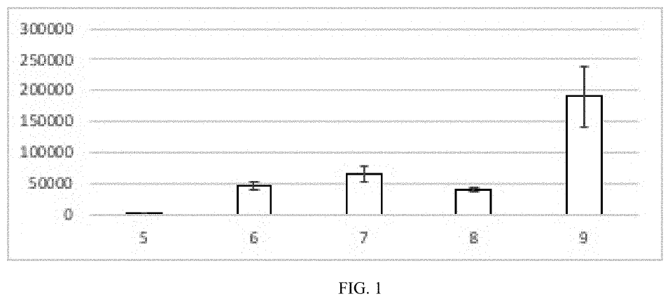

It was observed that the presence of the multivalent cationic lipid MVL5 increased the transfections significantly, possibly by exerting a positive or neutral character on the bile salt stable system. This was likely to due to increased endosomal escape. Due to its multivalency (+3 at physiological pH and +5 at lysosomal pH) and the high molar ratio of the negatively charged bile salts needed for stability, MVL5 and other multivalent lipids can be best suited for this system. Data is shown in FIG. 1.

### Example 3: Stability of Exemplary Delivery Vehicles of this Disclosure

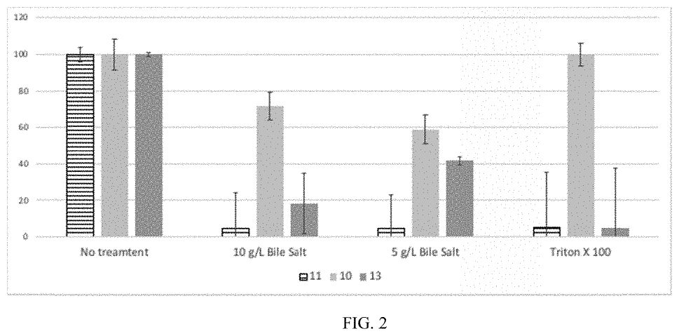

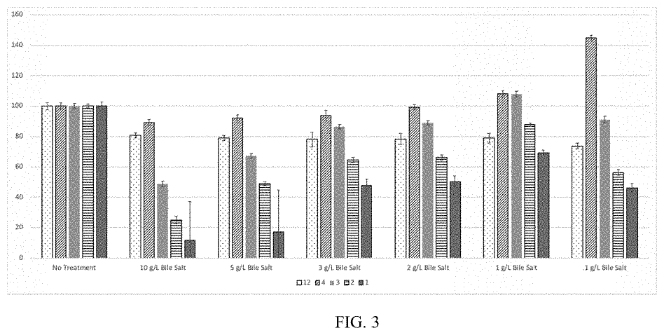

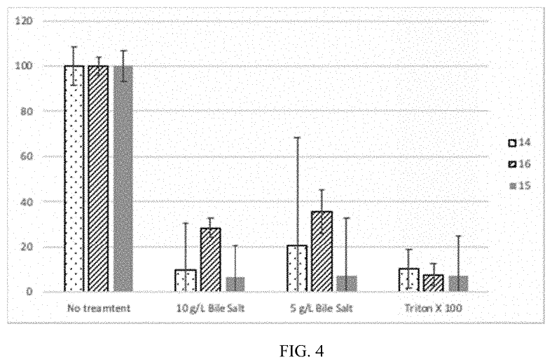

In this study, the stability of exemplary delivery vehicles, in a high bile salt environment, were assessed. To determine delivery vehicle stability, the delivery vehicles used in this assay incorporated 0.5 mol % each of DiI and DiO. DiI and DiO are fluorescent dyes that are FRET pairs. Bile salts were simulated by using an equal mixture of cholic acid and deoxycholate at indicated concentrations (in FIGS. 2-4). It was expected that if the delivery vehicle was susceptible to being disrupted by the bile salts, it would result in decreased FRET intensity. Relative fluorescence units (RFU) was determined by taking exciting at 465 nm and reading emission at 501 nm and 570 nm. The RFU reading at 570 nm was divided by the reading at 501 nm. The readings were normalized to the FRET intensity of the system without any treatment. Data is shown in FIG. 2, FIG. 3, and FIG. 4.

This study demonstrated that DSPC/Deoxycholate (as in Formulation No. 10) but not DOPC/Deoxycholate (as in Formulation No. 11) was stable to bile salts. It should be noted that DOPC/Deoxycholate is analogous to elastic liposomes were found to be highly susceptible to bile salts. In contrast, DSPC/Deoxycholate was found to be highly resistant to bile salt attack. Furthermore, DSPC/Cholesterol (as in Formulation No. 13), was also found to not be resistant to bile salts. This demonstrated that the presence of the saturated lipid tail was not enough to provide stability against bile salts and that the bile salts (e.g., Deoxycholate) must be incorporated within the lipid nanoparticle to provide the stability.

Furthermore, as seen in FIG. 4, it was observed that PEGylation (as in Formulation No. 16), was not necessary for stability but omitting a high phase transition temperature lipid (as in Formulation No. 15), or omission of a bile salt (as in Formulation No. 14), resulted in loss of bile salt stability of the delivery vehicle.

### Example 4: Encapsulation of Nucleic Acid in Exemplary Delivery Vehicles of this Disclosure

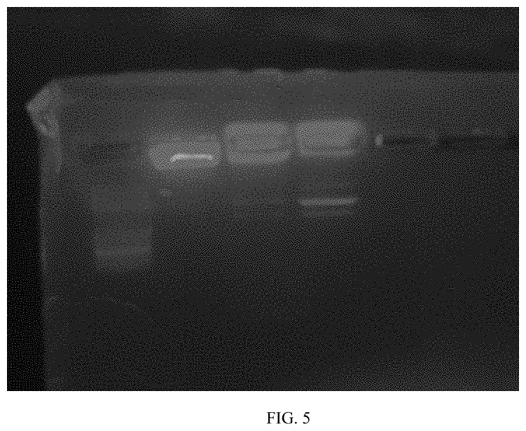

For this study, a delivery vehicle containing 1 μg of DNA encapsulated by the lipid nanoparticle (Formulation No. 5 in Table 2) was loaded to lanes of an agarose gel, either untreated (lane 2 in FIG. 5), (ii) treated with 7% Triton-X 100 (lane 3 in FIG. 5), (iii) treated with 7% Triton-X 100 plus 70° C. for 30 mins (lane 4 in FIG. 5), followed by electrophoresis. SYBR Safe was used to detect the DNA by UV light. No DNA band was found for any of the cationic lipid containing bile salt stable systems (lane 2, untreated), indicating encapsulation and that DNA was not released from the delivery vehicle; however, DNA bands were seen when the system was disrupted using detergent and heat (lanes 3 and 4), indicating that the vehicle was unstable in this environment and DNA was released upon treatment. Data is shown in FIG. 5. This demonstrated the benefit of having a cargo (such as DNA) encapsulated within a delivery vehicle that is stable in a bile salt environment, particularly for efficient protection in a high bile salt environment, such as the gastrointestinal tract.

### Example 5: Preparation of Delivery Vehicle with Cargo

Encapsulation of a nucleic acid cargo was performed as follows: lipids were dissolved in ethanol and heated above their phase transition temperature. The nucleic acid dissolved in an aqueous buffer heated above the phase transition temperature of the lipids. The aqueous buffer pH was set at below the pKa of the bile salt and the cationic lipids. In this way, the lipids were strongly cationic when formulated with the nucleic acids. The lipids and nucleic acids were mixed using microfluidic channels. The pH was raised to neutral and the sample was concentrated, and ethanol removed using dialysis.

Materials: DODMA (Sigma Aldrich), Deoxycholate (Sigma Aldrich), MVL5 (Avanti Polar Lipids), DSPC (Avanti Polar Lipids), DMG-PEG 2000 (Avanti Polar Lipids), DOPC (Avanti Polar Lipids), DiI (ThermoFisher Scientific), DiO (ThermoFisher Scientific), and GMO (MP Biomedicals)

### Formulation

375 ug of plasmid DNA encoding for Gaussia luciferase under a CMV promoter was dissolved in a final volume of 3 mL 50 mM Sodium Acetate buffer (pH 4.8). Appropriate moles of MVL5, DODMA, Deoxycholate, MVL5, DSPC, GMO, DMG-PEG2000 and/or DOPC were mixed in ethanol according to their mole and cationic lipid: Nucleic acid ratio. The cationic lipid: nucleotide molar ratio remained constant at 16. When lipids were fluorescently labelled with DiI and DiO, each DiI and DiO was added to the mix at 0.5% mol of the total lipid moles. Ethanol volume was raised to 1 mL. Samples were mounted into syringes on the Nanoassemblr Benchtop (Precision NanoSystems, CA) and preheated to 65° C. for the DSPC formulations or at room temperature for the DOPC formulations. Samples were mixed using the NanoAssemblr Benchtop microfluidic chip system with a flow rate of 6 mL/min. pH was neutralized and then ethanol was removed using dialysis overnight. Sample was concentrated using Amicon Ultra-4 with a 100 kDa cutoff (Merck Millipore Ltd, Ireland).

The following formulations were made as shown in Table 3.

TABLE 3
Example Formulations Prepared

| | Particle | |
|---|---|---|
| Formulation | # | Molar ratios |
| DODMA/DSPC/Deoxycholate | 1 | 25/41.25/33.75 |
| MVL5/DODMA/DSPC/Deoxycholate | 2 | 6.25/18.75/41.25/33.75 |
| MVL5/DODMA/DSPC/Deoxycholate | 3 | 12.5/12.5/41.25/33.75 |
| MVL5/DODMA/DSPC/Deoxycholate | 4 | 18.75/6.25/41.25/33.75 |
| MVL5/DODMA/DSPC/Deoxycholate/DMG-PEG | 5 | 12.4/12.4/40.8/33.4/1 |
| MVL5/DODMA/DSPC/Deoxycholate/DMG-PEG | 6 | 12.25/12.25/40.4/33.1/2 |
| MVL5/DODMA/DSPC/Deoxycholate/DMG-PEG | 7 | 12.1/12.1/40.0/32.7/3 |
| MVL5/DODMA/DSPC/Deoxycholate/DMG-PEG | 8 | 11.9/11.9/39.2/32.1/5 |
| MVL5/DODMA/DSPC/Deoxycholate/DMG-PEG | 9 | 11.25/11.25/37.1/30.4/10 |
| MVL5/DSPC/Deoxycholate | 10 | 25/41.25/33.75 |
| MVL5/DODMA/DOPC/Deoxycholate/DMG-PEG | 11 | 12.4/12.4/40.8/33.4/1 |
| MVL5/DODMA/GMO/Deoxycholate/DMG-PEG | 12 | 12.4/12.4/40.8/33.4/1 |
| MVL5/DODMA/DSPC/Deoxycholate/DSG-PEG | 13 | 12.4/12.4/40.8/33.4/1 |
| MVL5/DODMA/DSPC/Deoxycholate/DSG-PEG | 14 | 12.25/12.25/40.4/33.1/2 |
| MVL5/DODMA/DSPC/Deoxycholate/DSG-PEG | 15 | 12.1/12.1/40.0/32.7/3 |

In summary, particles with DMG-PEG were stable at even 1% DMG-PEG and did not form aggregates. DSG has a stearic acid lipid tail that is present in the gel phase at 37° C. DMG has a myristolic acid lipid tail that is in the liquid phase at 37° C. DMG-PEG was present in the liquid phase portion(s) of the vehicle and thus stabilized the cationic lipids preventing aggregation whereas DSG-PEG was in the gel phase portion(s) and could not provide the same stabilization effect.

### Example 6: In Vivo Administration of Delivery Vehicles

Mice were dosed intrarectally with approximately 30 micrograms of DNA encapsulated in nanoparticles that were DiI and DiO labelled. 4 hours after dosing, mice were sacrificed, and the intestines were embedded in OCT and frozen in dry ice and stored at −80° C. The tissues were cryosectioned into 30 micrometer slices and imaged using a BioTek Cytation 1. DiI fluorescence was measured in the RFP channel.

### PEGylated Particles Fail to Reach the Intestinal Epithelial Cells

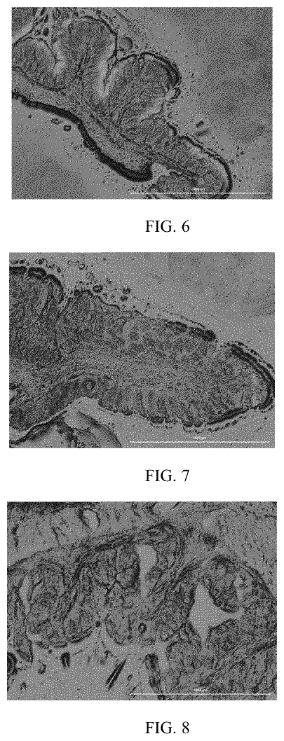

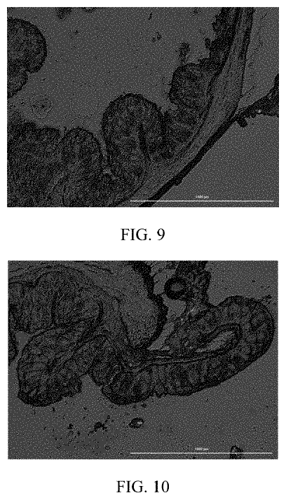

MVL5/DODMA/DSPC/Deoxycholate/DMG-PEG (Particles 5-9) particles were formed with increasing amounts of DMG-PEG and the behavior of the particles was investigated in vivo. An increasing amount of DMG-PEG resulted in decreased distribution at the intestinal tissue. This is in contradiction with the current dogma of increasing PEGylation to increase intestinal epithelial reach. We believe that increased PEGylation reduces the exposure of the positive charge at the surface through its shielding properties. This reduces the dual nature of the particle, as shown in FIG. 6 (Particle 5), FIG. 7 (Particle 6), FIG. 8 (Particle 7), FIG. 9 (Particle 8), and FIG. 10 (Particle 9).

### Example 7: Delivery Vehicle In Vivo Testing

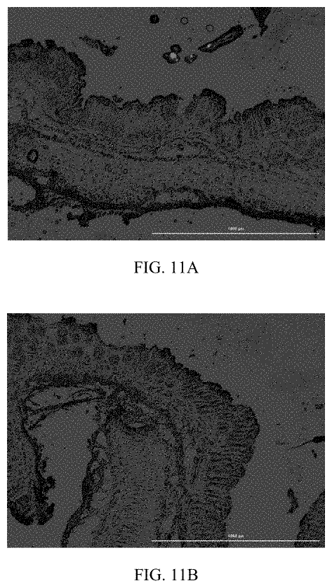

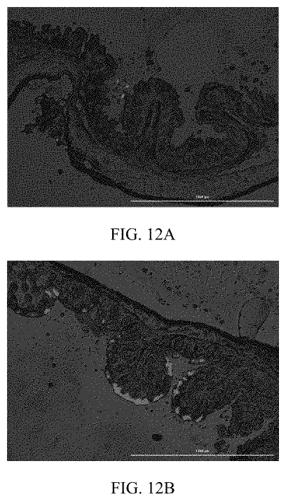

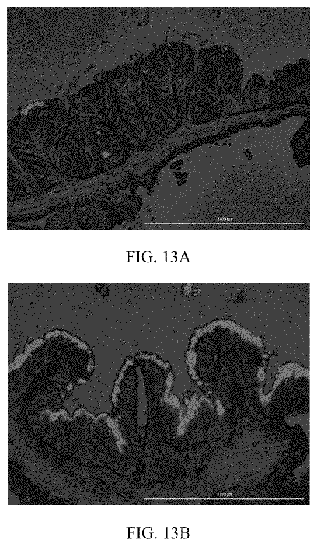

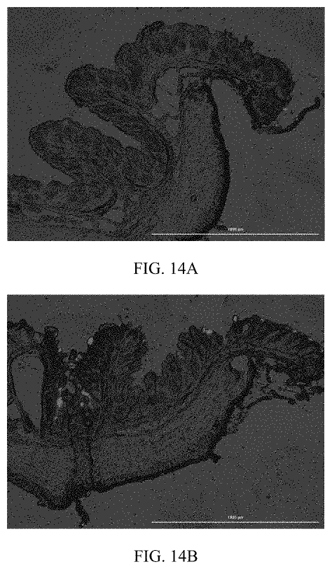

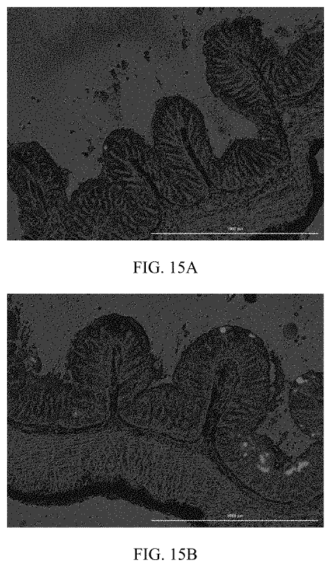

As shown in FIG. 11A, FIG. 11B, FIG. 12A, FIG. 12B, FIG. 13A, FIG. 13B, FIG. 14A, and FIG. 14B, the ratios of MVL5/DODMA were altered in DSPC/Deoxycholate/DMG-PEG) with DiI and DiO to investigate the effect of increasing positive charge. The following ratios of MVL5/DODMA in the particles were formed (0%/25%), (6.25%/18.75%), (12.5%/12.5%), (18.75%, 6.25%), (25%/0%). As DODMA is mostly neutral at neutral pH and is monovalent, the negative charge of deoxycholate and the multivalent charges of MVL5 dominated the behavior of the particle. Increasing MVL5, thereby increases charge.

Data shows the 12.5%/12.5% MVL5/DODMA ratio to be optimal for intestinal epithelia distribution of the particles in vivo. Too much MVL5 provided too strong of a cationic character resulting in adhesion to the negatively charged mucus. Too low of MVL5 resulted in negatively charged particles that may have repelled the mucus or had no interaction. Further, MVL5/DODMA/DSPC/Chol/DMG-PEG particles were made, and they were found to not reach the intestinal epithelial cells. In summary, dual charges are needed to reach the intestinal epithelial cells in a careful balance of charge, as shown in FIG. 11A, FIG. 11B, FIG. 12A, FIG. 12B, FIG. 13A, FIG. 13B, FIG. 14A, and FIG. 14B.

### Example 8: Zwitterionic Delivery Vehicles Vs. Dual Phase Delivery Vehicles

Delivery vehicles were generating as described in example 1 and were tested in vivo as described in Example 7. Zwitterionicity has been previously shown to increase mucus penetration without the presence of PEG. To investigate if zwitterionicity but not the dual phase nature is sufficient, particles were formulated where the particles were designed to be of a single phase. In order to make single phase particles, low phase transition temperature lipids (i.e. containing DOPC or GMO) were substituted instead of DSPC. The charges were held the same across the particles. Particles that were only liquid phase (containing DOPC or GMO instead of DSPC) were found to have significantly reduced or very little intestinal epithelial cell reach.

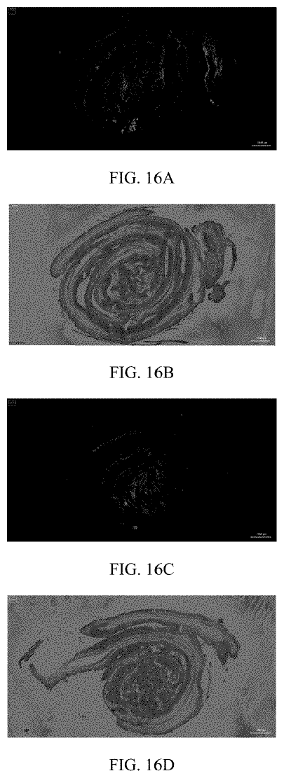

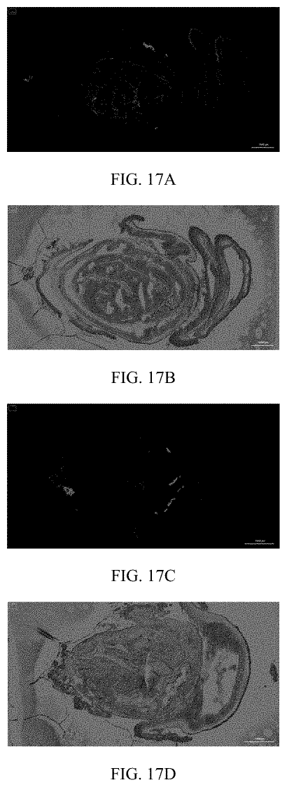

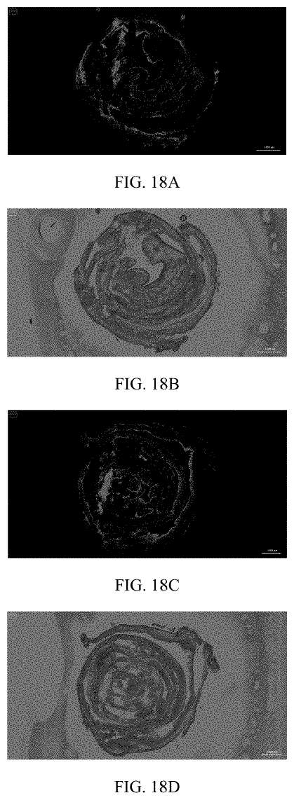

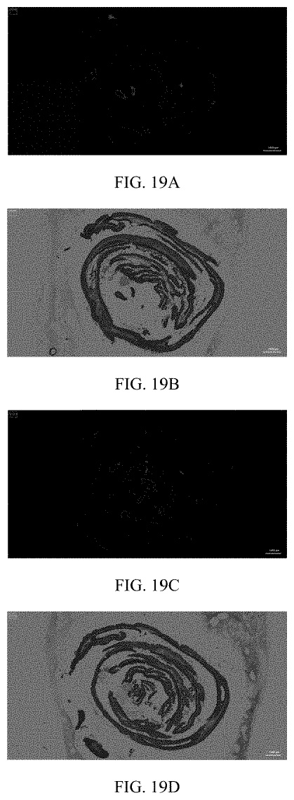

In summary, data shows that the presence of zwitterionicity alone is insufficient to allow for intestinal epithelial cell reach, as shown in FIG. 16A, FIG. 16B, FIG. 16C, and FIG. 16D.

### Example 9: Stability of Delivery Vehicles with Bile Salts

The following formulations were made using methods previously described in Example 1: MVL5:MC2 (Biofine International LLC, Vancouver BC Canada): Bile Salt:DSPC:DMG-PEG2000:DiI:DiO in the mol ratio of 0.96:0.96:2.592:3.168:0.0768:0.0384:0.0384 where the bile salt component was either ursodiol, deoxycholate, lithocholate, isolithocholate, alloisolithocholate, dehydrolithocholate or 5beta-cholanic acid. No nucleic acid was incorporated into the lipid nanoparticles. Alternate formulations can also be generated such as those provided in Table 4.

TABLE 4
Suitable alternate bile salt formulations

| Formulation | Molar Ratio |
|---|---|
| MVL5/MC2/DSPC/Deoxycholate/DMPE-PEG | 2.4/2.4/7.9/6.48/0.192 |
| MVL5/CL1H6/DSPC/Deoxycholate/DMG-PEG | 2.4/2.4/7.9/6.48/0.192 |
| MVL5/CL4H6/DSPC/Deoxycholate/DMG-PEG | 2.4/2.4/7.9/6.48/0.192 |
| MVL5/MC2/DSPC/Chenodeoxycholate/DMG-PEG | 2.4/2.4/7.9/6.48/0.192 |
| MVL5/MC2/DMPC/Deoxycholate/DMG-PEG | 2.4/2.4/7.9/6.48/0.192 |
| MVL5/MC2/DMPC/Deoxycholate/DMPE-PEG | 2.4/2.4/7.9/6.48/0.192 |
| MVL5/CL1H6/DMPC/Deoxycholate/DMG-PEG | 2.4/2.4/7.9/6.48/0.192 |
| MVL5/MC2/DSPC/Deoxycholate/Lithocholate/DMG-PEG | 2.4/2.4/7.9/5.2/1.3/0.192 |
| MVL5/CL1H6/DSPC/Deoxycholate/Lithocholate/DMG-PEG | 2.4/2.4/7.9/5.2/1.3/0.192 |
| MVL5/MC2/DSPC/Alloisolithocholate/DMG-PEG | 2.4/2.4/7.92/6.48/0.192 |
| MVL5/MC2/DSPC/Dehydrolithocholate/DMG-PEG | 2.4/2.4/7.92/6.48/0.192 |

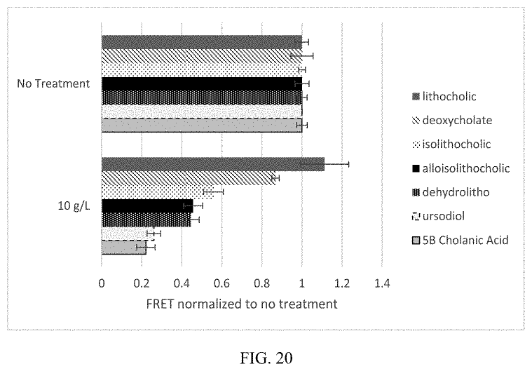

Stability of the lipid nanoparticles in bile salt was measured as discussed previously up to 10 g/L. FRET signal from DiI and DiO were normalized to no treatment. Levels of stability of the vehicles, in salt form, is shown in FIG. 20.

1-100. (canceled)

101. A delivery vehicle comprising a lipid composition, wherein said lipid composition comprises,
at least one saturated lipid,
at least one unsaturated cationic lipid or at least one unsaturated non-cationic lipid, and
at least one bile salt.

102. The delivery vehicle of claim 101, wherein the at least one unsaturated cationic lipid comprises at least two (2) unsaturated cationic lipids.

103. The delivery vehicle of claim 102, wherein the lipid composition comprises a conjugated lipid.

104. The delivery vehicle of claim 102, wherein the at least two unsaturated cationic lipids comprises at least one of: Dimethyldioctadecylammonium, 1,2-dialkyl-sn-glycero-3-ethylphosphocholine, 1,2-dialkyl-3-dimethylammonium-propane, 1,2-dialkyl-3-trimethylammonium-propane, 1,2-di-O-alkyl-3-trimethylammonium propane, 1,2-dialkyloxy-3-dimethylaminopropane, N,N-dialkyl-N,N-dimethylammonium, N-(4-carboxybenzyl)-N,N-dimethyl-2,3-bis(alkyloxy)propan-1-aminium, 1,2-dialkyl-sn-glycero-3-[(N-(5-amino-1-carboxypentyl)iminodiacetic acid)succinyl], N1-[2-((1S)-1-[(3-aminopropyl)amino]-4-[di(3-amino-propyl)amino]butylcarboxamido)ethyl]-3,4-di[alkyl]-benzamide, 1,2-Dialkyloxy-N,N-dimethylaminopropane, 4-(2,2-diocta-9,12-dienyl-[1,3]dioxolan-4-ylmethyl)-dimethylamine, O-alkyl ethylphosphocholines, 1,2-Dioleyloxy-3-(dimethylamino)propane (DODMA), 6Z,9Z,28Z,31Z)-heptatriaconta-6,9,28,31-tetraen-19-yl 3-(dimethylamino)propanoate (MC2), 3ß-[N—(N′,N′-dimethylaminoethane)-carbamoyl]cholesterol, N4-Cholesteryl-Spermine, 7-(4-(dimethylamino)butyl)-7-hydroxytridecane-1,13-diyl dioleate (CL1H6), N 1-[2-((1 S)-1-[(3-aminopropyl)amino]-4-[di(3 amino-propyl)amino]butylcarboxamido)ethyl]-3,4-di[oleyloxy]-benzamide (MVL5) and any combinations thereof.

105. The delivery vehicle of claim 101, wherein the at least one saturated lipid comprises a saturated non-cationic lipid.

106. The delivery vehicle of claim 105, wherein the concentration of the at least one unsaturated cationic lipid or unsaturated non-cationic lipid in the lipid composition is less than 50 mole % of the total lipid concentration of the lipid composition, and wherein the saturated non-cationic lipid that has a phase transition temperature of at least about 37° C.

107. The delivery vehicle according to claim 101, wherein the delivery vehicle is stable in a high bile salt environment, compared to an otherwise identical delivery vehicle that does not comprise the bile salt, optionally wherein the high bile salt environment comprises the environment in the gastrointestinal environment.

108. The delivery vehicle of claim 107, wherein the delivery vehicle demonstrates an increased stability in a solution containing at least about 5 g/L of the bile salt, compared to an otherwise identical delivery vehicle that does not comprise the bile salt, wherein the stability is measured by relative fluorescence intensity of a fluorescent lipid incorporated into the lipid nanoparticle, in a Forster resonance energy transfer (FRET) assay.

109. The delivery vehicle of claim 107, wherein the high bile salt environment comprises the environment in the gastrointestinal environment.

110. The delivery vehicle of claim 101, wherein the at least one bile acid comprises at least one of cholic acid, cholate, deoxycholic acid, deoxycholate, hyodeoxycholic acid, hyodeoxycholate, glycocholic acid, glycocholate, taurocholic acid, taurocholate, chenodeoxycholic acid, chenodeoxycholate, lithocholic acid, lithocolate, and the like, or salts thereof. In some embodiments, a bile acid is ursodiol, isolithocholate, alloisolithocholate, dehydrolithochlate, 5-beta-cholanic acid, and any combination thereof.

111. A delivery vehicle comprising a lipid nanoparticle, wherein the lipid nanoparticle comprises,
at least one multivalent cationic lipid,
at least one unsaturated cationic lipid,
at least one structural lipid, and
at least one bile salt.

112. The delivery vehicle of claim 111, wherein the at least one multivalent cationic lipid comprises at least one of MVL5, N4-Cholesteryl-Spermine HCl (GL67), and any combinations thereof.

113. The delivery vehicle of claim 112, wherein the at least one unsaturated cationic lipid comprises at least one of: Dimethyldioctadecylammonium, 1,2-dialkyl-sn-glycero-3-ethylphosphocholine, 1,2-dialkyl-3-dimethylammonium-propane, 1,2-dialkyl-3-trimethylammonium-propane, 1,2-di-O-alkyl-3-trimethylammonium propane, 1,2-dialkyloxy-3-dimethylaminopropane, N,N-dialkyl-N,N-dimethylammonium, N-(4-carboxybenzyl)-N,N-dimethyl-2,3-bis(alkyloxy)propan-1-aminium, 1,2-dialkyl-sn-glycero-3-[(N-(5-amino-1-carboxypentyl)iminodiacetic acid)succinyl], N1-[2-((1S)-1-[(3-aminopropyl)amino]-4-[di(3-amino-propyl)amino]butylcarboxamido)ethyl]-3,4-di[alkyl]-benzamide, 1,2-Dialkyloxy-N,N-dimethylaminopropane, 4-(2,2-diocta-9,12-dienyl-[1,3]dioxolan-4-ylmethyl)-dimethylamine, O-alkyl ethylphosphocholines, DODMA, MC2, 3ß-[N—(N′,N′-dimethylaminoethane)-carbamoyl]cholesterol, CL1H6, and any combinations thereof.

114. The delivery vehicle of claim 113, wherein the concentration of the at least one unsaturated cationic lipid is about 50 mole % or less of the total lipid concentration.

115. The delivery vehicle of claim 112, wherein the concentration of the at least one multivalent cationic lipid is about 50 mole % or less of the total lipid concentration.

116. The delivery vehicle of claim 111, wherein the delivery vehicle comprises least one of distearoylphosphatidylcholine (DSPC), phosphatidylcholine 1,2-dimyristoyl-sn-glycero-3-phosphocholine (DMPC), 1,2-Distearoyl-sn-glycero-3-phospho-L-serine (DSPS), dioleoylphosphatidylcholine (DOPC), dipalmitoylphosphatidylcholine (OPEC), dioleoylphospbatidylglycerol (DOPG), dipahnitoylphosphatidylglycerol (DPPG), dioleoyl-phosphatidylethanolamine (DOPE), palmitoyloleoylphosphatidylcholine (POPC), palmitoylolmyl-phosphatidylethanolamine (POPE) and dioleoyl-phosphatidylethanolamine 4-(4-maleimidomethyl)cyelohexane-1-carboxylate (DOPE-teal), dipahnitoyl phosphatidyl ethanolamine (DPPE), dimyristoylphosphoetbanolamine (DMPE), distearoylphosphatidylethanolamine (DSPE), 16-O-monomethyl PE, 16-Odimethyl PE, 18-1-trans PE, 1-stearoyl-2-oleoyl-phosphatidyethanolamine (SOPS), 1,2-dielaidoyl-sn-glycero-3-phophoethanolamine (transDOPE), and any combinations thereof.

117. The delivery vehicle of claim 116, wherein the at least one bile acid comprises at least one of cholic acid, cholate, deoxycholic acid, deoxycholate, hyodeoxycholic acid, hyodeoxycholate, glycocholic acid, glycocholate, taurocholic acid, taurocholate, chenodeoxycholic acid, chenodeoxycholate, lithocholic acid, lithocolate, and the like, or salts thereof. In some embodiments, a bile acid is ursodiol, isolithocholate, alloisolithocholate, dehydrolithochlate, 5-beta-cholanic acid, and any combination thereof.

118. The delivery vehicle of claim 117, wherein the concentration of the at least one bile salt is between about 80 mole % to about 10 mole %.

119. The delivery vehicle of claim 111, comprising at least one conjugated lipid.

120. The delivery vehicle according to claim 111, wherein the delivery vehicle is stable in a high bile salt environment, compared to an otherwise identical delivery vehicle that does not comprise the bile salt wherein the stability is measured by relative fluorescence intensity of a fluorescent lipid incorporated into the lipid nanoparticle, in a FRET assay.


SEQUENCE LISTING (16 sequences)

SEQ ID NO: 1 - protein, 7 aa, Artificial Sequence
  Description of Artificial SequenceSynthetic Amino Acid Sequence
PKKKRKV

SEQ ID NO: 2 - protein, 16 aa, Artificial Sequence
  Description of Artificial SequenceSynthetic Amino Acid Sequence
KRPAATKKAGQAKKKK

SEQ ID NO: 3 - protein, 9 aa, Artificial Sequence
  Description of Artificial SequenceSynthetic Amino Acid Sequence
PAAKRVKLD

SEQ ID NO: 4 - protein, 11 aa, Artificial Sequence
  Description of Artificial SequenceSynthetic Amino Acid Sequence
RQRRNELKRSP

SEQ ID NO: 5 - protein, 38 aa, Artificial Sequence
  Description of Artificial SequenceSynthetic Amino Acid Sequence
NQSSNFGPMKGGNFGGRSSGPYGGGGQYFAKPRNQGGY

SEQ ID NO: 6 - protein, 42 aa, Artificial Sequence
  Description of Artificial SequenceSynthetic Amino Acid Sequence
RMRIZFKNKGKDTAELRRRRVEVSVELRKAKKDEQILKRRNV

SEQ ID NO: 7 - protein, 8 aa, Artificial Sequence
  Description of Artificial SequenceSynthetic Amino Acid Sequence
VSRKRPRP

SEQ ID NO: 8 - protein, 8 aa, Artificial Sequence
  Description of Artificial SequenceSynthetic Amino Acid Sequence
PPKKARED

SEQ ID NO: 9 - protein, 8 aa, Artificial Sequence
  Description of Artificial SequenceSynthetic Amino Acid Sequence
PQPKKKPL

SEQ ID NO: 10 - protein, 12 aa, Artificial Sequence
  Description of Artificial SequenceSynthetic Amino Acid Sequence
SALIKKKKKMAP

SEQ ID NO: 11 - protein, 5 aa, Artificial Sequence
  Description of Artificial SequenceSynthetic Amino Acid Sequence
DRLRR

SEQ ID NO: 12 - protein, 10 aa, Artificial Sequence
  Description of Artificial SequenceSynthetic Amino Acid Sequence
RKLKKKIKKL

SEQ ID NO: 13 - protein, 10 aa, Artificial Sequence
  Description of Artificial SequenceSynthetic Amino Acid Sequence
REKKKFLKRR

SEQ ID NO: 14 - protein, 20 aa, Artificial Sequence
  Description of Artificial SequenceSynthetic Amino Acid Sequence
KRKGDEVDGVDEVAKKKSKK

SEQ ID NO: 15 - protein, 17 aa, Artificial Sequence
  Description of Artificial SequenceSynthetic Amino Acid Sequence
RKCLQAGMNLEARKTKK

SEQ ID NO: 16 - protein, 7 aa, Artificial Sequence
  Description of Artificial SequenceSynthetic Amino Acid Sequence
PKQKKRK